(19) United States
(12) Patent Application Publication
(10) Pub. No.: US 2005/0043262 A1
(43) Pub. Date: Feb. 24, 2005

(54) Novel specific inhibitor of the cyclin kinase inhibitor p21Waf1/Cip1 and methods of using the inhibitor

(76) Inventor: Robert H. Weiss, Vacaville, CA (US)

Correspondence Address: MANDEL & ADRIANO, 55 SOUTH LAKE AVENUE, SUITE 710, PASADENA, CA, 91101, US

(21) Appl. No.: 10/867,951

(22) Filed: Jun. 14, 2004

Related U.S. Application Data
(63) Continuation-in-part of application No. 10/240,140, filed on Sep. 26, 2002.
(60) Provisional application No. 60/193,155, filed on Mar. 29, 2000.

(51) Int. Cl. A61K 48/00; A61K 39/395

(52) U.S. Cl. 514/44; 424/146.1

(57) ABSTRACT

The present invention provides methods and compositions for regulating abnormal cell growth and proliferation mediated by p21^{Waf1/Cip1} using inhibitors of p21^{Waf1/Cip1}.

This application is a continuation-in-part of U.S. Ser. No. 10/240,140, filed Sep. 26, 2002, which corresponds to PCT Application No. PCT/US01/10443, filed Mar. 29, 2001, which claims the priority of U.S. Ser. No. 60/193,155, filed Mar. 29, 2000, the contents of which are hereby incorporated by reference in their entirety.

Throughout this application various publications are referenced. The disclosures of these publications, in their entireties, are hereby incorporated by reference into this application, in order to more fully describe the state of the art to which this invention pertains.

### FIELD OF INVENTION

The present invention relates to methods and compositions for regulating cell growth and proliferation, mediated by cyclin-dependent kinases, by inhibiting p21^{Waf1/Cip1}, and more particularly to the prevention and treatment of diseases associated with abnormal proliferation of cells, using p21^{Waf1/Cip}1 inhibitory agents.

### BACKGROUND OF THE INVENTION

The mechanism by which cells “decide” whether to grow or not to grow is of paramount importance in a surprising variety of diseases. Unregulated or abnormal cell growth in vascular smooth muscle (VSM) and phenotypically-related glomerular mesangial cells (Dubey R K, et al. Curr Opin Nephrol Hypertens 6:88-105, 1997), is the underlying pathogenic mechanism in such diverse diseases as hemodialysis graft stenosis, angioplasty restenosis (Ross, 1993 Nature, 362, p. 801-809), and atherosclerosis, as well as in mesangial proliferative kidney disease (Megyesi et al., 1999 Proc. Natl. Acad. Sci. US.A., 96, p. 10830-10835). It has been suggested that pharmacological methods to increase p21^{Waf1/Cip1} may be useful in preventing the VSM cell proliferation seen after coronary angioplasty (Kusama et al., 1999 Atherosclerosis, 143, p. 307-313; Takahashi et al., 1999 Circ. Res., 84, p. 543-550; and Yang et al., 1996: Semin. Interv. Cardiol., 1, p. 181-184). Thus, research directed at understanding the mechanisms by which these processes occur in VSM cells is critical to the development of specific therapies for these diseases.

Most likely due to the fact that protection from these often deadly diseases has provided significant survival advantage over evolutionary time, organisms have evolved complex and often redundant systems for keeping these essential, but potentially lethal, cellular processes, such as unregulated cell growth, in check. Consequently, there exist cell cycle regulators at multiple levels of the cell growth hierarchy: from the growth factor receptor regulatory proteins (such as receptor phosphorylation events and various G-proteins), through the cytoplasmic signal protein interactions (such as the mitogen-activated protein serine/threonine kinase (MAPK), stress-activated protein kinase (SAPK), and Janus family of protein kinase-signal transducers and activators of transcription (JAK-STAT) systems (Frye, R. A. in Oncogenes and Tumor Suppressor Genes in Human Malignancies, Benz C C and Liu, E T, eds. 63:281-299, Kluwer Academic Publishers, Boston), to the nuclear transcriptional control machinery (such as the cyclins, cyclin kinases, and cyclin kinase inhibitors) (Lavoie J N, et al. Prog Cell Cycle Res 2:49-58, 1996).

Mechanisms and regulation of the cyclin kinase system in VSM cells have been studied. These molecules (such as the cyclins, cyclin kinases, and cyclin kinase inhibitors), which regulate cell growth, are very distal along the growth factor signaling pathways, and may, therefore, be among the ultimate arbiters of the decision a cell must make whether it will proceed through the cell cycle and lead to the production of cellular progeny or die. Since many growth-controlling signaling pathways converge on the cyclin system, elucidating the mechanism of regulation of these molecules will likely lead to the development of pharmaceuticals which target these molecules and, consequently, are useful for the treatment of vascular and renal diseases as well as cancer.

Cell cycle progression is finely regulated by the interplay between the cyclin-dependent kinases (cdks) and the cdk inhibitors (CKIs). Cyclin is a protein involved in the cell cycle that accumulates during interphase and is destroyed during mitosis. Cdks are a well-conserved family of serine/threonine protein kinases, found in yeast and in at least eight different animal cells, which function in mitogenic signaling through their activation by the cyclins. This in turn leads to a cascade of events whereby the mitogen-stimulated cyclin D-dependent kinases phosphorylate retinoblastoma protein (Rb), causing release of inhibition of the transcription factor family known as E2F, and allowing S-phase specific gene transcription and subsequent progression through the G1/S transition (Sherr and Roberts, 1999, Genes and Dev., 13, p. 1501-1512).

The Cip/Kip family of CKIs (p21^{Waf1/Cip1}, p27^{Kip1}, and p57^{Kip2}) regulate the activity of the cyclin/cdk complex and have been shown to negatively regulate the process of cyclin-mediated cell cycle progression through inhibition of the cdks (p21^{Waf1/Cip1} (Gu et al. 1993, Nature, 366, 707-710; Harper et al., 1993, Cell, 75, 387-400; El-Deiry et al. 1993, Cell, 75, 817-825; Xiong et al. 1993, Nature, 366, 701-704; Dulic et al. 1994, Cell, 76, 1013-1023; Noda et al. 1994, Exp. Cell Res., 211, 90-98), p27^{Kip1} (Polyak et al. 1994, Genes & Dev. 8, 9-22; Polyak et al. 1994, Cell, 78, 59-66; and Toyoshima and Hunter, 1994, Cell, 78, 67-74), and p57^{Kip2} (Lee et al. 1995, Genes & Dev. 9, 639-649; and Matsuoka et al. 1995, Genes & Dev. 9, 650-662)).

The protein p21^{Waf1/Cip1} was first described in 1992 (Xiong et al., 1992 Cell, 71, p. 505-514). The sequences of the human, rat and mouse p21^{Waf1/Cip}1 genes are known (GenBank entries CAB06656, I84725 and I49023, respectively), and polyclonal and monoclonal antibodies, to human and rodent species, are commercially available. The human protein has been expressed in E. coli by commercial sources (Santa Cruz Biotechnology, Santa Cruz, Calif.).

The net result of induction or overexpression of the CKIs (particularly those in the Cip/Kip family) generally is cell cycle inhibition and growth suppression in VSM and other cell types (Chang et al., 1995 J. Clin. Invest., 96, p. 2260-2268; Ishida et al., 1997 J. Biol. Chem., 272, p. 10050-10057; Matsushita et al., 1998 Hypertension, 31, p. 493-498; Sewing et al., 1997 Mol. Cell Biol., 17, p. 5588-5597; and Weiss et al., 1999 J. Am. Soc. Nephrol., 9, p. 1880-1890). Consistent with this, the CKIs are down-regulated in response to a variety of mitogens, and overexpression of these molecules leads to growth arrest (Kato et al., 1994 Cell, 79, p. 487-496; Nourse et al., 1994 Nature, 372, p. 570-573; Pagano et al., 1995 Science, 269, p. 682-685; and Resnitzky et al., 1995 Mol. Cell Biol., 15, p. 4347-4352).

While much of the early work on the CKIs has focused on their role as growth inhibitors, it had been somewhat puzzling that expression of these molecules increases early after mitogen stimulation (Depoortere et al., 1996 J. Cell Sci., 109 (Pt 7), p. 1759-1764; and Michieli et al., 1994 Cancer Res., 54, p. 3391-3395). CKIs have also been implicated in positive effects on cyclin/cdk activation (Cheng et al., 1998 Proc. Natl. Acad. Sci. U.S.A., 95, p. 1091-1096; LaBaer et al., 1997 Genes Dev., 11, p. 847-862; and Zhang et al., 1994 Genes Dev., 8, p. 1750-1758). This led to more recent data showing the ability of some CKIs to take part in formation of the cyclin/cdk complexes, and thus to serve as “assembly factors” important for promoting cyclin/cdk association (Hiyama et al., 1998 Oncogene, 16, p. 1513-1523; and LaBaer et al., 1997 Genes Dev., 11, p. 847-862). Various CKIs have also been reported to act as “assembly factors” in other cells, both in vivo (Cheng et al., 1999 EMBO J., 18, p. 1571-1583) and in vitro (LaBaer et al., 1997: Genes Dev., 11, p. 847-862). In support of this role for the CKIs, others have shown that assembly of cyclin D1/D2-cdk4 complexes was impaired in fibroblasts from mice lacking the p21^{Waf1/Cip1} and/or p27^{Kip1} genes (Cheng et al., 1999 EMBO J., 18, p. 1571-1583), and that both p21^{Waf1/Cip}1 and p27^{Kip1} actively promoted interaction between the cyclin Ds and their counterpart cdks by stabilizing this complex (LaBaer et al., 1997 Genes Dev., 11, p. 847-862). However, primary fibroblasts from p21- and p27-null mice did not show overtly abnormal cell cycles, despite the finding by those investigators that overall cyclin D-dependent kinase activity was reduced below the assay limit of detectability. Previous studies have not shown inhibition of growth with interference of cyclin/cdk association. The cyclin D1/cdk 4 interaction occurs early after growth factor stimulation (reviewed in Arellano and Moreno, 1997 Int. J. Biochem. Cell Biol., 29, p. 559-573) and this interaction is facilitated by p21^{Waf1/Cip1} and p27^{Kip1} in vivo (LaBaer et al., 1997: Genes Dev., 11, p. 847-862).

Thus, CKIs exhibit both positive and negative effects on growth and apoptosis in a variety of cell types, including VSM cells. A further example of this is the mechanism of action of the HMG CoA reductase inhibitors, where accelerated graft atherosclerosis in heart, and probably renal, transplant patients is attenuated by the statins (Katznelson S, et al. Transplantation 61:1469-1474, 1996; and Southworth M R, Mauro V F. Ann Pharmacol 31:489-491, 1997). Many of the statins have been shown to attenuate smooth muscle growth and promote apoptosis in association with an increase in the cyclin kinase inhibitors p21 and p27 (Baetta R, et al. Pharmacol Res 36:115-121, 1997; Terada Y, et al. J. Am. Soc Nephrol 9:2235-2243, 1999; Weiss R H, et al. J Am Soc Nephrol 9:1880-1890, 1999; and Laufs U, et al. J Biol Chem 274:21926-21931, 1999), although whether this is the mechanism of this effect is unknown. Tumor cells that are p21(−/−) are also known to be sensitized to apoptosis (Stewart Z A, et al. Cancer Res 59:3831-3837, 1999; Fan S et al. Oncogene 14:2127-2136, 1997). What constitutes the “switch” from positive to negative effects of the cyclin kinase inhibitors on both growth and apoptosis is unclear.

There is a need for improved therapies of diseases, associated with abnormal cell growth and proliferation, that take into account the various pathways that result in stimulation of growth and of cells. For the first time, it is shown in the present invention that p21^{Waf1/Cip}1 serves a permissive role in platelet-derived growth factor (PDGF)-mediated VSM cell proliferation, such that its presence is required for the mitogenic effect of PDGF. It is thus possible to devise therapeutic strategies to inhibit cell proliferation, in proliferative diseases, by controlling the expression of CKIs, in particular p21^{Waf1/Cip1}.

### SUMMARY OF THE INVENTION

Accordingly, the present invention provides novel methods and compositions for regulating cell growth and proliferation, and treating diseases associated with abnormal cell growth and proliferation, mediated by cyclin-dependent kinases, by inhibiting p21^{Waf1/Cip}1 in cells expressing p21^{Waf1/Cip1}, using p21^{Waf1/Cip1} inhibitory agents. The methods are also for preventing and treating fibrotic diseases associated with abnormal cell growth and proliferation. The methods, further include, inhibiting angiogenesis and tumor growth by inhibiting p21^{Waf1/Cip}1 in cells expressing p21^{Waf1/Cip1}, using p21^{Waf1/cip}1 inhibitory agents. The methods include using inhibitory agents such as an antisense oligonucleotide of p21^{Waf1/Cip1} and anti-p21^{Waf1/Cip}1 antibodies, to inhibit transcription and/or expression of p21^{Waf1/Cip1}.

The therapeutic methods of the invention can also be used in conjunction with radiation therapy and chemotherapy.

### BRIEF DESCRIPTION OF THE FIGURES

FIG. 1A-D are bar graphs showing that antisense p21^{Waf1/Cip1} oligodeoxynucleotide transfection inhibits VSM cell DNA synthesis in a dose-dependent manner, as described in Example I, infra. A10 VSM cells were lipofected with (A) no DNA, 200 nM of sense p21^{Waf1/Cip1} or antisense p21^{Waf1/Cip1}; and (B) 200 nM of random sequence control oligodeoxynucleotide or antisense p21^{Waf1/Cip1}; and various concentrations of sense p21^{Wafl/Cip1} or antisense p21^{Waf1/Cip1} in (C) A10 and (D) bovine VSM cells. The experiments shown are representative of two to three separate experiments.

FIG. 2 is a bar graph showing VSM cell proliferation is inhibited by antisense p21^{Waf1/Cip1} oligodeoxynucleotide, as described in Example I, infra. A10 VSM cells were transfected as in FIG. 1C. Cell numbers are expressed as mean ±s.e.m. of three wells per data point.

FIGS. 3A and B show photographs of antisense oligodeoxynucleotides which were successfully transfected into VSM cells, as described in Example I, infra. FITC-tagged p21^{Waf1/Cip}1 antisense oligodeoxynucleotide was lipofected into A10 VSM cells and the same microscopic field was visualized by (A) visible and (B) fluorescence light at 40×.

FIG. 4 depicts a gel showing PMA induces p21^{Waf1/Cip1} in VSM cells, as described in Example I, infra. Non-transfected A10 VSM cells were stimulated with PMA (100 ng/ml) for the times indicated and Western blotted with p21^{Waf1/Cip1} antibody. The experiment shown is representative of three separate experiments.

FIG. 5 depicts a gel showing PMA stimulated CKI induction is blunted in antisense p21^{Waf1/Cip}1 transfected cells, as described in Example I, infra. A10 VSM cells were transfected with antisense p21^{Waf1/Cip}1 or sense p21^{Waf1/Cip}1 oligodeoxynucleotide as in FIG. 1. After overnight incubation, the cells were stimulated with PMA for the times indicated, lysed, and the lysates were Western blotted with p21^{Waf1/Cip}1 antibody. The experiment shown is representative of three separate experiments.

FIGS. 6A and B are gels showing antisense p21^{Waf1/Cip}1 inhibition of p21^{Waf1/Cip1}, as described in Example I, infra. A10 VSM Cells were lipofected with antisense p21^{Waf1/Cip}1 or sense p21^{Waf1/Cip}1 oligonucleotides, as in FIG. 1. (A) antibody to p21^{Waf1/Cip1}; antibody to p27^{Kip1} (B) α-actin antibody. The experiments shown are each representative of two separate experiments.

FIG. 7 depicts gels showing antisense p21^{Waf1/Cip1} inhibits cyclin D1/cdk4, but not cyclin E/cdk2, association, as described in Example I, infra. A10 VSM cells were transfected with antisense or sense p21^{Waf1/Cip1} as in FIG. 1. The arrowhead indicates cdk 4 or cdk 2. The band to the right of each blot is the 2 hours sense lysate immunoprecipitated and immunoblotted with cdk 2 or cdk 4 as a positive control. The thick band at the top of each blot is the heavy chain of IgG from the immunoprecipitation. The experiments shown are each representative of two separate experiments.

FIG. 8 is a gel depicting PMA stimulated CKI induction is blunted in antisense p21^{Waf1/Cip}1 transfected cells, as described in Example II, infra. The experiments shown are representative of two separate experiments.

FIG. 9A-D are bar graphs showing Antisense p21^{Waf1/Cip}1 oligodeoxynucleotide has no significant effect DNA synthesis in PMA-inhibitable A431 cells, as described in Example II, infra. A431 or A10 cells were lipofected with from 0 to 400 nM of sense p21^{Waf1/Cip1} or antisense p21^{Waf1/Cip1}. A431 cells were placed in serum-free medium overnight and then stimulated with (A) PDGF-BB (30 ng/ml), (B) 10% serum-containing medium, or (C) PDGF-BB or PMA (100 ng/ml) for another 8 hours before [³H]-thymidine was added for overnight incubation. (D) A10 VSM cells were transfected with sense and antisense p21^{Waf1/Cip1} oligonucleotides as above and treated similarly to (B). DNA synthesis was assessed by [³H]-thymidine incorporation and is expressed as mean ±s.e.m. of three wells per data point. The absolute counts differ between experiments due to different confluency of the cells. The experiments shown are representative of two separate experiments.

FIG. 10 is a Western blot showing levels of p53 protein were not altered in A431 cells as compared to A10 VSM cells, as described in Example II, infra. The experiment shown is representative of two separate experiments.

FIG. 11 is a Western blot showing Serum-induced hyperphosphorylation of Rb was not altered in A431 cells, as described in Example II, infra. The experiment shown is representative of two separate experiments.

FIG. 12 is a gel showing Antisense p21^{Waf1/Cip}1 altered cyclin D1/cdk4 association in A431 cells, as described in Example II, infra. The arrowhead indicates cdk4 (top blot) or cdk2 (bottom blot). The band to the right of each blot is the 2 hours sense lysate immunoprecipitated and immunoblotted with cdk2 or cdk4 as a positive control. The thick band at the top of each blot is the heavy chain of IgG from the immunoprecipitation. The experiments shown are each representative of two separate experiments.

FIG. 13 is a bar graph showing antisense p21^{Waf1/Cip1} oligodeoxynucleotide potentiates the cell cycle inhibitory (and presumably killing) effect of γ-irradiation on VSM cells exposed to serum, as described in Example III infra. The experiment shown is representative of two separate experiments.

FIGS. 14A and B illustrate how ionizing radiation inhibits DNA synthesis in VSM, but not A431, cells, as described in Example III, infra. Confluent (A) A10 VSM or (B) A431 cells were subjected to one of the following culture conditions: left in 10%-serum containing medium (continuous S); placed in serum-free medium the day of the experiment and left under those conditions for 48 hours (continuous SF); placed in serum-free medium for 24 hours and then stimulated with 10%-serum (SF→S) or PDGF-BB (30 ng/ml) (SF→PDGF-BB). All cells were irradiated with 8 Gy; when agonist was added, it was added 30 min after radiation. Six hours after agonist addition (where indicated), [³H]-thymidine (1 μCi/ml) was added to the medium overnight and DNA synthesis was assessed. Data is expressed as mean ±s.e.m. of three wells per data point. * indicates p<0.05 compared to control (random sequence oligonucleotide). The experiments shown are representative of two separate experiments.

FIGS. 15A and B show the induction of p21 in VSM cells by ionizing radiation is blunted by antisense oligonucleotide to p21, as described in Example III, infra. Confluent VSM cells were transfected with antisense oligonucleotide to p21 or random sequence control oligonucleotide as described in Materials and Methods. (A) Non-transfected cells were exposed to ionizing radiation (12 Gy), lysed at the indicated times after exposure, and Western blotted with p21 antibody. (B) Cells transfected with the indicated oligonucleotides were treated similarly to (A). The arrowhead shows the band corresponding to p21. The experiments shown are representative of two separate experiments.

FIG. 16 depicts the induction of p21 in VSM cells by Adriamycin is blunted by antisense oligonucleotide to p21, as described in Example III, infra. The arrowhead shows the band corresponding to p21. The experiments shown are representative of two separate experiments.

FIG. 17 illustrates how the antisense oligonucleotide to p21 potentiates radiation-induced VSM cell cycle arrest, as described in Example III, infra. The experiment shown is representative of two separate experiments. *,#p<0.05 compared to control; +p<0.05 compared to random sequence oligonucleotide.

FIG. 18 demonstrates how the antisense oligonucleotide to p21 potentiates Adriamycin-induced VSM cell cycle arrest, as described in Example III, infra. The experiment shown is representative of two separate experiments. *,#p<0.05 compared to control; +p<0.05 compared to random sequence oligonucleotide.

FIGS. 19A and B depict how Caspase-3 is activated by antisense oligonucleotide to p21 but not early after radiation or Adriamycin, as described in Example III, infra. Confluent VSM cells were transfected with oligonucleotide in the concentration indicated, left in serum-containing media overnight, and exposed to (A) ionizing radiation (12 Gy) or (B) Adriamycin where indicated. 4 hours later, activation of caspase-3 was assessed by Western blotting. The arrowhead indicates the cleavage product of caspase-3 signifying its processing as an early step in apoptosis. Wortmannin (wort) is a positive control for apoptosis. The arrowhead shows the band corresponding to the cleavage product of caspase-3. The experiment shown is representative of two separate experiments.

FIG. 20A-D show how the antisense oligonucleotide to p21 induces VSM cell apoptosis, as described in Example III, infra. VSM cells were grown on glass cover slips and transfected with (A,B) random sequence control oligonucleotide to p21 or (C,D) antisense oligonucleotide. 24 hours later, the cells were fixed and stained in situ with Hoechst 33258. Representative microscopic fields were photographed under (A,C) visual or (B,D) UV light at 40×.

FIG. 21 shows that TGF-β decreases mitogenesis in serum-starved VSM cells, as described in Example IV, infra. * indicates significance difference from control (no TGF-β). The experiment shown is representative of three separate experiments.

FIG. 22 shows how TGF-β decreases 10% serum-stimulated mitogenesis in VSM cells, as described in Example IV, infra. The * indicates a significant difference from serum alone. The experiment shown is representative of two separate experiments.

FIG. 23 demonstrates the transfection of VSM cells with antisense p21 oligodeoxynucleotide specifically reduces p21 protein level in cells, as described in Example IV, infra. The experiment shown is representative of at least three separate experiments.

FIG. 24 illustrates that TGF-β remains inhibitory in VSM cells transfected with antisense p21 oligodeoxynucleotide cells, as described in Example IV, infra. A10 VSM cells were grown to confluence, transfected as described with either antisense (solid bars) or control (hatched bars) oligodeoxynucleotide, and serum-starved overnight. Subsequently, the cells were treated with 10% serum containing medium and/or TGF-β at the indicated concentrations (in ng/ml), and DNA synthesis assessed as in FIG. 22; absolute counts differ slightly from other experiments due to differences in starting confluency of the cells. * indicates significance difference from serum alone. The experiment shown is representative of two separate experiments.

FIG. 25 shows that the antisense p21 oligodeoxynucleotide decreases TGF-β-mediated laminin production and secretion cells, as described in Example IV, infra. The experiment shown is representative of three separate experiments.

FIG. 26 demonstrates how the antisense p21 oligodeoxynucleotide decreases TGF-β-mediated fibronectin production and secretion cells, as described in Example IV, infra. The experiment shown is representative of three separate experiments.

FIG. 27 illustrates that expression of p21^{Waf1/Cip1} protein is elevated in breast tumor tissue, as described in Example VI, infra. Samples were obtained from eight human breast tumors and corresponding normal tissue. A. Shows a Western blot containing protein samples from various breast tumor tissues. B. Shows a Western blot containing protein samples from various breast tumor tissues and HeLa cells as a control. C. Shows immunohisotchmeical analysis of breast tumor tissues.

FIG. 28 demonstrates that production of P13K-related proteins are increased in p21-overexpressing tumors, as described in Example VI, infra. A. Shows a Western blot containing protein samples from various breast tumor tissues and detection of p85. B. Shows a Western blot containing protein samples from various breast tumor tissues and detection of PTEN.

FIG. 29 shows that p21 is expressed at high levels in serum-cultured, breast tumor cell lines, MCF7 (adenocarcinoma) and T47D (ductal carcinoma) cells as shown in Example VI, infra.

FIG. 30 illustrates that serum-starved, breast cancer cells transfected with antisense p21 ODN show reduced levels of p21 as shown in Example VI, infra. A. Shows a Western blot containing protein samples from breast tumor cell line T47D. B. Shows a Western blot containing protein samples from breast tumor cell line MCF7. C. Shows immunochemistry of transfected T47D and MCF7 cell lines.

FIG. 31 shows morphological changes indicative of apoptosis in breast tumor cell lines, MCF7 and T47D, lipofected with p21, as described in Example VI, infra.

FIG. 32 shows PARP cleavage in breast tumor cell lines lipofected with antisense p21, as described in Example VI, infra. A. Shows a Western blot containing protein samples from T47D. B. Shows a Western blot containing protein samples from MCF7.

FIG. 33 is a bar graph showing colorimetric analysis of caspase-3 cleavage products in a breast tumor cell line, T47D lipofected with antisense p21, as described in Example VI, infra.

FIG. 34 is a bar graph showing [³H] thymidine incorporation in breast tumor cell lines, T47D and MCF7, lipofected with antisense p21, as described in Example VI, infra.

FIG. 35: Human p21 antisense oligodeoxynucleotide (ODN) sequence.

FIG. 36: Randomly scrambled sequence of control oligodeoxynucleotide (ODN).

FIG. 37 shows immunohistochemical analysis of rat aortic VSM cells, over-expressing p21-ΔNLS causes p21 to localize in the cytosol as described in Example VII, infra.

FIG. 38 shows Zn-responsiveness of the ΔNLS-p21 construct in rat aortic VSM cells, transfected with ΔNLS-p21, as described in Example VII, infra. A. The Western blot contains protein samples from VSM cells transfected with ΔNLS-p21 (left) or full-length p21 (right). B. The Western blot contains protein samples from VSM cells transfected with vector-p21 (left) or ΔNLS-p21 (right).

FIG. 39 shows bar graphs showing [³H] thymidine incorporation of rat aortic VSM cells transfected with p21 or ΔNLSp21, under Zn induction, as described in Example VII, infra. A. VSM cells transfected with a ΔNLSp21 vector. B. VSM cells transfected with full-length p21. C. VSM cells tranfected with an empty vector.

FIG. 40 is a bar graph showing [³H]thymidine incorporation in rat aortic VSM cells transfected with pMTCB6-ΔNLSp21, clones 1, 7 or 9, as described in Example VII, infra.

FIG. 41 shows bar graphs of rat aortic VSM cells transfected with ΔNLSp21 or full-length p21, and treated with PDGF-BB. A. Cells tranfected with ΔNLSp21. B. Cell transfected with full-length p21.

### DETAILED DESCRIPTION OF THE INVENTION

The present invention is based on the surprising discovery that p21^{Waf1/Cip1} protein serves a permissive role in PDGF-mediated cell growth and proliferation, such that its presence is required for the mitogenic effect of this growth factor, for example, in VSM cells. Therefore, successful therapy and prevention of abnormal growth and proliferation of cells must take into account p21^{Waf1/Cip1} activity or function to effectively combat proliferation of cells that result in disease states.

The methods and compositions of the invention can be used to treat a variety of diseases associated with abnormal cell growth and proliferation, including, but not limited to, atherosclerosis, angioplasty restenosis, renal mesangial cell proliferation and cancer, as well as preventing the VSM cell proliferation seen after coronary angioplasty, and may additionally be useful in cancer treatment as a sensitizer to chemotherapy and/or radiation (Mueller et al., 2000 Cancer Res. 2000. 60.(1):156.-63., 60, p. 156-163; and Wouters et al., 1997 Cancer Res., 57, p. 4703-4706). The methods may also be used to prevent plaques or tumors from forming.

The methods of the invention include regulation of cell growth mediated by CDKs by inhibiting p21^{Waf1/Cip1}, using a p21^{Waf1/Cip}1 inhibitory agent to suppress abnormal cell growth and proliferation in VSM cells and other cells, including tumors (Mueller et al., 2000 Cancer Res.2000.Jan.1;60.(1):156.-63, 60, p. 156-163; and Wouters et al., 1997 Cancer Res., 57, p. 4703-4706).

Definitions

As used herein a “p21^{Waf1/Cip1}” and “p21” are used interchangeably.

Inhibition of cell growth and proliferation, as used herein, means an effective decrease in the number of cells treated with the compound of the invention e.g. antisense oligonucleotide of p21, as compared to non-treated cells.

As used herein a “p21^{Waf1/Cip1} inhibitory agent” is an agent that directly or indirectly inhibits activity of p21^{Waf1/Cip1}. A direct inhibitory agent, for example, is an antibody or antagonist that binds to and inhibits the activity of p21^{Waf1/Cip1}, soluble forms and fragments thereof having p21^{Waf1/Cip1}-binding activity, and new p21^{Waf1/Cip}1 antagonists developed using well known methods for drug discovery as described herein and in the art. If the agent is p21^{Waf1/Cip1} specific (i.e. a direct inhibitory agent), it prevents proliferation of cells at the site of abnormal proliferation, such as the heart or the vascular system. An indirect inhibitor, such as an antisense oligonucleotide of p21^{Waf1/Cip1}, inhibits the synthesis or secretion of p21^{Waf1/Cip1}, by binding to the nucleic acid sequence of p21^{Waf1/Cip}1 and/or inhibits the expression (i.e. transcription or translation) of p21^{Waf1/Cip1}, thereby reducing the amount of p21^{Waf1/Cip1} produced, or sequestering it away from its target protein.

Methods and Compositions of the Invention

The present invention provides methods and compositions to treat diseases associated with abnormal cell proliferation, by inhibiting the expression or activity of p21^{Waf1/cip}1 In one embodiment, a p21^{Waf1/Cip}1 inhibitory agent is administered to a subject at risk for such diseases, for example atherosclerosis to prevent abnormal proliferation. Such individuals can be prescreened using known medical procedures such as serum cholesterol measurements, history of premature heart disease, and invasive and non-invasive measurements of cardiac ischemia.

Included within the scope of p21^{Waf1/Cip1} indirect inhibitors of the invention are nucleic acids, including antisense oligonucleotides, that block the expression of p21^{Waf1/Cip1} genes within cells, by binding a complementary messenger RNA (mRNA) and preventing its translation (Wagner, Nature 372:332-335 (1994); and Crooke and Lebleu, Antisense Research and Applications, CRC Press, Boca Raton (1993)). Inhibition of gene expression may be measured by determining the degradation of the target RNA. Antisense DNA and RNA can be prepared by methods known in the art for synthesis of RNA including chemical synthesis such as solid phase phosphoramidite chemical synthesis or in vitro and in vivo transcription of DNA sequences encoding the antisense RNA molecule.

The antisense oligonucleotides comprise a nucleotide sequence which is complementary to a nucleotide sequence encoding p21^{Waf1/Cip}1 protein. In one embodiment, the antisense oligonucleotide comprises the nucleotide sequence shown in SEQ ID NO: 1. In another embodiment, the antisense oligonucleotide is an RNA, DNA, or RNA/DNA hybrid molecule.

In another embodiment, the antisense oligonucleotide is a derivative nucleic acid molecule. Derivative molecules include peptide nucleic acids (PNAs), and non-nucleic acid molecules including phosphorothioate, phosphotriester, phosphoramidate, and methylphosphonate molecules, that bind to single-stranded DNA or RNA in a base pair-dependent manner (Zamecnik, P. C., et al., 1978 Proc. Natl. Acad. Sci. 75:280284; Goodchild, P. C., et al., 1986 Proc. Natl. Acad. Sci. 83:4143-4146). Peptide nucleic acid molecules comprise a nucleic acid oligomer to which an amino acid residue, such as lysine, and an amino group have been added. These molecules stop transcript elongation by binding to their complementary (template) strand of nucleic acid (Nielsen, P. E., et al., 1993 Anticancer Drug Des 8:53-63). Reviews of methods for synthesis of DNA, RNA, and their analogues can be found in: Oligonucleotides and Analogues, eds. F. Eckstein, 1991, IRL Press, New York; Oligonucleotide Synthesis, ed. M. J. Gait, 1984, IRL Press, Oxford, England. Additionally, methods for antisense RNA technology are described in U.S. Pat. Nos. 5,194,428 and 5,110,802. A skilled artisan can readily obtain these classes of nucleic acid molecules using the herein described Siglec-BMS polynucleotide sequences, see for example Innovative and Perspectives in Solid Phase Synthesis (1992) Egholm, et al. pp 325-328 or U.S. Pat. No. 5,539,082.

The present invention provides antisense molecules which bind a nucleic acid molecule encoding a p21^{Waf1/Cip1} protein. The antisense molecules of the present invention can inhibit production of a p21^{Waf1/Cip1} protein in a cell, or reduce the level of a p21^{Waf1/Cip1} protein in a cell, by binding a nucleic acid molecule encoding a p21^{Waf1/Cip1} protein. The antisense molecule can bind a single-stranded DNA molecule encoding p21^{Waf1/Cip1}, or bind one strand of a double-stranded DNA molecule, thereby inhibiting transcription of the DNA molecule. The antisense molecule can bind a nascent or non-nascent RNA molecule encoding p21^{Waf1//Cip1}, thereby inhibiting transcription and/or translation of the RNA molecule.

The antisense DNA sequences may be incorporated into vectors with RNA polymerase promoters such as the T7 or SP6 polymerase promoters. Alternatively, antisense cDNA constructs that synthesize antisense RNA constitutively or inducibly can be introduced into cell lines. The potency of antisense oligonucleotides for inhibiting p21^{Waf1/Cip1} may be enhanced using various methods including: 1) addition of polylysine (Leonetti et al., Bioconj. Biochem. 1:149-153 (1990)); 2) encapsulation into antibody targeted liposomes (Leonetti et al., Proc. Natl. Acad. Sci. USA 87:2448-2451 (1990) and Zelphati et al., Antisense Research and Development 3:323-338 (1993)); 3) nanoparticles (Rajaonarivony et al., J. Pharmaceutical Sciences 82:912-917 (1993) and Haensler and Szoka, Bioconj. Chem. 4:372-379 (1993)), 4) the use of cationic acid liposomes (Felgner et al., Proc. Natl. Acad. Sci. USA 84:7413-7417 (1987); Capaccioli et al., Biochem. Biophys. Res. Commun. 197:818-825 (1993); Boutorine and Kostina, Biochimie 75:35-41 (1993); Zhu et al., Science 261:209-211 (1993); Bennett et al., Molec. Pharmac. 41:1023-1033 (1992) and Wagner, Science 280:1510-1513 (1993)); 5) Sendai virus derived liposomes (Compagnon et al., Exper. Cell Res. 200:333-338 (1992) and Morishita et al., Proc. Natl. Acad. Sci. USA 90:8474-8478 (1993)), to deliver the oligonucleotides into cells; and (6) the conjugation of the antisense oligonucleotides to a fusogenic peptide, e.g. derived from an influenza hemagglutinin envelope protein (Bongartz et al., Nucleic Acids Res. 22(22):4681-4688 (1994)).

Also included within the scope of p21 direct inhibitors of the invention are antagonists which bind to p21. The term “antagonists,” as it is used herein, refers to a molecule which, when bound to p21, decreases the amount or the duration of the effect of the biological activity of p21. Antagonists may include proteins, nucleic acids, carbohydrates, antibodies, or any other molecules which modulate the cell proliferation effects of p21. Suitable p21 antagonists can be readily determined using methods known in the art to screen candidate agent molecules for binding to p21, such as assays for detecting the ability of a candidate agent to measure CDK immunoprecipitation and check effect of immunoprecipitated CDK on Rb phosphorylation (Sherr, C. J and Roberts J. M. Genes and Development, 13, 1501-1512 (1999)).

Direct inhibitors such as antibodies of the invention include polyclonal, monoclonal, chimeric, fragments, and humanized antibodies, that bind to p21 proteins or fragments of p21 proteins thereof. The most preferred antibodies will selectively bind to p21 proteins and will not bind (or will bind weakly) to non-p21 proteins. These antibodies can be from any source, e.g., rabbit, sheep, rat, dog, cat, pig, horse, mouse and human.

As will be understood by those skilled in the art, the regions or epitopes of a p21 protein to which an antibody is directed may vary with the intended application. For example, antibodies intended for use in an immunoassay for the detection of membrane-bound p21 on viable cells should be directed to an accessible epitope. The p21 proteins represents potential markers for screening, diagnosis, prognosis, and follow-up assays and imaging methods. In addition, based on the discoveries described herein, p21 proteins may be excellent targets for therapeutic methods such as targeted antibody therapy, immunotherapy, and gene therapy to treat conditions associated with the presence or absence of p21 proteins. Antibodies that recognize other epitopes may be useful for the identification of p21 within damaged or dying cells, for the detection of secreted p21 proteins or fragments thereof. Additionally, some of the antibodies of the invention may be internalizing antibodies, which internalize (e.g., enter) into the cell upon or after binding. Internalizing antibodies are useful for inhibiting cell growth and/or inducing cell death.

The invention includes a monoclonal antibody, the antigen-binding region of which competitively inhibits the immunospecific binding of any of the monoclonal antibodies of the invention to its target antigen. Further, the invention provides recombinant proteins comprising the antigen-binding region of any the anti-p21 monoclonal antibodies of the invention.

The invention also encompasses antibody fragments that specifically recognize a p21 protein or a fragment thereof. As used herein, an antibody fragment is defined as at least a portion of the variable region of the immunoglobulin molecule that binds to its target, i.e., the antigen binding region. Some of the constant region of the immunoglobulin may be included. Fragments of the monoclonal antibodies or the polyclonal antisera include Fab, F(ab′)₂, Fv fragments, single-chain antibodies, and fusion proteins which include the immunologically significant portion (i.e., a portion that recognizes and binds p21).

The chimeric antibodies of the invention are immunoglobulin molecules that comprise at least two antibody portions from different species, for example a human and non-human portion. Chimeric antibodies are useful, as they are less likely to be antigenic to a human subject than antibodies with non-human constant regions and variable regions. The antigen combining region (variable region) of a chimeric antibody can be derived from a non-human source (e.g. murine) and the constant region of the chimeric antibody, which confers biological effector function to the immunoglobulin, can be derived from a human source (Morrison et al., 1985 Proc. Natl. Acad. Sci. U.S.A. 81:6851; Takeda et al., 1985 Nature 314:452; Cabilly et al., U.S. Pat. No. 4,816,567; Boss et al., U.S. Pat. No. 4,816,397). The chimeric antibody may have the antigen binding specificity of the non-human antibody molecule and the effector function conferred by the human antibody molecule.

The chimeric antibodies of the present invention also comprise antibodies which are chimeric proteins, having several distinct antigen binding specificities (e.g. anti-TNP: Boulianne et al., 1984 Nature 312:643; and anti-tumor antigens: Sahagan et al., 1986 J. Immunol. 137:1066). The invention also provides chimeric proteins having different effector functions (Neuberger et al., 1984 Nature 312:604), immunoglobulin constant regions from another species and constant regions of another immunoglobulin chain (Sharon et al., 1984 Nature 309:364); Tan et al., 1985 J. Immunol. 135:3565-3567). Additional procedures for modifying antibody molecules and for producing chimeric antibody molecules using homologous recombination to target gene modification have been described (Fell et al., 1989 Proc. Natl. Acad. Sci. USA 86:8507-8511).

Humanized antibodies directed against p21 proteins are also useful. As used herein, a humanized p21 antibody is an immunoglobulin molecule which is capable of binding to a p21 protein. A humanized p21 antibody includes variable regions having substantially the amino acid sequence of a human immunoglobulin and the hyper-variable region having substantially the amino acid sequence of non-human immunoglobulin. Humanized antibodies can be made according to several methods known in the art (Teng et al., 1983 Proc. Natl. Acad. Sci. U.S.A. 80:7308-7312; Kozbor et al., 1983 Immunology Today 4:7279; Olsson et al., 1982 Meth. Enzymol. 92:3-16).

Various methods for the preparation of antibodies are well known in the art. For example, antibodies may be prepared by immunizing a suitable mammalian host with an immunogen such as an isolated p21 protein, peptide, fragment, or an immunoconjugated form of p21 protein (Harlow 1989, in: Antibodies, Cold Spring Harbor Press, New York). In addition, fusion proteins of p21 may also be used as immunogens, such as a P21 fused to -GST-, -human Ig, or His-tagged fusion proteins. Cells expressing or overexpressing p21 proteins may also be used for immunizations. Similarly, any cell engineered to express p21 proteins may be used. This strategy may result in the production of monoclonal antibodies with enhanced capacities for recognizing endogenous p21 proteins (Harlow and Lane, 1988, in: Antibodies: A Laboratory Manual. Cold Spring Harbor Press).

The amino acid sequence of p21 proteins, and fragments thereof, may be used to select specific regions of the p21 proteins for generating antibodies. For example, hydrophobicity and hydrophilicity analyses of the p21 amino acid sequence may be used to identify hydrophilic regions in the p21 protein structure. Regions of the p21 protein that show immunogenic structure, as well as other regions and domains, can readily be identified using various other methods known in the art (Rost, B., and Sander, C. 1994 Protein 19:55-72), such as Chou-Fasman, Garnier-Robson, Kyte-Doolittle, Eisenberg, Karplus-Schultz or Jameson-Wolf analysis. Fragments including these residues are particularly suited in generating anti-p21 antibodies.

Methods for preparing a protein for use as an immunogen and for preparing immunogenic conjugates of a protein with a carrier such as BSA, KLH, or other carrier proteins are well known in the art. Techniques for conjugating or joining therapeutic agents to antibodies are well known (Arnon et al., “Monoclonal Antibodies For Immunotargeting Of Drugs In Cancer Therapy”, in: Monoclonal Antibodies And Cancer Therapy, Reisfeld et al. (eds.), pp. 243-56 (Alan R. Liss, Inc. 1985); Hellstrom et al., “Antibodies For Drug Delivery”, in: Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in: Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982); Sodee et al., 1997, Clin. Nuc. Med. 21: 759-766). In some circumstances, direct conjugation using, for example, carbodiimide reagents may be used; in other instances linking reagents such as those supplied by Pierce Chemical Co., Rockford, Ill., may be effective.

Administration of a p21 immunogen is conducted generally by injection over a suitable time period and with use of a suitable adjuvant, as is generally understood in the art. During the immunization schedule, titers of antibodies can be taken to determine adequacy of antibody formation.

While the polyclonal antisera produced in this way may be satisfactory for some applications, for pharmaceutical compositions, monoclonal antibody preparations are preferred. Immortalized cell lines which secrete a desired monoclonal antibody may be prepared using the standard method of Kohler and Milstein (Nature 256: 495-497) or modifications which effect immortalization of lymphocytes or spleen cells, as is generally known. The immortalized cell lines secreting the desired antibodies are screened by immunoassay in which the antigen is the p21 protein or a fragment thereof. When the appropriate immortalized cell culture secreting the desired antibody is identified, the cells can be cultured either in vitro or by production in ascites fluid. The desired monoclonal antibodies are then recovered from the culture supernatant or from the ascites supernatant.

The antibodies or fragments may also be produced by recombinant means. The antibody regions that bind specifically to the desired regions of the p21 protein can also be produced in the context of chimeric or CDR grafted antibodies of multiple species origin.

The antibodies of the invention bind specifically to polypeptides having p21 sequences. In one embodiment, the p21 antibodies specifically bind to the extracellular domain of a p21 protein. In other embodiments, the antibodies of the invention specifically bind to other domains of a p21 protein or precursor, for example the antibodies bind to the cytoplasmic domain of p2l proteins.

Additionally, some of the antibodies of the invention are internalizing antibodies, i.e., the antibodies are internalized into the cell upon or after binding (Liu, H. et al., Cancer Res. 1998, 58, 4055-4060).

The indirect p21^{Waf1/Cip}1 inhibitory agent can be a small peptide which binds Akt. In one embodiment, the small peptide is an arginine-rich peptide molecule. In another embodiment, the small peptide comprises a consensus amino acid sequence.

The methods of the invention comprise introducing the direct or indirect p21^{Waf1/Cip}1 inhibitory agents so as to inhibit production of a p21^{Waf1/Cip}1 protein and/or to inhibit the activity of a p21^{Waf1/Cip}1 protein. In one embodiment, the methods comprise contacting a cell with, or introducing into a cell, an inhibitory agent that inhibits production of the p21^{Waf1/Cip}1 protein. In another embodiment, the methods comprise contacting a cell with, or introducing into a cell, an inhibitory agent that inhibits the activity of the p21^{Waf1/Cip1} protein. For example, an anti-p21 mAb can be introduced into a subject to contact p21 positive cells to inhibit the activity of p2l and decrease the proliferation of cells.

In addition, the invention provides a process for the production of vaccines using p21 protein and a vaccine for treating cyclin-dependent kinase-mediated cell growth and proliferation. The vaccines contain a p21 protein, or partial sequences thereof, which is carrier-bound if desired, as an immunogen in a pharmacologically effective dose, and in a pharmaceutically acceptable formulation.

The production of these vaccines can be carried out according to known methods. However, the p21 proteins are preferably first lyophilized and subsequently suspended, if desired with addition of auxiliary substances.

Vaccination with these vaccines or combinations of vaccines according to the present invention can be carried out according to methods familiar to one skilled in the art (e.g. intradermally, intramuscularly, intraperitoneally, intravenously, subcutaneously or intranasally).

For intramuscular or subcutaneous administration, the vaccine can, for example, be suspended in physiological saline. For an intranasal or intraoccular application, the vaccine can, e.g., be used in the form of a spray or an aqueous solution. For a local, e.g. oral, administration, it is often necessary to temporarily protect the immunogens against inactivation, for example against proteolytic enzymes in the cavity of the mouth or in the stomach. Such temporary protection can be achieved by encapsulating the immunogens. This encapsulation can be carried out by coating with a protective agent (microencapsulation) or by embedding a multitude of immunogens according to the present invention in a protective carrier (macroencapsulation).

The encapsulation material can be semipermeable or become semipermeable when introduced into the human or animal body. A biologically degradable substance is usually used as a carrier for the encapsulation.

The present invention provides methods for inhibiting cell proliferation mediated by a cyclin-dependent kinase. In one embodiment, the methods comprise contacting a p21 protein with, or introducing into a cell, an effective amount of an inhibitory agent. For example, the inhibitory agent can bind a nucleic acid molecule encoding a p21^{Waf1/cip}1 protein or binds a p21^{Waf1/cip1} protein, thereby reducing the level of a p21^{Waf1/cip1} protein in the cell or inhibiting the activity of a p21^{Waf1/cip}1 protein in the cell. By reducing the level of a p21^{Waf1/cip1} protein in the cell or inhibiting the activity of a p21^{Waf1/cip}1 protein in the cell, the inhibitory agent inhibits cell proliferation. In one embodiment, the cyclin-dependent kinase is a cdk2 or cdk4. The cdk2 or cdk4 can part of a complex comprising a cyclin A or cyclin D1 protein.

The present invention also provides methods for modulating cell proliferation (e.g., increasing or inhibiting). In one embodiment, the methods comprise contacting a cell with, or introducing into a cell, an effective amount of an inhibitory agent. For example, the inhibitory agent can be a nucleic acid molecule encoding a p21^{Waf1/cip}1 protein, or can be a p21^{Waf1/cip1} protein, having a motif that directs nuclear or cytosolic localization of the p21^{Waf1/cip1} protein. These localization motifs are previously described (Winters, Z. E., Hunt, N. C., Bradburn, M. J., Royds, J. A., Turley, H., Harris, A. L., and Norbury, C. J., Eur. J. Cancer, 37: 2405-2412, 2001; Li, Y., Dowbenko, D., and Lasky, L. A., J. Biol. Chem., 277: 11352-11361, 2002; Asada, M., Yamada, T., Ichijo, H., Delia, D., Miyazono, K., Fukumuro, K., and Mizutani, S., EMBO J., 18: 1223-1234, 1999; Zhou, B. P., Liao, Y., Xia, W., Spohn, B., Lee, M. H., and Hung, M. C., Nat. Cell Biol., 3: 245-252, 2001). In one embodiment, the encoded p21 protein or the p21 protein comprise the nuclear localization motif.

The present invention also provides methods for inhibiting DNA synthesis in a cell. In one embodiment, the methods comprise contacting a cell with, or introducing into a cell, an effective amount of an inhibitory agent. For example, the inhibitory agent can bind a nucleic acid molecule encoding a p21^{Waf1/cip1} protein or can bind a p21^{Waf1/cip1} protein, thereby reducing the level of a p21^{Waf1/cip1} protein in the cell or inhibiting the activity of a p21^{Waf/cip1} protein in the cell. By reducing the level of a p21^{Waf/cip1} protein in the cell or inhibiting the activity of a p21^{Waf/cip1} protein in the cell, the inhibitory agent inhibits DNA synthesis in the cell.

The present invention also provides methods for inhibiting formation of a complex comprising a p21^{Waf/cip1} protein and a cyclin dependent kinase. In one embodiment, the methods comprise contacting a cell with, or introducing into a cell, an effective amount of an inhibitory agent. For example, the inhibitory agent can bind a nucleic acid molecule encoding a p21^{Waf/cip1} protein or can bind a p21^{Waf/cip1} protein, thereby reducing the level of a p21^{Waf/cip1} protein in the cell or inhibiting the activity of a p21^{Waf/cip1} protein in the cell. By reducing the level of a p21^{Waf/cip1} protein in the cell or inhibiting the activity of a p21^{Waf/cip1} protein in the cell, the inhibitory agent inhibits formation of the complex. In one embodiment, the cyclin-dependent kinase is a cdk2 or cdk4. The complex can further comprise a cyclin A or cyclin D1 protein.

The present invention also provides methods for inducing cellular apoptosis. In one embodiment, the methods comprise contacting a cell with, or introducing into a cell, an effective amount of an inhibitory agent. For example, the inhibitory agent can bind a nucleic acid molecule encoding a p21^{Waf/cip1} protein or can bind a p21^{Waf/cip1} protein, thereby reducing the level of a p21^{Waf/cip1} protein in the cell or inhibiting the activity of a p21^{Waf/cip1} protein in the cell. By reducing the level of a p21^{Waf/cip1} protein in the cell or inhibiting the activity of a p21^{Waf/cip1} protein in the cell, the inhibitory agent induces cellular apoptosis. Cellular apoptosis can be detected by various methods, including observing cellular morphology, nuclear morphology, and/or detecting the presence of cleavage products of caspase-3 and/or PARP.

The present invention also provides methods for inhibiting production of a matrix protein by a cell. In one embodiment, the methods comprise contacting a cell with, or introducing into a cell, an effective amount of an inhibitory agent. For example, the inhibitory agent can bind a nucleic acid molecule encoding a p21^{Waf/cip1} protein or can bind a p21^{Waf/cip1} protein, thereby reducing the level of a p21^{Waf/cip1} protein in the cell or inhibiting the activity of a p21^{Waf/cip1} protein in the cell. By reducing the level of a p21^{Waf/cip1} protein in the cell or inhibiting the activity of a p21^{Waf/cip1} protein in the cell, the inhibitory agent inhibits production of a matrix protein by the cell. In one embodiment, the matrix protein is laminin or fibronectin.

Administration of Inhibitors

The direct or indirect p21^{Waf1/Cip}1 inhibitory agents may be administered to mammalian subjects, including: humans, monkeys, apes, dogs, cats, cows, horses, rabbits, mice and rats. The methods include administration by standard parenteral routes, such as subcutaneously, intravenously, intramuscularly, intracutaneously, intra-articularly, intrasynovially, intrathecally, periostally, or by oral routes. Alternative methods include, administration by implantable pump or continuous infusion, injection, or liposomes. Administration can be performed daily, weekly, monthly, every other month, quarterly or any other schedule of administration as a single dose injection or infusion, multiple doses, or in continuous dose form.

As is standard practice in the art, the direct or indirect p21^{Waf1/Cip1} inhibitory agents of the invention may be administered to the subject in any pharmaceutically acceptable carrier or adjuvant which is known to those of skill of the art. These carriers and adjuvants include, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances and polyethylene glycol.

The direct or indirect p21^{Waf1/Cip1} inhibitory agents may be administered to a subject in an amount and for a time sufficient to block the activity of p21^{Waf1/Cip1}, in the subject. The amount and time may also be sufficient to block p21^{Waf1/Cip1} positive cells direct or indirect p21^{Waf1/Cip1} inhibitory agents. The most effective mode of administration and dosage regimen for the inhibitors in the methods of the present invention depend on the severity of the abnormal proliferation of cells, the subject's health, previous medical history, age, weight, height, sex, response to treatment and the judgment of the treating physician. Therefore, the amount of inhibitors to be administered, as well as the number and timing of subsequent administrations are to be determined by a medical professional conducting therapy based on the response of the individual subject. Initially, such parameters are readily determined by skilled practitioners using appropriate testing in animal models for safety and efficacy, and in human subjects during clinical trials of candidate therapeutic inhibitor formulations. To determine if the amount administered is sufficient, the subject may be monitored for certain symptoms associated with the abnormal proliferation of cells.

Disruption of p53 (Bunz F, et al. J Clin Invest 104:263-269, 1999), and also of p21 (Wouters B G, et al. Cancer Res 57:4703-4706, 1997), sensitizes cancer cells to DNA damaging agents. Therefore, using the inhibitors of the invention in the methods of the invention, vascular cells may be rendered more sensitive to the effects of DNA damaging agents, such that targeted cells or tissues may be made more likely to become growth arrested and subsequently apoptotic, after p21 levels are attenuated. The invention also encompasses the use of the direct or indirect p21^{Waf1/Cip1} inhibitory agents of the invention together with other chemotherapeutic agents, such as adriamycin cisplatinum, carboplatin, vinblastine, vincristine, taxol, dactinomycin (actinomycin D), daunorubicin (daunomycin, rubidomycin), bleomycin, plicamycin (mithramycin), mitomycin (mitomycin C), methotrexate, cytarabine (AraC), azauridine, azaribine, fluorodeoxyuridine, deoxycoformycin, and mercaptopurine. In addition, those of skill in the art will appreciate that the compounds of the present invention can be used in conjunctive therapy with other known chemotherapeutic compounds.

The Examples, infra, include the demonstration that transfection of several lines of VSM cells with antisense oligodeoxynucleotide specific to p21^{Waf1/Cip1} correlated with decreased cyclin D1/cdk 4, but not cyclin E/cdk 2 association. The Examples also show a dose-dependent inhibition of PDGF-BB-stimulated DNA synthesis and cell proliferation. The Examples demonstrate that the presence of p21^{Waf1/Cip1} is required for growth factor-induced proliferation of VSM cells.

The following examples are presented to demonstrate the methods of the present invention and to assist one of ordinary skill in using the same. The examples are not intended in any way to otherwise limit the scope of the disclosure of the protection granted by Letters Patent granted hereon.

### EXAMPLE I

### p21^{Waf1/Cip1} Is Required For PDGF Induced Vascular Smooth Muscle Cell Proliferation

Materials: Human recombinant PDGF-BB was obtained from Upstate Biotechnology, Inc (UBI) (Lake Placid, N.Y.). Mouse monoclonal p21^{Waf1/Cip1} and p27^{Kip1} and cyclin D1, goat polyclonal cdk 2 and cdk 4, and rabbit polyclonal cyclin E antibodies were obtained from Santa Cruz Biotechnology (Santa Cruz, Calif.). Anti-goat horseradish peroxidase-conjugated IgG was obtained from BioRad (Richmond, Calif.). Lipofectin® was obtained form Life Technologies (Rockville, Md.). Reagents for the Enhanced Chemiluminescence system and [³H]thymidine were obtained from Amersham (Arlington Heights, Ill.). All other reagents, including mouse monoclonal α-actin antibody, were from Sigma (St. Louis, Mo.).

Cell culture, DNA synthesis, and proliferation assays: Cultures of both A10 and A7r5 rat aortic VSM cells were obtained from American Type Culture Collection (Rockville Md.). Bovine aortic smooth muscle cells were supplied by Martha O'Donnell (O'Donnell and Owen, 1986 Proc Natl. Acad. Sci.US.A., 83, p. 6132-6136). All of the cell lines were maintained as described (Weiss et al., 1998 Am. J. Physiol., 274, p. C1521-C1529) and were used between passages 15 and 25. The cells were growth-arrested by placing them in serum-free quiescence medium, exposed to growth factors as indicated, and [³H]thymidine incorporation assessed as previously described (Weiss and Nuccitelli, 1992a J. Biol. Chem., 267, p. 5608-5613). Cell proliferation was assessed by counting of adherent cells on 4 representative fields under 100× magnification in each of 3 wells per experimental condition.

Antisense transfections: Phosphorothioate antisense oligodeoxynucleotides were synthesized by Oligonucleotides Etc. (Wilsonville, Oreg.). The p21^{Waf1/Cip1} antisense vector was designed around the start codon of rat p21^{Waf1/Cip1}, with sequence 5′-GAC ATC ACC AGG ATC GGA CAT-3′ (SEQ. ID NO.:1). The sense p21^{Waf1/Cip1} sequence is 5′-ATG TCC GAT CCT GGT GAT GTC-3′ (SEQ. ID NO.:2). The scrambled random sequence control oligodeoxynucleotide was 5′-TGG ATC CGA CAT GTC AGA-3′ (SEQ. ID NO.:3). For the lipofection procedure, cells were grown to 90% confluence, the appropriate concentration of oligodeoxynucleotide was mixed with 6.6 μL of Lipofectin® per ml of Opti-MEM medium and was added to the cells for 4 hours at 37° C. The cells were washed and serum-free medium (without oligodeoxynucleotide) was added overnight, the media was changed in the morning and the cells were incubated in serum-free medium for the times indicated.

Western blots: Cells were grown to confluence in 6 cm culture dishes and serum deprived. After transfection and or treatment with appropriate agonist, the cells were washed with phosphate-buffered saline and lysed in lysis buffer and the supernatant was Western blotted as described (Weiss et al., 1998 Am. J. Physiol., 274, p. C1521-C1529).

1. Determination of the Dependence of G1-Phase Progression on p21^{Waf1/Cip1} in the VSM Cell Lines.

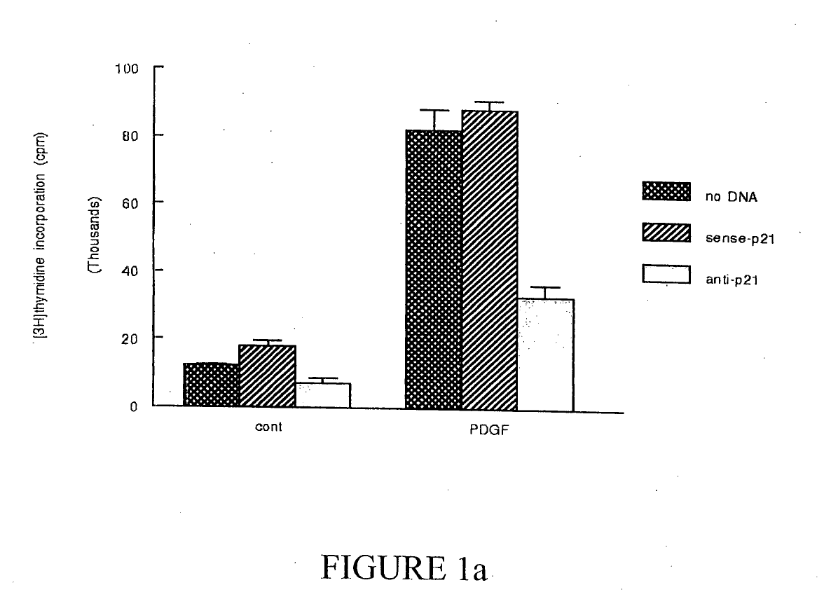

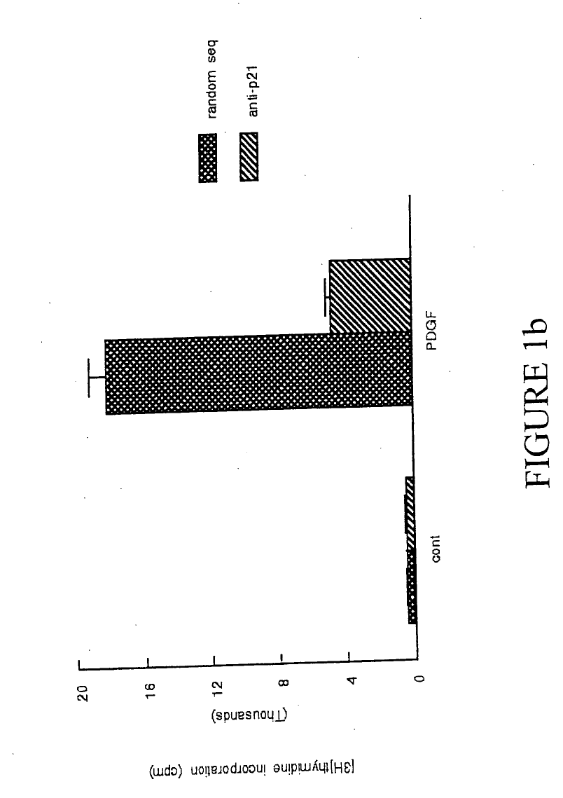

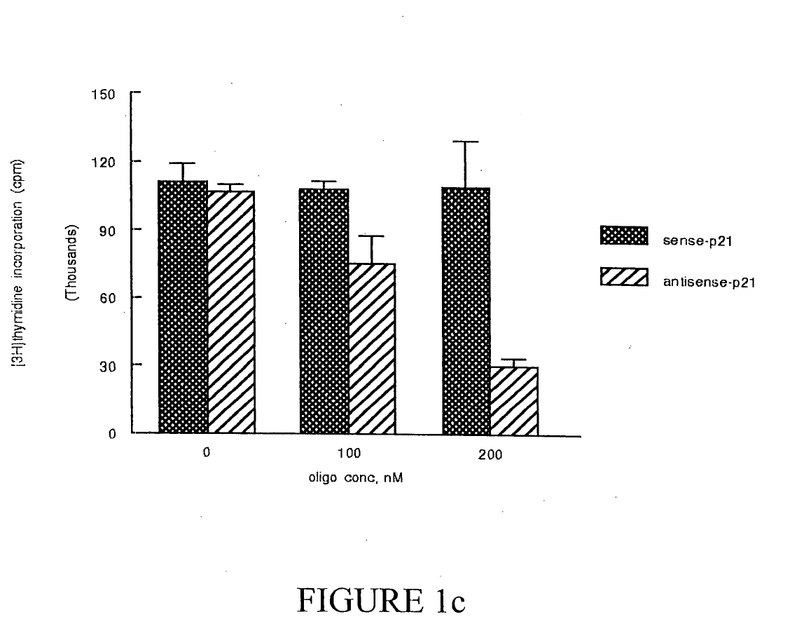

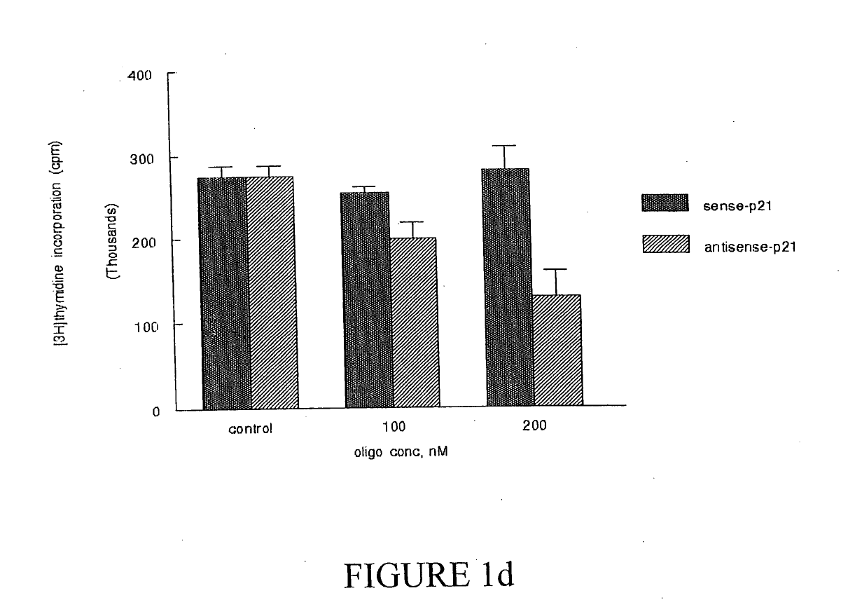

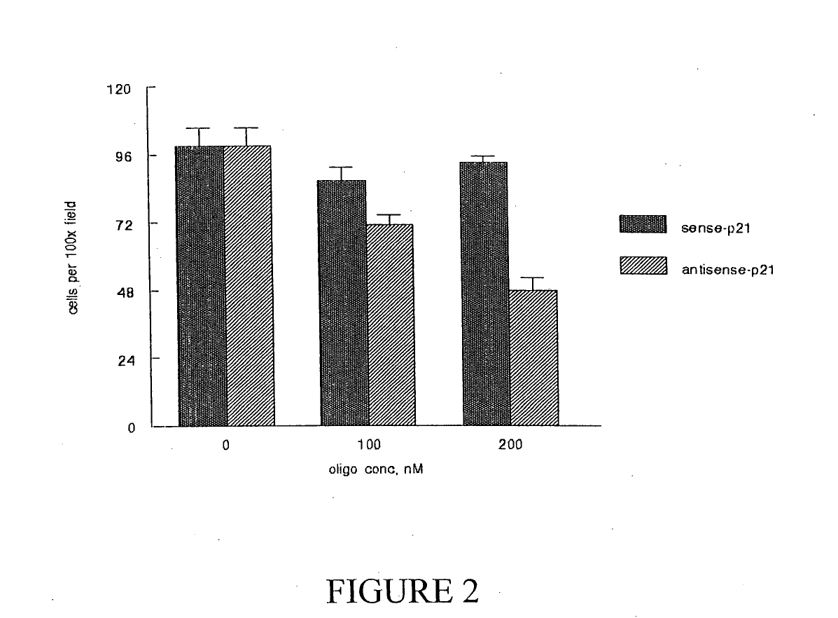

Antisense techniques were employed to examine the dependence of G1-phase progression on p21^{Waf1/Cip1} in the VSM cell lines (Crooke, 1993 Antisense research and applications. Boca Raton, CRC.). The oligodeoxynucleotides used were generated around the ATG start codon using GenBank sequences and were screened for lack of stable secondary structures or stable homodimer formation (OligoTech software, Oligonucleotides Etc., Wilsonville, Oreg.). Three independent controls were used in these experiments: (i) “dummy” transfection with Lipofectin but no DNA, (ii) random sequence oligodeoxynucleotide (SEQ. ID NO.:3) transfection, and (iii) sense p21^{Waf1/Cip1} oligodeoxynucleotide (SEQ. ID NO.:2) transfection. VSM cells were transfected with the appropriate oligodeoxynucleotide or control overnight in serum-free medium, and the next day the cells were stimulated with PDGF-BB (30 ng/ml) for another 18 hours. DNA synthesis was assessed by [³H]-thymidine incorporation and is expressed as mean ± s.e.m. of three wells per data point. The absolute counts differ between experiments due to different confluency of the cells. Significant inhibition of PDGF-stimulated DNA synthesis occurred when the cells were transfected with antisense p21^{Waf1/Cip1} (SEQ. ID NO.: 1), but not with sense p21^{Waf1/Cip1} (SEQ. ID NO.:2), “dummy” transfection (FIG. 1a), or random sequence (SEQ. ID NO.:3) control (FIG. 1b). To confirm that the observed growth inhibition was specific to the antisense p21^{Waf1/Cip1} oligodeoxynucleotide (SEQ. ID NO.:1), dose/response analysis were performed. There was inhibition of DNA synthesis with increasing concentration of antisense p21^{Waf1/Cip1} oligodeoxynucleotide (SEQ. ID NO.:1) up to 200 nM, with no effect of sense p21^{Waf1/Cip1} oligodeoxynucleotide (SEQ. ID NO.:2) (FIG. 1c). To demonstrate that this effect was not specific to the A10 cell line, a similar effect in a bovine VSM cell line (FIG. 1d). A10 VSM cells were transfected as in FIG. 1c. After 18 hours of PDGF incubation, the cells were counted by examining representative fields at 100× magnification. The average number of cells in 4 random fields in each well was determined. Changes in cell number were shown to parallel the alterations in DNA synthesis (FIG. 2).

2. Determination of Transfection Efficiency of the p21^{Waf1/Cip1} Antisense Oligonucleotide Agents.

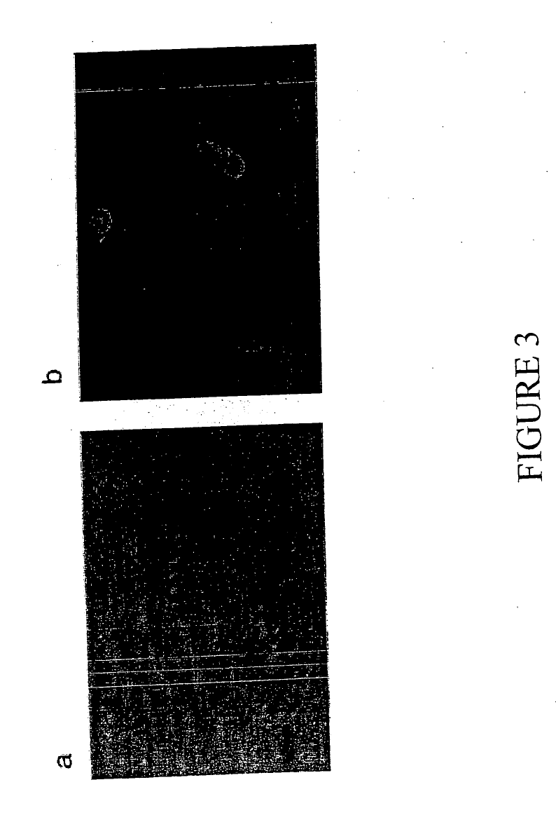

To establish whether the oligodeoxynucleotides crossed the cell membrane and entered the nucleus in order to inhibit p21^{Waf1/Cip1} protein production, cells were transfected with a fluorescein-tagged p21^{Waf1/Cip1} antisense oligodeoxynucleotide (with the same sequence as the p21^{Waf1/Cip1} antisense) and were examined for transfection efficiency. Upon examination by fluorescence microscopy, these cells demonstrated 100% transfection efficiency (FIG. 3), as has been reported for this technique (Coats et al., 1996 Science, 272, p. 877-880).

3. Determination of Effect of p21^{Waf1/Cip1} Antisense Oligonucleotides on p21^{Waf1/Cip1} Expression Levels.

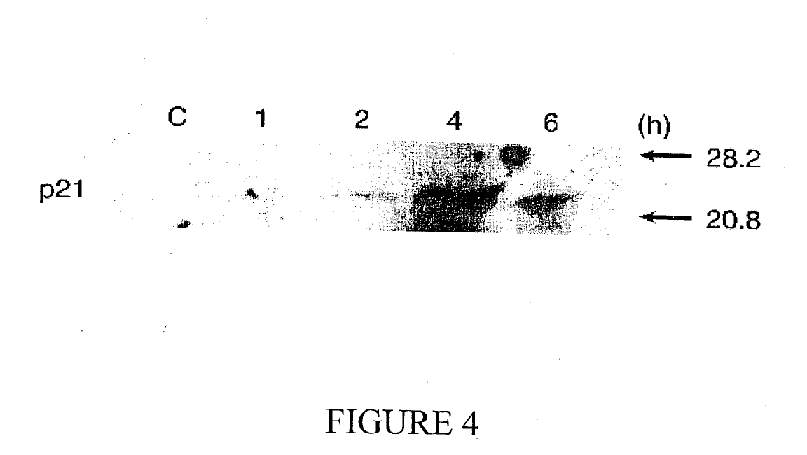

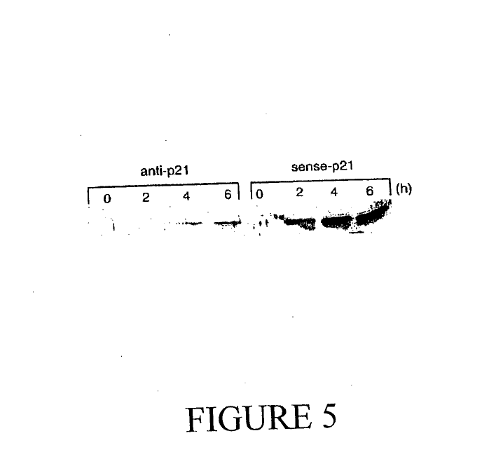

To determine whether antisense transfection with p21^{Waf1/Cip1} antisense oligonucleotides indeed decreases p21^{Waf1/Cip1} protein levels, p21^{Waf1/Cip1} levels after antisense transfection were examined employing the fact that PMA is a potent inducer of p21^{Waf1/Cip1} (Huang et al., 1995 Proc. Natl. Acad. Sci. U.S.A., 92, p. 4793-4797; Michieli et al., 1994 Cancer Res., 54, p. 3391-3395). Since p21^{Waf1/Cip1} protein levels were induced in VSM cells between 2 and 6 hours after PMA stimulation (FIG. 4), the p21^{Waf1/Cip1} levels in transfected cells after similar times of PMA stimulation were examined. After transfection with appropriate oligodeoxynucleotide and subsequent overnight incubation in quiescent media, antisense p21^{Waf1/Cip1} oligodeoxynucleotide (SEQ. ID NO.:1) caused significant attenuation of PMA-induced p21^{Waf1/Cip1} levels in VSM cells up to 6 hours (FIG. 5). There was no effect of the p21^{Waf1/Cip1} sense (SEQ. ID NO.:2) control oligodeoxynucleotide on cellular p21^{Waf1/Cip1} levels (compare with FIG. 4).

4. Determination of Specificity of p21^{Waf1/Cip1} Antisense Oligonucleotides (SEQ. ID NO.:1).

To check for specificity of protein inhibition by the antisense p21^{Waf1/Cip1} oligodeoxynucleotide (SEQ. ID NO.: 1), protein levels of p21^{Waf1/Cip1} and p27^{Kip1} after transfection with antisense p21^{Waf1/Cip1} oligodeoxynucleotide (SEQ. ID NO.:1) were examined. In these experiments the ability of antisense oligodeoxynucleotides (SEQ. ID NO.:1) to inhibit maximally stimulated CKI expression (see FIG. 4) was assessed. The cells were stimulated with PMA for 4 hours at various times after overnight serum starvation.

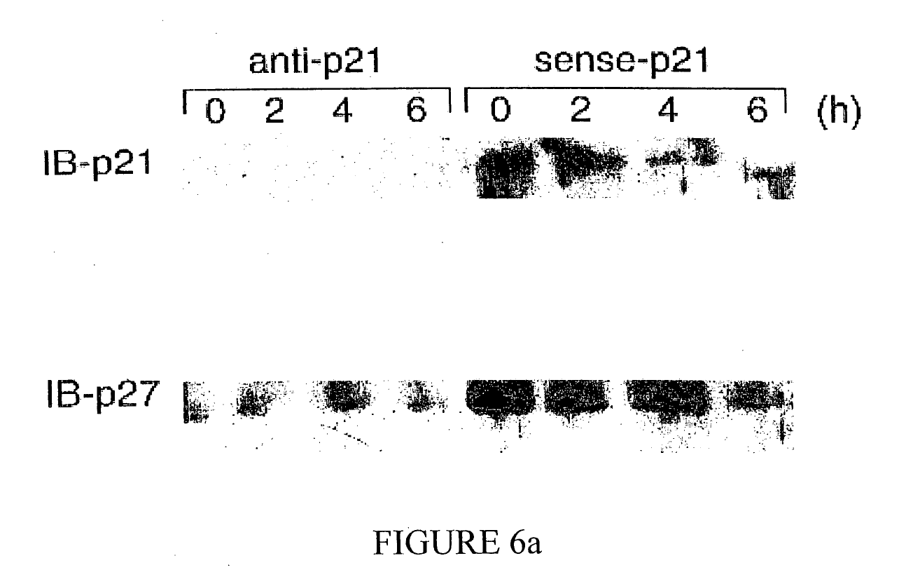

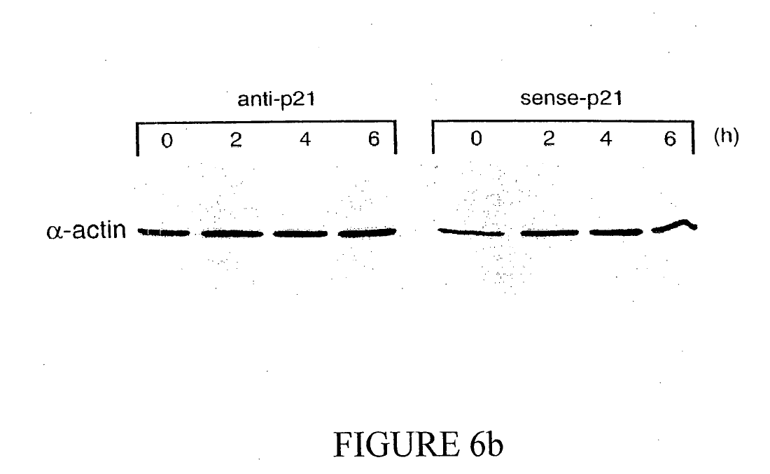

While antisense p21^{Waf1/Cip1} (SEQ. ID NO.:1) completely inhibited p21^{Waf1/Cip1} protein even after maximal stimulation with PMA, there was a slight decrease in p27^{Kip1} protein as well with this oligodeoxynucleotide (FIG. 6a). At the times indicated after the overnight incubation (in hours), the medium was changed to quiescence medium and the cells were stimulated with PMA for 4 hours to show maximal p21^{Waf1/Cip1} expression (FIG. 6a). The lysates were immunoblotted with (a) p21^{Waf1/Cip1} or p27^{Kip1} antibody or (b) α-actin antibody. This is likely due to sequence similarity between the two genes, as p21^{Waf1/Cip1} shares 43% sequence identity with p27^{Kip1} in the cdk/cyclin binding site (residues 27-88), located in the conserved N terminus (Nomura et al., 1997 Gene, 191, p. 211-218; Toyoshima and Hunter, 1994 Cell, 78, p. 67-74).

Levels of the VSM cell structural protein a-actin were not altered after transfection under identical conditions (FIG. 6b), demonstrating that the effect of antisense oligonucleotides (SEQ. ID NO.:1) on cell proteins was not a general inhibitory one. Furthermore, it is not believed that the slight p27^{Kip1} inhibition is playing a significant role in mitogenic inhibition, in light of data from other investigators (Rivard et al., 1996 J. Biol. Chem., 271, p. 18337-18341) based on the cyclin/cdk data discussed below.

5. Determination of the Effect of p21^{Waf1/Cip1} on the Association of Cyclin D1/cdk 4.

Cyclin D1/cdk 4 interaction was examined to determine this association as a possible mechanism of the permissive effect on growth of p21^{Waf1/Cip1} in VSM cells. Because other CKIs, such as p27^{Kip1}, have been shown to affect cyclin E/cdk 2 interaction (Cheng et al., 1998 Proc. Natl. Acad. Sci. USA., 95, p. 1091-1096; Polyak et al., 1994 Cell, 78, p. 59-66), the nature of this association was also examined. While the CKIs have been shown to be growth inhibitors in VSM cells (Chang et al., 1995 J. Clin. Invest., 96, p. 2260-2268; Fukui et al., 1997 Atherosclerosis, 132, p. 53-59; and Matsushita et al., 1998 Hypertension, 31, p. 493-498), various CKIs have been reported to act as “assembly factors” in other cells, both in vivo (Cheng et al., 1999 EMBO J., 18, p. 1571-1583) and in vitro (LaBaer et al., 1997 Genes Dev., 11, p. 847-862), yet previous studies have not shown inhibition of growth with interference of cyclin/cdk association. Since the cyclin D1/cdk 4 interaction occurs early after growth factor stimulation (reviewed in (Arellano and Moreno, 1997 Int. J. Biochem. Cell Biol., 29, p. 559-573)) and because this interaction is facilitated by p21^{Waf1/Cip1} and p27^{Kip1} in vivo (LaBaer et al., 1997: Genes Dev., 11, p. 847-862).

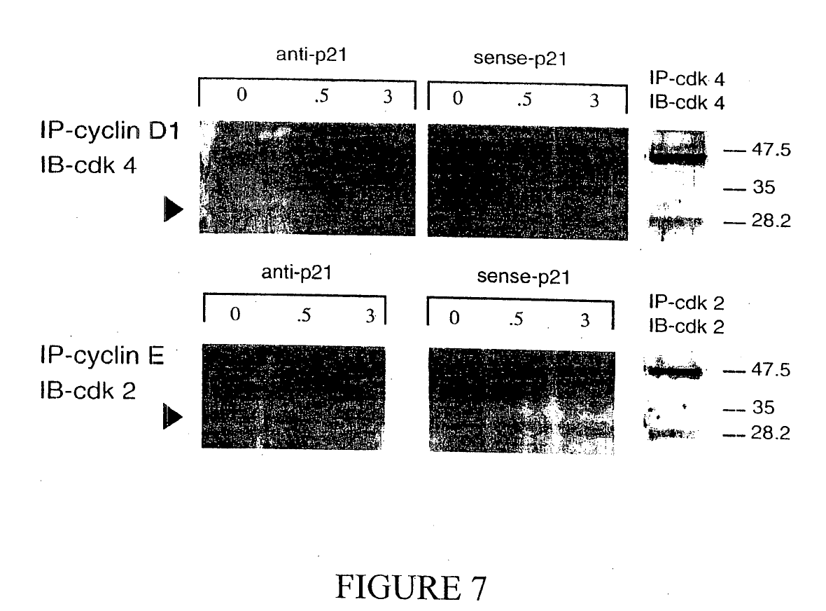

Cells were transfected with p21^{Waf1/Cip1} antisense (SEQ. ID NO.:1) or sense oligodeoxynucleotide (SEQ. ID NO.:2), allowed to grow overnight in serum-free media. After overnight incubation in serum-free medium, the cells were stimulated with PDGF-BB (30 ng/ml) for the times indicated. The cells were subsequently immunoprecipitated with either cyclin D1 or cyclin E and immunoblotted with cdk 4 or cdk 2, respectively. Antisense p21^{Waf1/Cip1}-transfected cells showed a marked decrease in association of cyclin D1 and cdk 4 at all times of PDGF stimulation, with no change in the cyclin E/cdk 2 interaction (FIG. 7). Thus the inhibitory effect of antisense p21^{Waf1/Cip1} oligodeoxynucleotide (SEQ. ID NO.:1) in VSM cells is likely by means of disruption in cyclin D1/cdk 4 interaction and thus prevention of activation of cdk 4 by cyclin D1.

Abnormal proliferation of VSM-like cells is pathogenic for a variety of diseases, such as atherosclerosis and angioplasty restenosis (Ross, 1993 Nature, 362, p. 801-809), as well as renal mesangial cell proliferation (Megyesi et al., 1999 Proc. Natl. Acad. Sci. US.A., 96, p. 10830-10835), thus the mechanism by which these cells are stimulated to grow is important in designing antiproliferative therapies for treating these and other diseases. Published studies in VSM cells focus on the antiproliferative action of CKI overexpression (Chang et al., 1995 J. Clin. Invest., 96, p. 2260-2268; Fukui et al., 1997 Atherosclerosis, 132, p. 53-59; Matsushita et al., 1998 Hypertension, 31, p. 493-498; and Smith et al., 1997: Genes Dev., 11, p. 1674-1689), and there are even some studies promoting the idea that pharmacological methods to increase p21^{Waf1/Cip1} may be useful in preventing the VSM cell proliferation seen after coronary angioplasty (Kusama et al., 1999 Atherosclerosis, 143, p. 307-313; Takahashi et al., 1999 Circ.Res., 84, p. 543-550; and Yang et al., 1996 Semin. Interv. Cardiol., 1, p. 181-184).

The above results show for the first time that inhibition of p21^{Waf1/Cip1} efficiently blocks mitogen stimulated VSM cell proliferation.

The difference between data presented herein, as compared to that in the mouse cells, may well be due to cell type, but the finding of growth inhibition in cells lacking active p21^{Waf1/Cip1}, explains the “essential activator” role of p21 promulgated by that group (Cheng et al., 1999 EMBO J., 18, p. 1571-1583). Furthermore, since p21(−/−) mice appear to develop normally (Deng et al., 1995: Cell, 82, p. 675-684), it is conceivable that p21^{Waf1/Cip1} disruption only affects “adult” cells, or that redundant pathways for cell growth are not present in A10 cells. Nevertheless, the data herein is the first demonstrating that the presence of these pleiotropic molecules is required for growth-factor mediated G1 progression in any cell type.

While the antisense p21^{Waf1/Cip1} oligodeoxynucleotide (SEQ. ID NO.:1) clearly inhibits p21, there is also slight inhibition of p27^{Kip1} protein as well by this oligodeoxynucleotide (FIG. 3). This occurrence is likely due to the sequence similarity between the two genes (Nomura et al., 1997 Gene, 191, p. 211-218; and Toyoshima and Hunter, 1994 Cell, 78, p. 67-74). However, it is not believed that the slight amount of inhibition of p27^{Kip1} is actively inhibiting VSM cell growth in the experiments, since cyclinE/cdk2 association is not affected (Coats et al., 1996 Science, 272, p. 877-880; Polyak et al., 1994 Cell, 78, p. 59-66; and Ravitz et al., 1995 Cancer Res., 55, p. 1413-1416) (see FIG. 7). Furthermore, others have reported suppression of quiescence, not of G1-phase progression, in fibroblasts transfected with antisense p27^{Kip1} (Rivard et al., 1996: J. Biol. Chem., 271, p. 18337-18341). In any case, the finding of unchanged α-actin protein levels in antisense p21 transfected cells argues against a general suppressive effect of these oligodeoxynucleotides (or of the process of transfection) on protein transcription.

Very recent work has shown that lack of a finctional p21^{Waf1/Cip1} gene in transgenic mice ameliorates progression of chronic renal failure after partial renal ablation (Megyesi et al., 1999 Proc. Natl. Acad. Sci. U.S.A., 96, p. 10830-10835). PCNA was found to be significantly increased in p21 (−/−) animals, but the degree of mesangial expansion was not quantitated. While the decrease in progression of renal failure was assumed to be due to a more hyperplastic (rather than hypertrophic) reaction in the p21(−/−) animals, the data disclosed in this example may shed some light on this phenomenon by suggesting that the response in p21(−/−) animals may have been a result of decreased mesangial cell mitogenesis due to inhibition of p21^{Waf1/Cip1} expression. This point of view is reinforced by others, noting that “all kidney growth parameters reported by Megyesi et al (Megyesi et al., 1999 Proc. Natl. Acad. Sci. U.S.A., 96, p. 10830-10835) are lower in p21(−/−) mice compared to p21(+/+) mice” (Al-Awqati and Preisig, 1999 Proc. Natl. Acad. Sci.U. S.A., 96, p. 10551-10553).

Thus, these results support use of antisense p21^{Waf1/Cip1} oligonucleotides (SEQ. ID NO.:1) to treat diseases involving abnormal VSM (or similar type) cell proliferation, such as atherosclerosis, angioplasty re-stenosis, and renal disease. While the available research on the CKIs in VSM cells has focused on the induction of the Cip/Kip family of CKIs in the presence of antiproliferative situations (Chen and Gardner, 1998 J. Clin. Invest., 102, p. 653-662; Fukui et al., 1997 Atherosclerosis, 132, p. 53-59; Kusama et al., 1999 Atherosclerosis, 143, p. 307-313; and Perlman et al., 1998 J. Biol. Chem., 273, p. 13713-13718), the inhibitory effects of CKI antisense constructs that were shown here may well be specific to vascular-like cells, as they were not observed in A431 cells (Example II, infra and reference (Ohtsubo et al., 1998 Oncogene, 16, p. 797-802).

### EXAMPLE II

### The Permissive Effect of p21^{Waf1/Cip1} on DNA Synthesis in p53-Inactive Cells

Materials: Human recombinant PDGF-BB was obtained from UBI (Lake Placid, N.Y.). Mouse monoclonal p21^{Waf1/Cip1} and cyclinD1, goat polyclonal cdk2 and cdk4, and rabbit polyclonal cycline antibodies were obtained from Santa Cruz Biotechnology (Santa Cruz, Calif.). Anti-goat horseradish peroxidase-conjugated IgG was obtained from Santa Cruz Biotechnology (Santa Cruz, Calif.). Lipofectin® was obtained form Life Technologies (Rockville, Md). Reagents for the Enhanced Chemiluminescence system and [³H]thymidine were obtained from Amersham (Arlington Heights, Ill.). All other reagents, including mouse monoclonal a-actin antibody, were from Sigma (St. Louis, Mo.).

Cell culture and DNA synthesis: Cultures of A10 and A431 cells were obtained from American Type Culture Collection (Rockville Md), were maintained as described (Weiss et al. 1998, Am. J. Physiol. 274, C1521-C1529), and were used between passages 15 and 24 or 25 and 35, respectively. The cells were growth-arrested by placing them in serum-free quiescence medium, exposed to growth factors as indicated, and [³H]thymidine incorporation assessed as previously described (Weiss and Nuccitelli, 1992, J. Biol. Chem. 267, 5608-5613).

Antisense transfections: Phosphorothioate antisense oligodeoxynucleotides were synthesized by Oligonucleotides Etc. (Wilsonville, Oreg.). The p21^{Waf1/Cip1} antisense vector was designed around the start codon of rat p21^{Waf1/Cip1}, with sequence 5′-GAC ATC ACC AGG ATC GGA CAT-3′ (SEQ. ID NO.:1). The sense p21^{Waf1/Cip1} sequence is 5′-ATG TCC GAT CCT GGT GAT GTC-3′ (SEQ. ID NO.:2). The scrambled random sequence control oligodeoxynucleotide was 5′-TGG ATC CGA CAT GTC AGA-3′ (SEQ. ID NO.:3). For the lipofection procedure, cells were grown to 90% confluence, the appropriate concentration of oligodeoxynucleotide was mixed with 6.6 μL of Lipofectin® per ml of Opti-MEM medium and was added to the cells for 4 hours at 37° C. Serum-free medium (without oligodeoxynucleotide) was added overnight, the media was changed in the morning and the cells were incubated in serum-free medium for the times indicated.

Western blots: Cells were grown to confluence in 6 cm culture dishes and serum deprived. After transfection and or treatment with appropriate agonist, the cells were washed with phosphate-buffered saline and lysed in lysis buffer and the supernatant was Western blotted as described (Weiss and Nuccitelli, 1992 J. Biol. Chem., 267, p. 5608-5613).

Example I shows that the CKI p21^{Waf1/Cip1}, while growth inhibitory in most situations, can also serve a permissive role in VSM cell growth. The mechanism behind this biphasic phenomenon is not yet known. In an attempt to further elucidate the nature of this effect, this study was designed to examine a cell line which is deficient in the immediate upstream regulator of p21^{Waf1/Cip1}. A431 cells, derived from a human squamous carcinoma, possess an inactive p53 protein (Kwok et al., 1994 Cancer Res., 54, p. 2834-2836) and are thus useful for assessing p53-independent effects of p21^{Waf1/Cip1}.

1. Determination of the Effect of p21^{Waf1/Cip1} in the A431 Cells.

In order to determine whether p21^{Waf1/Cip1} exerts a permissive effect on growth in cells which do not possess an active p53 protein, oligodeoxynucleotides encoding antisense (SEQ. ID NO.:1) and sense (SEQ. ID NO.:2) sequences which were generated around the ATG translational start codon were utilized as in Example I.

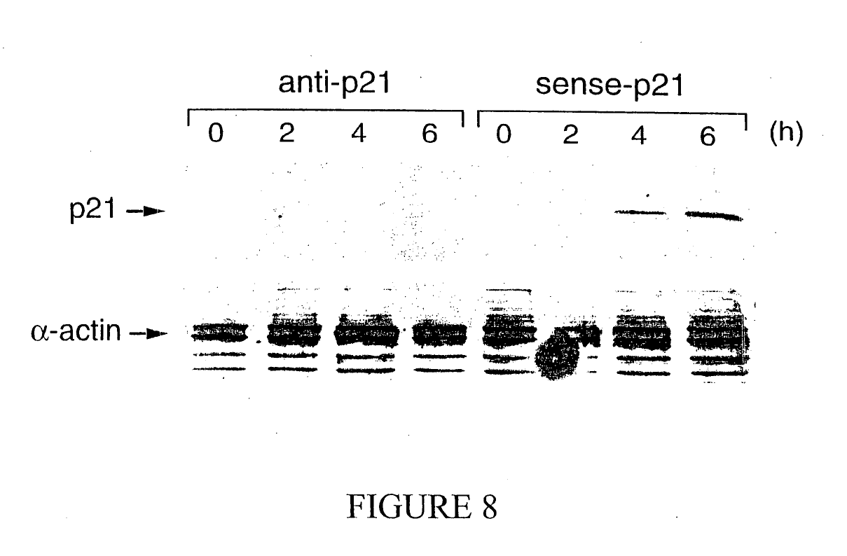

PMA is a potent inducer of p21^{Waf1/Cip1} in a variety of different cell lines (Huang et al., 1995 Proc. Natl. Acad. Sci. US.A., 92, p. 4793-4797; Michieli et al., 1994 Cancer Res., 54, p. 3391-3395) and was employed to examine p21^{Waf1/Cip1} levels after antisense transfection and confirm efficacy of translation to attenuate p21^{Waf1/Cip1} levels. A431 cells were transfected with 200 nM antisense p21^{Waf1/Cip1} (SEQ. ID NO.:1) or sense p21^{Waf1/Cip1} oligodeoxynucleotide (SEQ. ID NO.:2). After overnight incubation in serum-free medium, the cells were stimulated with PMA (100 ng/ml) for the times indicated, lysed, and the lysates were Western blotted with p21^{Waf1/Cip1} or α-actin antibody. Antisense p21^{Waf1/Cip1} oligodeoxynucleotide (SEQ. ID NO.:1) caused significant attenuation of PMA-induced p21^{Waf1/Cip1} levels in A431 cells up to 6 hours; there was no effect of the p21^{Waf1/Cip1} sense control oligodeoxynucleotide (SEQ. ID NO.:2) on cellular p21^{Waf1/Cip1} levels as compared to other p53-independent cell lines (Zeng and el-Deiry, 1996, Oncogene 12, 1557-1564) when stimulated with phorbol ester (FIG. 8). Levels of the structural protein α-actin were not altered after transfection under identical conditions (FIG. 8), demonstrating that the effect of antisense oligonucleotides (SEQ. ID NO.:1) on cell proteins was not a general inhibitory one towards protein translation.

2. Effect of p21^{Waf1/Cip1} as an “Assembly Factor” Role in Growth of A431 Cells

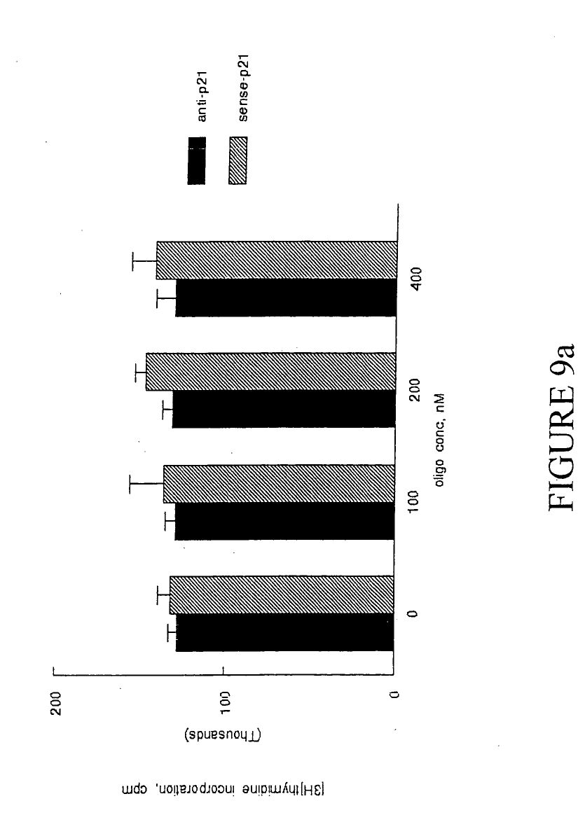

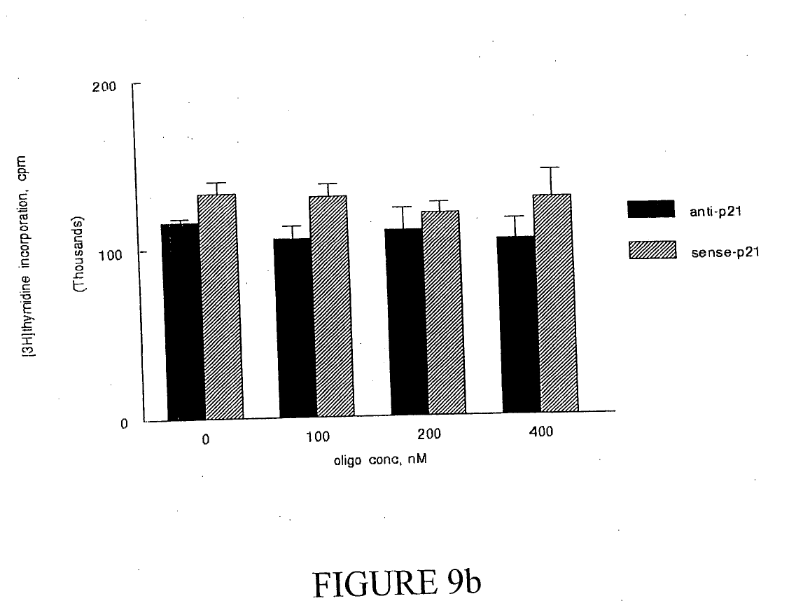

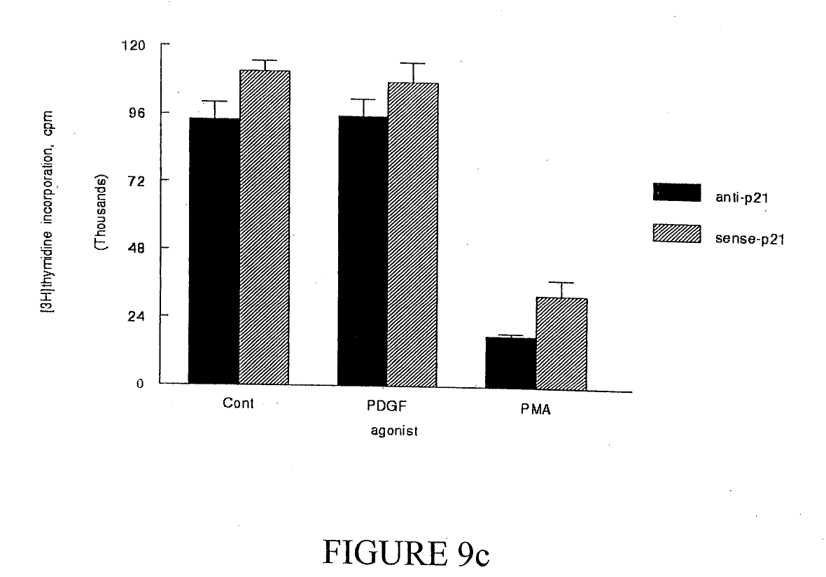

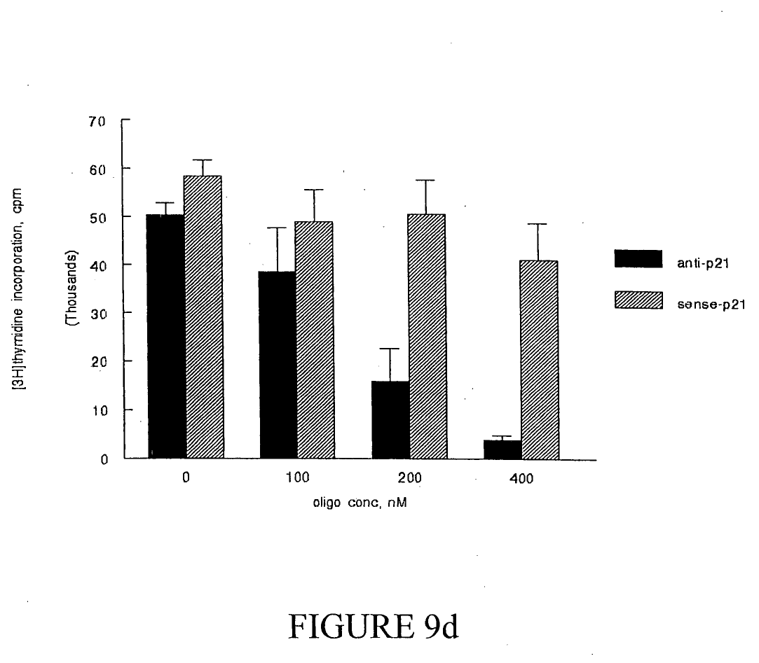

In order to determine whether p21^{Waf1/Cip1} serves an “assembly factor” role in growth of A431 cells as was observed in VSM cells of Example I, the DNA synthesis in these cells after transfection with antisense (SEQ. ID NO.:1) or sense p21^{Waf1/Cip1} (SEQ. ID NO.:2) oligodeoxynucleotides and subsequent stimulation with PDGF or serum was examined. There was no significant change in DNA synthesis upon stimulation of the cells with both of these growth factors (FIG. 9a, b), suggesting that p21^{Waf1/Cip1} does not serve an essential role in growth in A431 cells. Further experiments showed no difference in DNA synthesis between sense (SEQ. ID NO.:2) and antisense oligodeoxynucleotide (SEQ. ID NO.:1) up to 800 nM. As a control for the ability of the transformed A431 cell line to in fact be growth inhibited, these cells were incubated with PMA and showed significant growth inhibition with this agent (FIG. 9c) as has been demonstrated in variety of other cell lines (Weiss et al., 1991 J. Cell. Physiol., 149, p. 307-312; and Weiss and Yabes, 1996 Am. J. Physiol. (Cell Physiol.), 270, p. C619-C627). This A431 data is in contrast with that obtained in a variety of VSM cell lines, where cell growth was inhibited by similar oligodeoxynucleotide concentrations in cell stimulated with both PDGF-BB and serum (FIG. 9d).

3. Determination of Mechanism of A431 vs VSM Cells Affected by Antisense p21^{Waf1/Cip1} (SEQ. ID NO.:1).

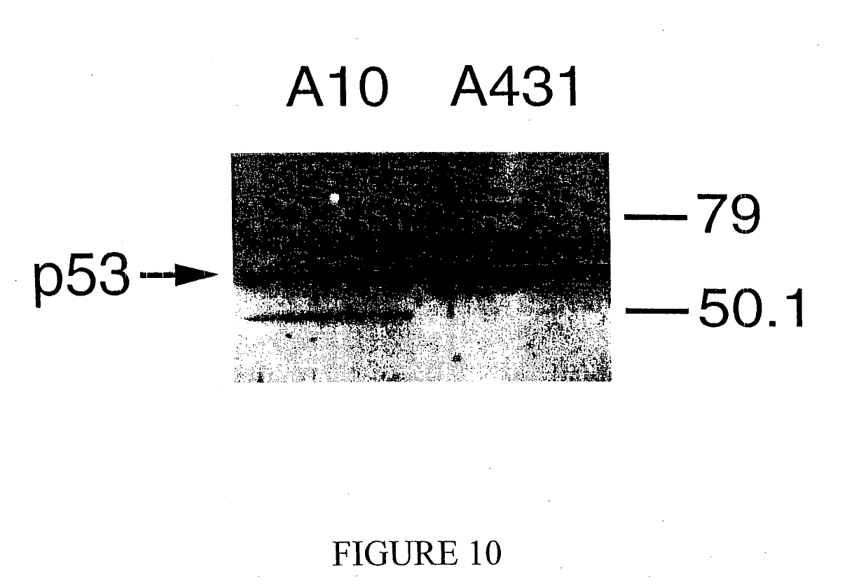

To begin to examine the mechanism by which A431 carcinoma cells are disparately affected by antisense p21^{Waf1/Cip1} (SEQ. ID NO.:1) as compared to VSM cells, the Rb status of these cells compared with A10 VSM cells was first examined. It is known that A431 cells possess a mutant p53 protein which renders this protein inactive (Kwok et al., 1994 Cancer Res., 54, p. 2834-2836). Lysates from non-serum-starved A431 and A10 cells were Western blotted with p53 antibody which recognizes both wild type and mutant forms of p53. DNA damaging agents are able to up-regulate the mutant form of this protein in these cells (Kwok et al., 1994 Cancer Res., 54, p. 2834-2836), yet, despite the differences in p53 activity in the two cell types, similar levels of this protein are found in both lines (FIG. 10).

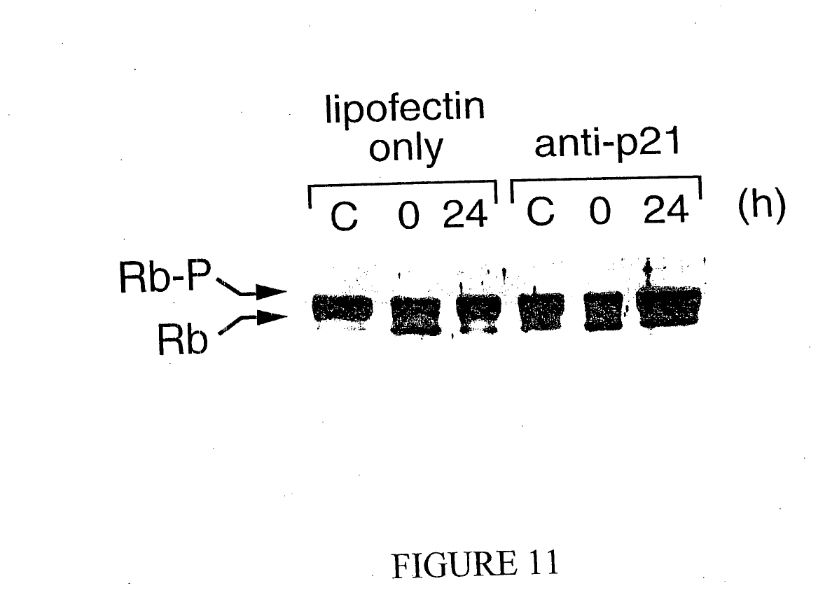

Upon activation by cyclin/cdk complexes, the Rb protein in turn becomes phosphorylated, causing it to release the transcription factors known as E2F, leading to early oncogene expression and ultimately to cell growth. An effect of p21^{Waf1/Cip1} on cdk/Rb interaction would be evident by a change in phosphorylation state of the Rb protein: this property can be assessed by examining small changes in gel mobility of this protein. A431 cells were transfected with 400 nM antisense p21^{Waf1/Cip1} oligodeoxynucleotide (SEQ. ID NO.:1) or with lipofectin only (no DNA). After overnight incubation in serum-free medium, the cells were stimulated with complete media for 6 or 24 hours, lysed, and the lysates were Western blotted with Rb antibody. Rb became hyperphosphorylated after 6 and 24 hours of 10% serum stimulation as evidenced by the appearance of a higher molecular weight band, and there was no difference between cells transfected (as control) with no DNA and those transfected with antisense p21^{Waf1/Cip1} oligodeoxynucleotide (SEQ. ID NO.:1) (FIG. 11), suggesting that p21^{Waf1/Cip1} has a minimal, if any, role in regulating this very distal part of the mitogenic signaling pathway in p53-inactive A431 cells.

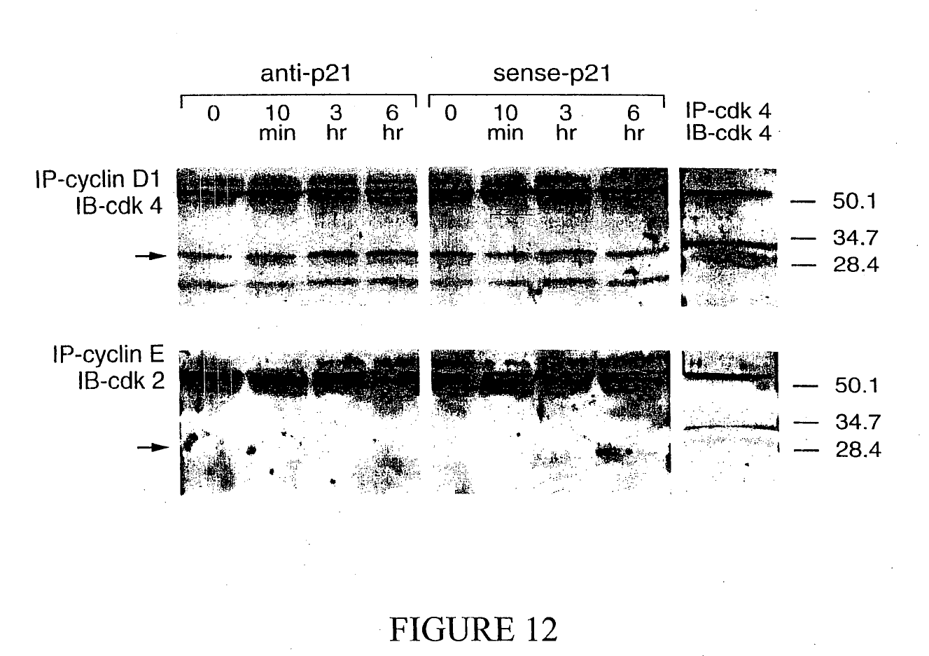

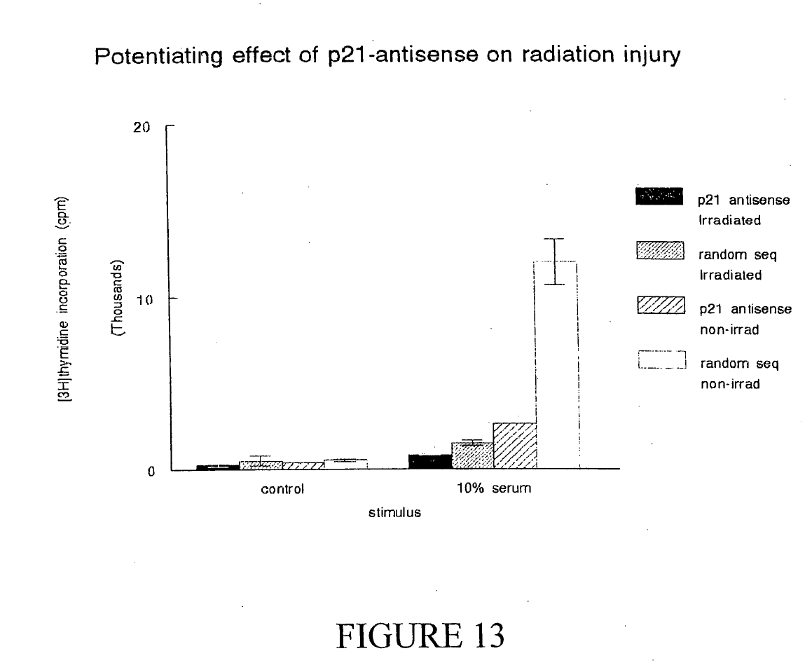

Cyclin/cdk interactions occur after growth factor stimulation and serve to integrate such responses with the CKIs and transmit them to the Rb/E2F systems, which leads to mitogenic signal transmission. The cyclin D1/cdk 4 interaction occurs early after growth factor stimulation (reviewed in (Arellano and Moreno, 1997 Int. J. Biochem. Cell Biol., 29, p. 559-573)), while the cyclinE/cdk2 interaction occurs late in G1 and is thought to have a role in triggering the actual onset of DNA replication after the cells have passed the restriction point (reviewed in (Sherr and Roberts, 1995: Genes Dev., 9, p. 1149-1163)). A431 cells were transfected with p21^{Waf1/Cip1} antisense (SEQ. ID NO.:1) or sense (SEQ. ID NO.:2) oligodeoxynucleotide, allowed to grow overnight in serum-free media, and then stimulated for various times with PDGF-BB. The cells were subsequently immunoprecipitated with either cyclinD1 or cyclinE and immunoblotted with cdk4 or cdk2, respectively. A lysate sample, showing the mobility of the immunoprecipitated cdks, confirmed the identity of the cdks. A431 cells were transfected with antisense (SEQ. ID NO.:1) or sense p21^{Waf1/Cip1} (SEQ. ID NO.:2) oligonucleotides. After overnight incubation in serum-free medium, the cells were stimulated with PDGF-BB (30 ng/ml) for the times indicated and immunoprecipitated (IP) with cyclin D1 or cyclin E and immunoblotted (IB) with cdk4 or cdk2. Unlike the situation in A10 VSM cells of Example I, there was no change in cyclinD/cdk4 association when p21^{Waf1/Cip1} expression was inhibited by antisense oligodeoxynucleotide (SEQ. ID NO.:1) (FIG. 12), suggesting that this protein is not required for association of these signaling proteins in these p53 inactive cells. Furthermore, there was no association of cyclinE with cdk2 at times ranging from 10 min to 6 hours of PDGF exposure, and there was no effect of p21^{Waf1/Cip1} inhibition in these experiments (FIG. 12).

As shown above in Example I, that the CKI p21^{Waf1/Cip1} can play a permissive role in growth of VSM cells, acting by allowing assembly of the cyclinD1/cdk4 complex, which in turn leads to events resulting in cell cycle transit. In Example II, the focus was on the direct upstream influence on p21^{Waf1/Cip1}: the tumor suppressor p53. To determine if p53 has any influence on the nature of the p21^{Waf1/Cip1} effect (stimulatory versus inhibitory on cell growth) has not been examined.

Example II shows that the permissive effect of p21^{Waf1/Cip1} on cell growth is not universal, as it does not occur in A431 cells stimulated either with PDGF-BB or serum. While the cell lines used, A431 and A10, are two distinct cell types, one a squamous carcinoma line and the other a smooth muscle line, the growth factor-stimulated mitogenic signaling pathways are believed to be quite similar, with the most obvious difference being that the A431 cells lack a functional p53 protein because of a mutation in the gene encoding this protein (Kwok et al., 1994 Cancer Res., 54, p. 2834-2836). So the cells that display permissive effect of p21^{Waf1/Cip1} on cell growth are the optimal targets of the present invention.

While the most likely explanation of this data is that a functional p53 protein is necessary for the permissive effect of p21^{Waf1/Cip1}, there exist several alternative scenarios. While it was observed that p21^{Waf1/Cip1} protein expression is specifically decreased by the antisense p21^{Waf1/Cip1} oligodeoxynucleotide (SEQ. ID NO.:1), it is conceivable that the magnitude of the decrement in A431 cells using antisense oligodeoxynucleotides was less than that seen with the VSM cells, or that A431 cells can signal to Rb with lower levels of p21^{Waf1/Cip1} protein.

While not wishing to be bound by any theory, since it has been well established (El-Deiry et al., 1993, Cell 75, 817-825) that p53 induces transcription of the p21^{Waf1/Cip1} gene (which would lead to an increase in p21^{Waf1/Cip1} protein levels), it is possible that there exist extremely low levels of p21^{Waf1/Cip1} in the absence of finctional p53, as would occur in A431 cells. This may, in turn, result in the activation of alternative pathways which the cell has evolved to allow growth and circumvent any requirement for p21^{Waf1/Cip1} in cell cycle transit. That there exist alternate pathways for p53-mediated cell cycle arrest independent of p21^{Waf1/Cip1} is clear, since fibroblasts homozygous null for p21^{Waf1/Cip1} are only partially defective in their response to DNA damage (Brugarolas et al., 1995 Nature, 377, p. 552-557; Deng et al., 1995 Cell, 82, p. 675-684). This possible mechanism is further supported by the finding that primary fibroblasts from p21^{Waf1/Cip1} and p27^{Kip1}-null mice did not show overtly abnormal cell cycles, despite the finding by those investigators that overall cyclinD-dependent kinase activity was reduced below the assay limit of detectability (Cheng et al., 1999 EMBO J., 18, p. 1571-1583). Still other studies have shown an increased growth rate of p21(−/−) as compared to wild type mouse embryonic fibroblasts (Deng et al., 1995 Cell, 82, p. 675-684), and no apparent G1 block in human colorectal cancer cells (Waldman et al., 1995 Cancer Res., 55, p. 5187-5190). This is the first demonstration of the lack of a permissive role of p21^{Waf1/Cip1} in cells deficient in active p53.

Cells lacking p53 fail to arrest in response to a wide variety of DNA damaging agents. This has been shown to be due to the stabilization of the p53 protein and enhancement of its transcriptional activity leading to arrest at both G₂/M phases, possibly through transactivation of the 14-3-3 proteins (Hermeking et al., 1997 Mol. Cell, 1, p. 3-11), and at G₁/S, through up-regulation of p21^{Waf1/Cip1} (Brugarolas et al., 1995 Mol. Cell, 1, p. 3-11; and Deng et al., 1995: Cell, 82, p. 675-684). In light of this data, there may exist not only cross-talk between p53 and transcription of p21^{Waf1/Cip1}, but also an influence of the p53 protein on whether p21^{Waf1/Cip1} is growth inhibitory, or required for growth through an unknown mechanism.

In summary, the above Example II demonstrates that the permissive effect of the CKI p21^{Waf1/Cip1}, which was unequivocally demonstrated in A10 VSM cells, does not occur under similar conditions in A431 cells. Since the principle difference in the growth factor mitogenic signaling cascades between these two cell lines relates to a mutant and inactive p53 in the A431 cells, it is believed that the permissive effect of p21^{Waf1/Cip1} requires the presence of active p53 protein.

### EXAMPLE III

### Antisense p21^{Waf1/Cip1} Potentiates Ionizing Radiation- and Chemotherapy-Induced Cell Cycle Arrest in VSM Cells

Materials: PDGF-BB and mouse monoclonal anti-human p21^{Waf1/Cip1} were obtained from Upstate Biotechnology (Lake Placid, N.Y.). Rabbit polyclonal anti-human caspase-3 antibody and anti-goat horseradish peroxidase-conjugated IgG were obtained from Santa Cruz Biotechnology (Santa Cruz, Calif.). Lipofectin® was obtained from Life Technologies (Rockville, Md.). Reagents for the Enhanced Chemiluminescence system and [³H]thymidine were obtained from Amersham (Arlington Heights, Ill.). Adriamycin (doxorubicin) was obtained from Pharmacia & Upjohn (Kalamazoo, Mich.). All other reagents, including Hoechst 33258, were from Sigma Chemical Co. (St. Louis, Mo.).

Cell culture and DNA synthesis assays: Cultures of A10 aortic VSM and A431 sarcoma cells were obtained from American Type Culture Collection (Rockville Md.), and were maintained as described (Weiss R H, et al. Am J Physiol 274:C1521-C1529, 1998); the A10 cells were used between passages 15 and 25. Where indicated, the cells were growth-arrested by placing them in serum-free quiescence medium, exposed to PDGF-BB or 10% serum-containing medium as indicated, and [³H]thymidine incorporation was assessed as previously described (Weiss R H, Nuccitelli R: J Biol Chem 267:5608-5613, 1992).

Ionizing radiation experiments: Cells were transfected with oligodeoxynucleotide 16-24 hours prior to γ-irradiation. Cells were subjected to 1-12 Gy of γ-irradiation from a ¹³⁷Cs source. 30 minutes after irradiation cells were stimulated with PDGF-BB or 10% serum media. After 6 hours of stimulation, cells received 1 μCi [³H]thymidine per ml of media and were analyzed for DNA synthesis. For Western blots, cells were lysed 4 hours after irradiation unless stated otherwise.

Antisense transfections: Phosphorothioate antisense and random sequence control oligodeoxynucleotides were synthesized by Oligodeoxynucleotides Etc. (Wilsonville, Oreg.). The p21^{Waf1/Cip1} antisense vector was designed around the start codon of rat p21^{Waf1/Cip1}, with sequence 5′-GAC ATC ACC AGG ATC GGA CAT-3′ (SEQ. ID NO.:1). The scrambled random sequence control oligodeoxynucleotide was 5′-TGG ATC CGA CAT GTC AGA-3′ (SEQ. ID NO.:3). For the lipofection procedure, cells were grown to 60% confluence, washed with sterile phosphate-buffered saline, and the appropriate concentration of oligodeoxynucleotide was mixed with 6.6 μL of Lipofectin® per ml of Opti-MEM medium and was added to the cells for 4 hours at 37° C. Serum-free medium (without oligodeoxynucleotide) was added overnight, the media was changed in the morning and the cells were stimulated as indicated.

Western blots: Cells were grown to confluence in 6 cm culture dishes and serum deprived. After transfection and or treatment with appropriate agonist, conditioned medium was removed and saved and the cells were washed with phosphate-buffered saline and lysed in lysis buffer. Both supernatant and cell lysate were normalized to the lysate protein concentrations and Western blotted as described (Weiss R H, et al. Am J Physiol 274:C1521-C1529, 1998).

Apoptosis assays: Cells were grown on collagen-coated coverslips, and transfected as stated above. 16-24 hours after transfection, cells were exposed to 12 Gy dose of γ-irradiation or 0.5 μM wortmannin. 24 hours later, cells were fixed in 3.7% formaldehyde (diluted in PBS) for 10 minutes. The cells were rinsed with cold PBS and permeabilized using 0.1% Triton X-100 (diluted in deionized water) for 5 minutes. Rinsed again with PBS, the cells on the coverslips were submerged in Hoechst Staining solution (3.0 μl in 37.5 ml deionized water) for 5 minutes. Cells were given a final three rinses with cold PBS before being mounted in polyvinyl alcohol mounting medium. Cell nuclei were visualized using a ZEISS WL Microscope and photographed under 40×.

1. Effect of Ionizing Radiation and Adriamycin on Growth of VSM Cells

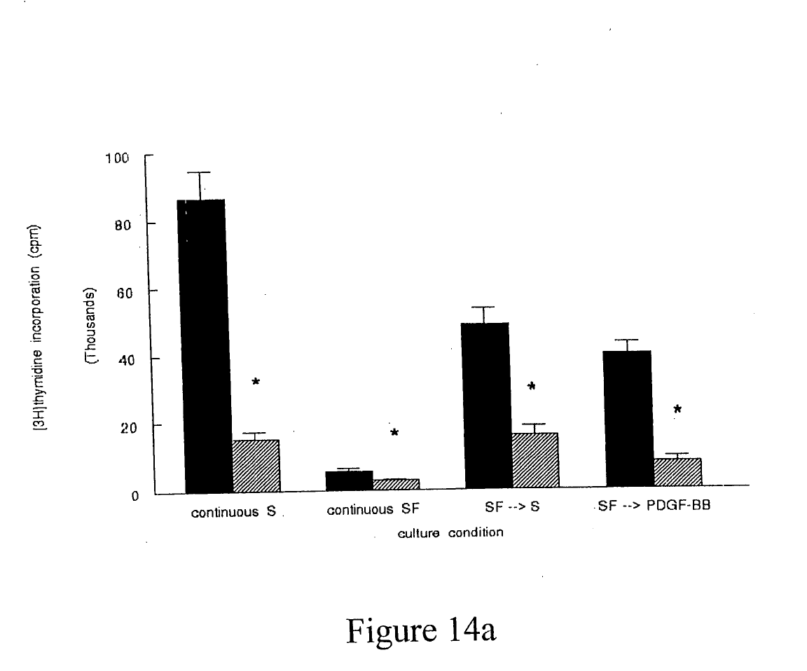

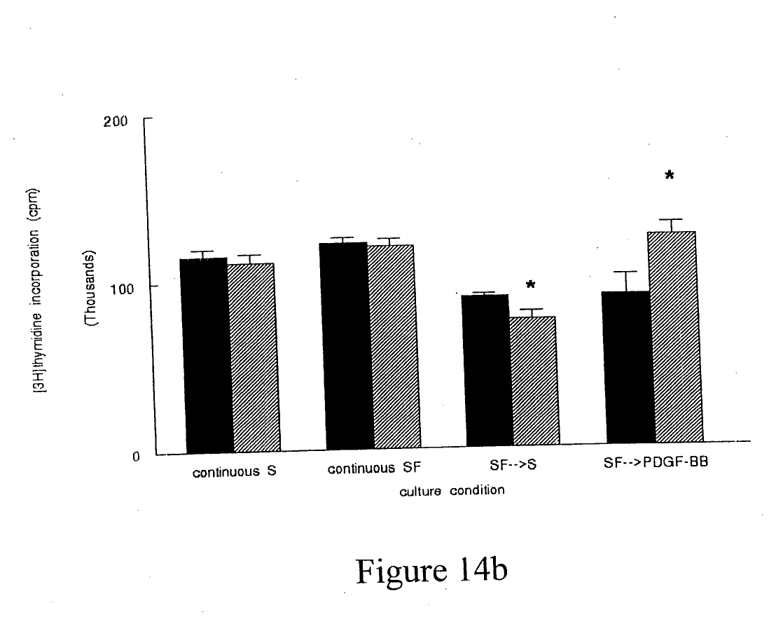

Both ionizing radiation and Adriamycin cause growth arrest due to induction of p53 as a result of DNA damage. This tumor suppressor protein sets in motion a series of events culminating in cell cycle arrest in G1 and G2 (Bunz F, et al. Science 282:1497-1501, 1998; Agarwal M L, et al. Proc Natl Acad Sci US A 92:8493-8497, 1995; and Poon R C, et al. J Biol Chem 271:13283-13291, 1996). Examples I and II utilized two established mesenchymal-derived cell lines, one (A10 VSM cells) possessing intact p53 and the other (A431 squamous carcinoma cells) which has a mutant p53 gene and an inactive p53 protein (Kwok T T, et al. Cancer Res 54:2834-2836, 1994). In order to characterize the response of these particular cells to DNA damage, growth of these cells in various stages of the cell cycle was examined. Upon removal of serum, “normal” cells generally remained in G0, and subsequent stimulation with serum or growth factors caused them to resume transit through the cell cycle. To set the stage for further studies, the cells were examined under four conditions: (1) when left in serum-containing medium; (2) when left in serum-free medium; (3) when stimulated with complete medium after serum-free medium, and (4) when stimulated with PDGF-BB after serum-free medium.

[³H]thymidine incorporation was examined, which is a measure of transit through S phase of the cell cycle, after the cells were exposed to irradiation. A10 VSM cells were growth arrested after exposure to ionizing radiation under all conditions examined, likely through p53-mediated induction of p21 and subsequent cdk inhibition (FIG. 14A). In A431 cells, which possess a mutant and inactive p53, ionizing radiation failed to cause consistent inhibition of DNA synthesis, although there was slight (but significant) inhibition in cells which were exposed to serum after serum starvation (FIG. 14B). This suggested that p53, and its downstream effector p21, are important in mediating the cell cycle arrest and DNA damage repair in cells exposed to ionizing radiation. Subsequent experiments in VSM cells were therefore performed in cells that were left in complete medium, since these were the conditions under which the changes with radiation were maximal, and because these conditions most closely replicate the in vivo milieu.

The above findings (FIGS. 14A and B) suggest that p53 status is essential to determining the response of cells to ionizing radiation and, by extension, to DNA damage. This is consistent with data reported by other investigators, as the level of p53 has been shown to be a very sensitive indicator of DNA damage. It has been suggested that one double-stranded DNA break is sufficient to induce this protein (Di Leonardo A, et al. Genes Dev 8:2540-2551, 1994). Furthermore, both ionizing radiation and Adriamycin are known to cause DNA damage, and have been shown to increase p53 expression in order to mediate G1 and G2 arrest, such that DNA repair can occur (Bunz F, et al. Science 282:1497-1501, 1998; and Agarwal M L, et al. Proc Natl Acad Sci USA 92:8493-8497, 1995). The downstream effector of p53 is p21, and since p21 has been shown to have variable effects on cell cycle events (Sherr C J, Roberts J M. Genes and Dev 13:1501-1512, 1999) and on growth (Weiss R H, et al. J Biol Chem 275:10285-10290, 2000; Weiss, R. H. and Randour, C. Cellular Signalling, 12:413-418, 2000), whether p21 attenuation affects cell growth in response to DNA damage, was studied.

2. Effect of p21^{Waf1/Cip1} Antisense Oligonucleotide (SEQ. ID NO.:1) on Cell Growth in Response to DNA Damage

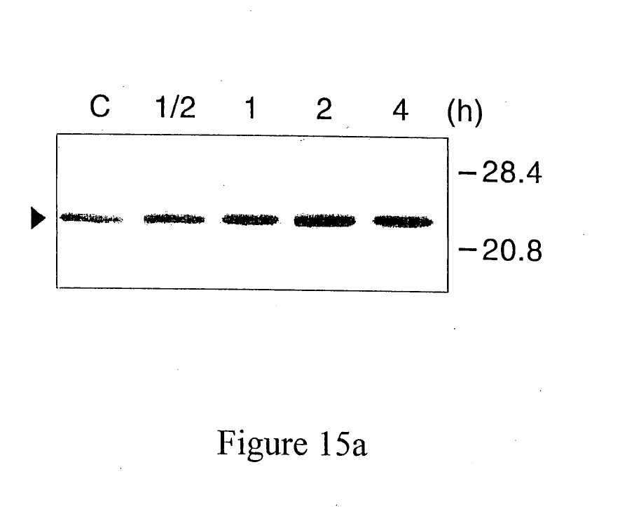

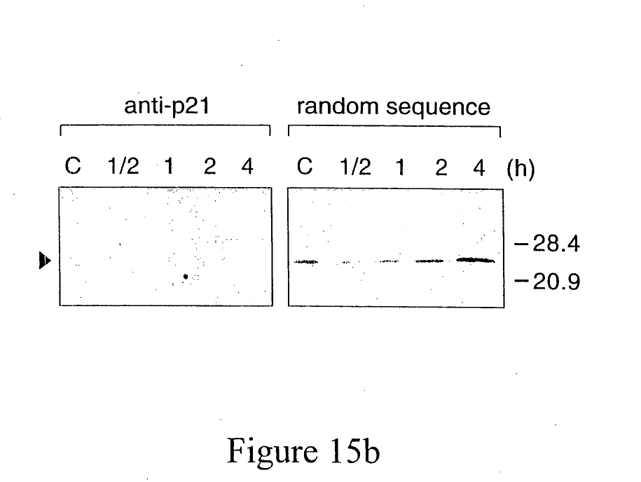

To confirm that p21 levels were increased after exposure to ionizing radiation, and that the oligonucleotides inhibit this phenomenon in A10 cells, p21 protein levels under these conditions were assayed. The level of p21 was increased after ionizing radiation (12 Gy) up to 4 hours (FIG. 15A). p21 levels were consistently low in all cells transfected with the antisense oligonucleotide to p21 and then stimulated with ionizing radiation (12 Gy). No peak of induction was observed in these cells (FIG. 15B). In the cells transfected with the random sequence control oligonucleotide, there was initial slight inhibition of p21 levels at ½ and 1 hours after ionizing radiation, but the levels rapidly increased by 4 hours (FIG. 15b). With the exception of the initial decrease in p21 levels at ½ and 1 hours, the results with the control oligonucleotides were similar to that observed in non-transfected cells. The specificity of the antisense oligonucleotide was previously demonstrated by showing no change in expression of α-actin in this cell line after antisense p21 oligonucleotide transfection (Weiss R H, et al. J Biol Chem 275:10285-10290, 2000; Weiss, R. H. and Randour, C. Cellular Signalling, 12:413-418, 2000).

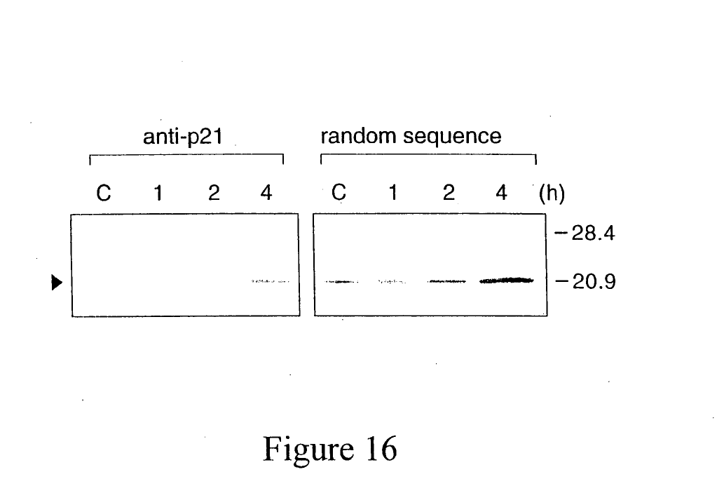

Adriamycin is a prototypical DNA damaging agent used in the treatment of a variety of cancers. The predominantly G2 arrest seen after Adriamycin treatment has been associated with an increase in p21 levels in some cell lines (Siu W Y, et al. FEBS Lett 461:299-305, 1999). In order to determine whether similar augmentation of p21 is seen in A10 VSM cells and whether the antisense p21 oligonucleotide is inhibiting this response, these cells were treated with Adriamycin either with or without first transfecting the cells with the antisense and control oligonucleotides. Confluent VSM cells were transfected with antisense oligonucleotide to p21 or random sequence control oligonucleotide as described above. After 24 hours, the cells were exposed to Adriamycin (500 ng/ml), lysed at the indicated times after exposure, and Western blotted with p21 antibody. As in the case of cells exposed to ionizing radiation, p21 levels were increased by Adriamycin at similar times, with a marked attenuation of this response in cells transfected with the antisense p21 oligonucleotide (FIG. 16).

3. Effect of the Levels of p21 on Growth Activity

Having successfully inhibited the peak of p21 expression in VSM cells after ionizing radiation as well as Adriamycin exposure, it was determined whether growth modulation, after these maneuvers, is altered after suppression of the peak p21 level, which occurs in normal cells when so stimulated.

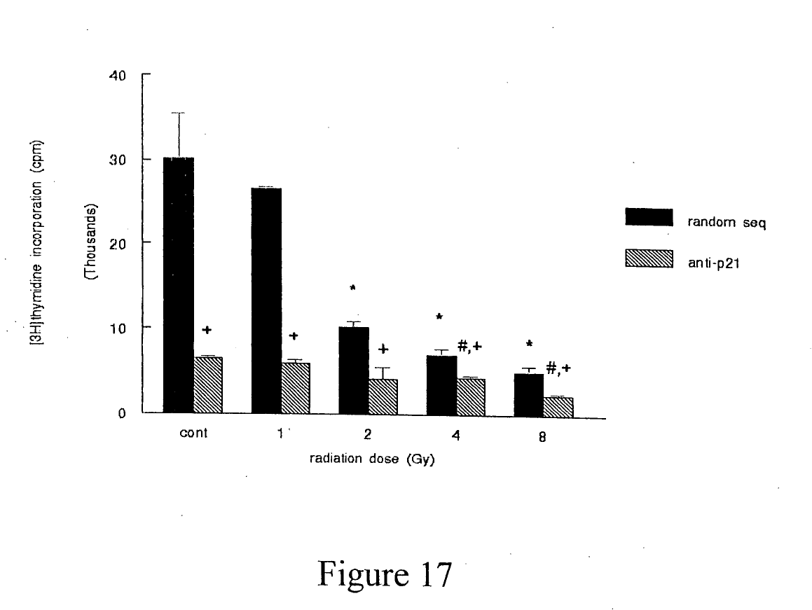

Confluent VSM cells were transfected with the antisense oligonucleotide to p21 (SEQ. ID NO.:1) (or a random sequence control oligonucleotide (SEQ. ID NO.:3)) and exposed to various doses of ionizing radiation. After 24 hours, the cells were exposed to the indicated dose of radiation. Six hours later, [³H]thymidine was added overnight and DNA synthesis was assessed as in FIG. 14. Cells transfected with the control oligonucleotide (SEQ. ID NO.:3) showed attenuation of DNA synthesis as a function of radiation dose from 1 to 12 Gy (FIG. 17). After transfection of the cells with the antisense p21 oligonucleotide, there was a marked inhibition of DNA synthesis in non-irradiated cells, as previously shown in Example I (Weiss R H, et al. J Biol Chem 275:10285-10290, 2000). The effects of irradiation on cell cycle arrest were potentiated at higher doses of radiation, with a maximum potentiation at 8 Gy. The potentiation at 12 Gy did not reach statistical significance, probably due to cell mortality at that dose.

4. Effect of Antisense Oligonucleotide-Mediated Inhibition of p21 on Adriamycin Induced Cell Cycle Arrest

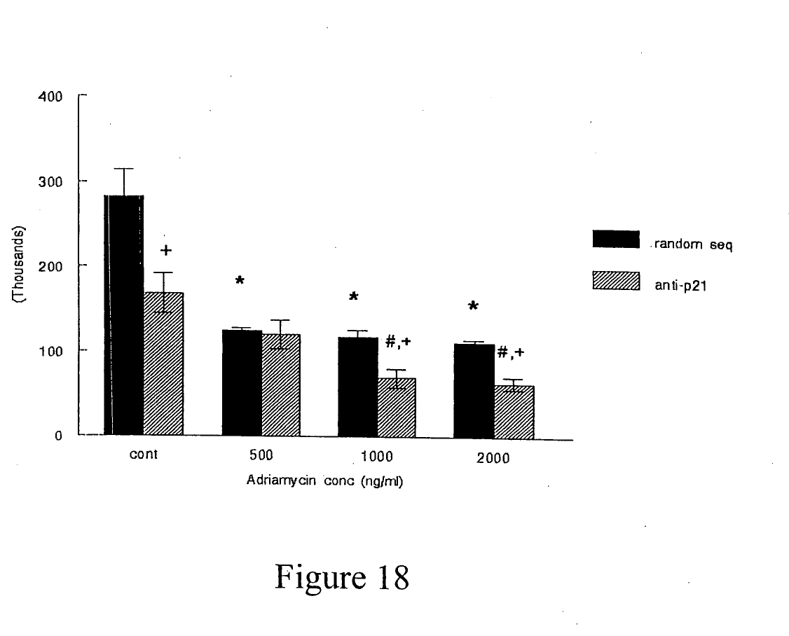

It has been previously shown that p21 (−/−) cells are more sensitive to the killing effects of a variety of chemotherapeutic agents (Waldman T, et al. Nature 381:713-716, 1996; Stewart Z A, et al. Cancer Res 59:3831-3837, 1999; Waldman T, et al. Nat Med 3:1034-1036, 1997; and Fan S et al. Oncogene 14:2127-2136, 1997). The effect of antisense oligonucleotide-mediated inhibition of p21 on Adriamycin induced cell cycle arrest was determined. Confluent VSM cells were transfected with control (SEQ. ID NO.:3) or antisense p21 oligonucleotides (SEQ. ID NO.:1) and exposed to Adriamycin at concentrations from 500 to 2000 ng/ml. After 24 hours, the cells were exposed for 2 hours to the indicated concentration of Adriamycin. Two hours later, [³H]thymidine was added overnight and DNA synthesis was assessed as in FIG. 14. While the growth inhibitory effect of Adriamycin on control oligonucleotide transfected cells plateaued in this range, there was significant potentiation of growth inhibition in antisense p21 oligonucleotide transfected cells when exposed to from 1000 to 2000 ng/ml Adriamycin (FIG. 18).

5. Effect of Antisense Oligonucleotide-Mediated Inhibition of p21 on Apoptosis

While it was shown that p21 is required for serum and PDGF-stimulated growth in VSM cells (Weiss RH, et al. J Biol Chem 275:10285-10290, 2000), there are also reports that the absence of p21 may cause cells to be converted from growth arrest to apoptosis (Tian H, et al. Cancer Res 2000 Feb 1;60 (3):679 -84 60:679-684, 2000).

In order to determine whether the potentiation of ionizing radiation and Adriamycin induced DNA synthesis inhibition by p21 antisense oligonucleotides is accompanied by apoptosis, caspase-3 activation in response to ionizing radiation was examined. Caspase-3 is an effector caspase whose activation (leading to apoptosis) results in a 20 kD cleavage product which can be assessed by Western blotting (McCarthy N J, Evan G I. Curr Top Dev Biol 36:259-278, 1998). This process is an early event in a cascade of reactions which ultimately leads to apoptosis, as is evident by the fact that inactivation of caspase-3 dramatically reduces apoptosis in several cell lines (Woo M, et al. Genes Dev 12:806-819, 1998).

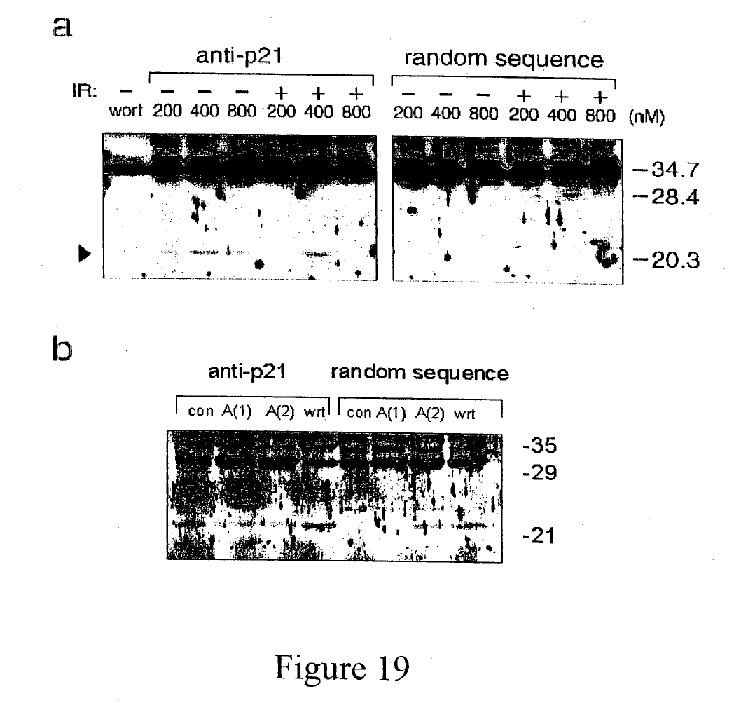

VSM cells were transfected with antisense or control oligonucleotides overnight and then stimulated with ionizing radiation. While ionizing radiation alone did not induce caspase-3 activation at 4 hours at radiation doses of 12 Gy, there was an impressive increase in caspase-3 activation in cells transfected with antisense p21 oligonucleotide, either alone or after exposure to ionizing radiation (FIG. 19A).

Adriamycin increases p53 and p21 resulting in cell cycle arrest (Siu W Y, et al. FEBS Lett 461:299-305, 1999). As in the case of ionizing radiation, caspase-3 activation was observed in cells transfected with antisense p21 (SEQ. ID NO.:1), but not random sequence oligonucleotides (SEQ. ID NO.:3), with little effect of Adriamycin alone on this apoptosis effector at the time examined (FIG. 19b).

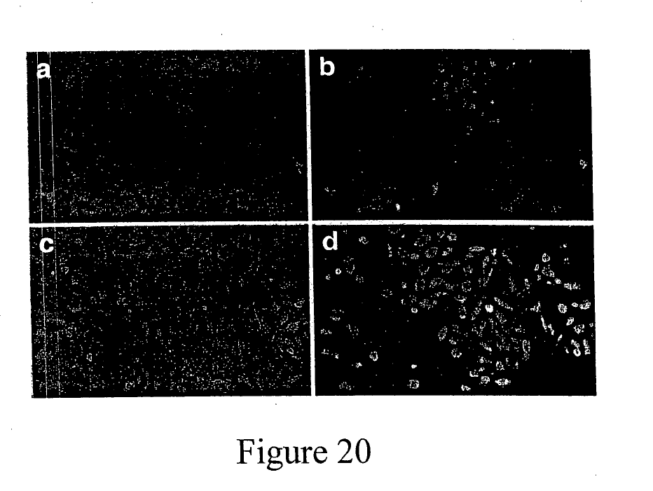

As another measure of apoptosis, transfected cells fixed and stained with Hoechst 33258 were examined. Cells transfected with antisense p21 (SEQ. ID NO.:1), but not random sequence control oligonucleotides (SEQ. ID NO.:3), showed extensive apoptotic changes 24 hours after transfection (FIG. 20).

The results described in this Example show apoptosis, as well as potentiation of cell cycle arrest in VSM cells using a simple and straightforward technique, suggesting a new paradigm for treatment of fibrotic diseases, such as angioplasty restenosis, hemodialysis graft stenosis, mesangial proliferative glomerular disease, as well as in most pathogenic models of atherosclerosis. Indeed, the findings that transfection of the antisense p21 oligonucleotide alone induced apoptosis, coupled with the findings from other investigators that p21 (−/−) mice do not have phenotypic changes or an increased susceptibility to spontaneous tumors (Deng C, et al. Cell 82:675-684, 1995), suggests the use of antisense p21 oligonucleotides as cancer therapeutics (see Tian H, et al. Cancer Res 2000 February 1;60 (3):679 -84 60:679-684, 2000).

While the CKIs had been considered to be solely growth inhibitory, data is emerging that these molecules in fact have both positive and negative effects on cell cycle checkpoints (Sherr C J, Roberts J M. Genes and Dev 13:1501-1512, 1999) as well as on cell growth (Weiss R H, et al. J Biol Chem 275:10285-10290, 2000). Furthermore, since p21 arrests transit through the cell cycle at G1 in order for DNA damage repair to occur (Bunz F, et al. Science 282:1497-1501, 1998), disruption of this checkpoint in p21(−/−) cells results in multiploidy and subsequent targeting of the cells for apoptosis (Waldman T, et al. Nature 381:713-716, 1996; and Mantel C, et al. Blood 93:1390-1398, 1999).

The above results demonstrate that using a novel antisense oligonucleotide corresponding to the translational start site of the p21 gene to attenuate p21 levels (Example I), arrest of the cell cycle and apoptosis occurs in VSM cells which have had p21 levels reduced using p21 inhibitory agents. Furthermore, under these conditions cell cycle inhibitory responses to radiation, and the DNA damaging chemotherapeutic agent, Adriamycin, are both potentiated.

While use of antisense oligonucleotides to p21 demonstrated a clear potentiation of apoptosis, after exposure of the cells to DNA damaging agents, it was surprising that caspase-3 activation did not occur with either radiation or Adriamycin alone at the times examined, despite inhibition of cell cycle transit as assessed by DNA synthesis. While not wishing to be bound by any particular theory, it is possible that caspase-3 was cleaved at a later time that was not apparent in the gels examined. In any case, the propensity of the antisense p21 oligonucleotide to initiate apoptosis and lead to its morphological characteristics in VSM cells is abundantly clear from these results.

The application of p21 inhibitors in renal disease, in addition to their role as vascular cell growth attenuators, has become evident in a recent study showing that p21(−/−) mice, as compared to wild type, were less likely to develop chronic renal failure after renal ablation (Megyesi J, et al. Proc Natl Acad Sci USA 96:10830-10835, 1999). While these investigators suggested that this effect was due to their finding that the absence of the p21 gene leads to a more hyperplastic response, apoptosis of mesangial or other renal cells may also be contributory. Other renal investigators have shown that diabetic p21 (−/−) mice do not develop the same degree of glomerular hypertrophy as their wild type counterparts (Al Douahji M, et al. Kidney Int 56:1691-1699, 1999), an effect which may, in light of the data herein, also be due to apoptosis of glomerular cells.

The likelihood that antisense oligonucleotides may have potential therapeutic utility is further bolstered by experiments in animal models. Data has been generated on the pharmacology of antisense oligonucleotides in animal models. For example, the acute LD₅₀ of phosphorothioates is 500 mg/ml (Crooke, S. T. Therapeutic applications of oligonucleotides. 1995. Austin, R. G. Landes), well above the effects seen herein at nanomolar quantities of p21 antisense oligonucleotides. Furthermore, phosphorothioate oligonucleotides are rapidly and extensively absorbed after intravenous administration in rats, and distribute broadly to all peripheral tissues, especially liver, kidney, bone marrow, skeletal muscle and skin (Crooke, S. T. Therapeutic applications of oligonucleotides. 1995. Austin, R. G. Landes). An antisense oligonucleotide to c-raf injected into nude mice implanted with human tumors showed decent tissue uptake without the benefit of lipofection reagents (Monia BP, et al. J Biol Chem 267:19954-19962, 1992. The data presented herein showing the effect of the novel antisense cyclin kinase inhibitors of the invention in vascular cells support the use of the compositions and methods of the invention in the treatment of vascular and renal proliferative diseases.

### EXAMPLE IV

### The Effect of p21^{Waf1/Cip1} on TBF-β-Mediated Matrix Protein Secretion

Materials: TGF-β1 and mouse monoclonal anti-human p21^{Waf1/Cip1} were obtained from Upstate Biotechnology (Lake Placid, N.Y.). Polyclonal anti-rat fibronectin and laminin antibodies were obtained from Chemicon (Temecula, Calif.). Anti-goat horseradish peroxidase-conjugated IgG was obtained from BioRad (Richmond, Calif.). Lipofectin® was obtained from Life Technologies (Rockville, Md.). Reagents for the Enhanced Chemiluminescence system and [³H]thymidine were obtained from Amersham (Arlington Heights, Ill.). All other reagents, including mouse monoclonal α-actin antibody and protein A-Sepharose beads, were from Sigma Chemical Co.(St. Louis, Mo.).

Cell culture, DNA synthesis, and proliferation assays: Cultures of A10 aortic VSM cells were obtained from American Type Culture Collection (Rockville Md.), were maintained as described (Weiss, R. H.; et al. Am. J. Physiol. 274: C1521-C1529; 1998), and were used between passages 15 and 25. The cells were growth-arrested by placing them in serum-free quiescence medium, exposed to TGF-β or 10% serum-containing medium as indicated in the figures, and [³H]thymidine incorporation was assessed as previously described (Weiss, R. H.; Nuccitelli, R. J. Biol. Chem. 267: 5608-5613; 1992).

Oligodeoxynucleotide transfections: Phosphorothioate antisense and random sequence control oligodeoxynucleotides were synthesized by Oligonucleotides Etc. (Wilsonville, Oreg.). The p21^{Waf1/Cip1} antisense vector was designed around the start codon of rat p21^{Waf1/Cip1}, with sequence 5′-GAC ATC ACC AGG ATC GGA CAT-3′ (SEQ. ID NO.:1). The scrambled random sequence control oligodeoxynucleotide was 5′-TGG ATC CGA CAT GTC AGA-3′(SEQ. ID NO.:3). For the lipofection procedure, cells were grown to 60% confluence, washed with sterile phosphate-buffered saline, and the appropriate concentration of oligodeoxynucleotide was mixed with 6.6 μL of Lipofectin® per ml of Opti-MEM medium and was added to the cells for 4 hours at 37° C. Serum-free medium (without oligodeoxynucleotide) was added overnight, the media was changed in the morning, and the cells were stimulated with TGF-β or serum as indicated.

Immunoprecipitations: VSM cells were grown to confluence. After incubation under appropriate conditions, the cells were washed with ice-cold phosphate-buffered saline and immediately lysed in lysis buffer (20 mM Tris [pH7.5], 150 mM NaCl, 1 mM EDTA, 1 mM EGTA, 1% Triton X-100, 2.5 mM sodium pyrophosphate, 1 mM β-glycerolphosphate, 1 mM Na₃VO₄, 1 μg/ml Leupeptin, 1 mM PMSF) at 4° C. The cells were scraped off with a rubber spatula and the insoluble material removed by centrifuging at 10,000× g for 10 min at 4° C. Protein concentration was determined by A₅₉₅, and lysates containing equal amounts of protein were incubated with 4 ml anti-fibronectin antibody at 4° C. overnight. Protein A-Sepharose beads were added and the resulting mixture was incubated for an additional 2 hours at 4° C. The beads were centrifuged in a microfuge for 20 sec, and the pellet was washed 3 times with cold lysis buffer. The supernatant was decanted, gel loading buffer was added to the precipitate, and the solution was boiled for 5 min, and centrifuged. The supernatant was electrophoresed on a 7.5% SDS-polyacrylamide gel with equal volumes of sample per lane. The proteins were electrophoretically transferred to nitrocellulose and probed with fibronectin antibody.

Western blots: Cells were grown to confluence in 6 cm culture dishes and serum deprived. After transfection and/or treatment with appropriate agonist, conditioned medium was removed and saved and the cells were washed with phosphate-buffered saline and lysed in lysis buffer. Both supernatant and cell lysate were normalized to the lysate protein concentrations and Western blotted as described (Weiss, R. H.; et al. Am. J. Physiol. 274: C1521-C1529; 1998).

### RESULTS

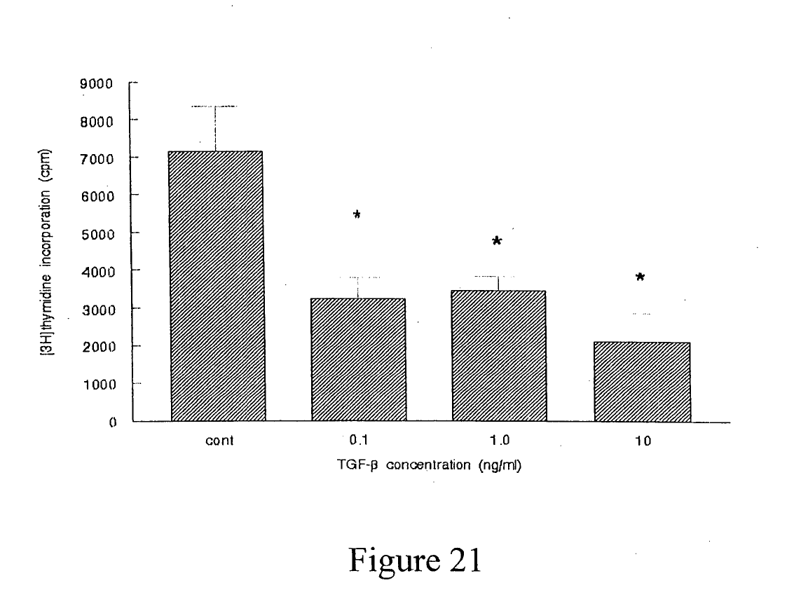

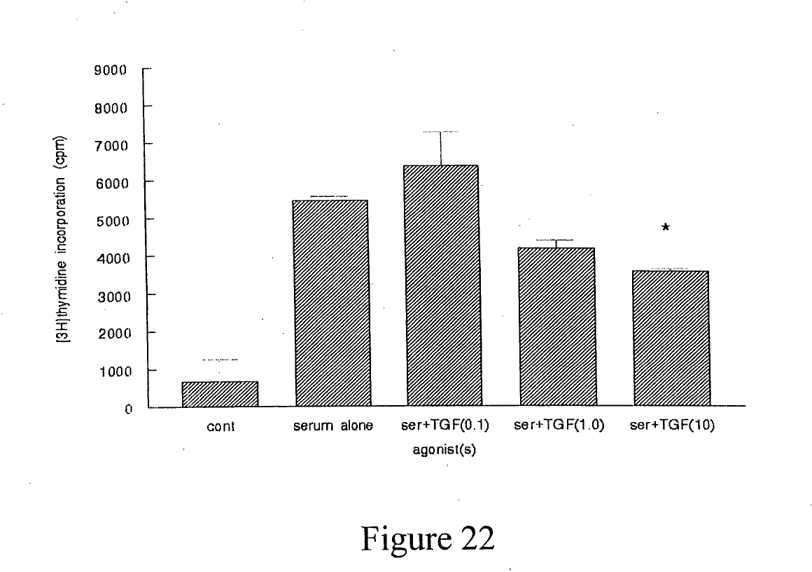

TGF-β has a bimodal effect on mitogenesis, being stimulatory or inhibitory depending on cell confluency and cell type (Moses, H. L.; et al. Cell 63: 245-247; 1990; and Centrella, M. et al. J. Biol. Chem. 262: 2869-2874; 1987). However, TGF-β is largely growth inhibitory in vivo (reviewed in Moses, H. L.; et al. Cell 63: 245-247; 1990). This property was confirmed in early passage rat VSM cells (Weiss, R. H.; et al. Kidney Int. 48: 738-744; 1995) and late passage rat mesangial cells (Weiss, R. H.; Ramirez, A. Nephrol. Dial. Transplant. 13: 2804-2813; 1998). To determine whether A10 VSM cells behave similarly, these cells were serum-starved for 24 hours prior to stimulation with from 0.1 to 10 ng/ml TGF-β, and their ability to incorporate [³H]thymidine into DNA was examined (expressed as mean ±s.e.m. of three wells per data point). At all concentrations tested, DNA synthesis was significantly reduced with the addition of TGF-β for 24 hours (FIG. 21). To determine whether this growth inhibitory effect persisted when the A10 VSM cells were stimulated to enter G₁ with the addition of serum, the effect of the addition of 10% serum-containing media on TGF-β-exposed cells at the same concentrations was examined and found to exhibit similar growth-inhibitory results (FIG. 22). DNA synthesis was assessed by [³H]-thymidine incorporation and is expressed as mean ±s.e.m. of three wells per data point; absolute counts differ slightly from other experiments due to differences in starting confluency of the cells.

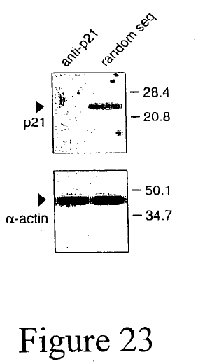

Using a random sequence phosphorothioate oligodeoxynucleotide (SEQ. ID NO.:3) as a control, the effect of the antisense oligodeoxynucleotide to p21 (400 nM) on p21 and α-actin protein expression in A10 VSM cells, was examined. The lysates, normalized for protein content, were Western blotted with either p21 or α-actin antibody. There was marked inhibition of p21 protein level, but no effect on the level of α-actin protein level, after transfection of 400 nM antisense p21 oligodeoxynucleotide (SEQ. ID NO.:1) (FIG. 23). Thus, this antisense oligodeoxynucleotide is specific in its inhibition of p21 protein expression and would not be expected to directly inhibit transcription or translation of matrix proteins which, of course, are unrelated in sequence to the CKIs.

1. Effect of Transfection of Antisense p21 (SEQ. ID NO.:1) or Random Sequence (SEQ. ID NO.:3) Oligodeoxynucleotide on TGF-β

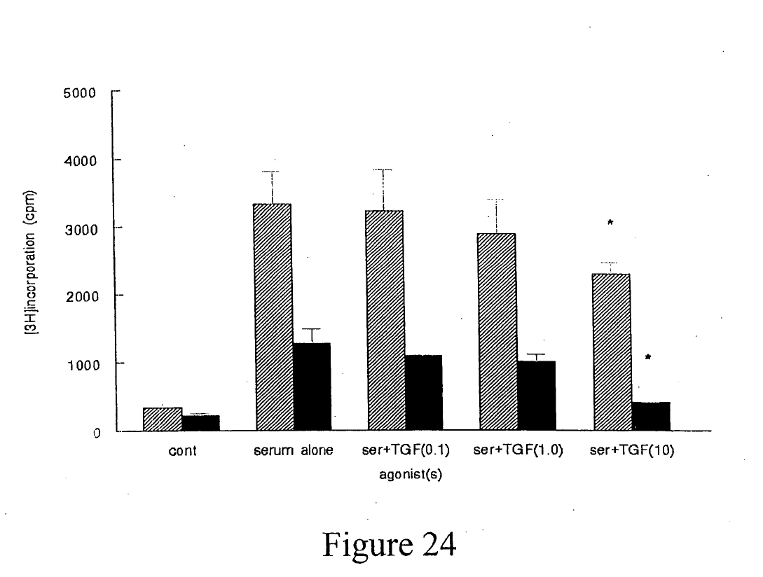

Cells were transfected with the appropriate oligodeoxynucleotide (antisense or random sequence) for 4 hours. The cells were then serum-starved overnight and subsequently stimulated with 10% serum-containing media. Two hours after stimulation with serum, TGF-β was added at concentrations from 0.1 to 10 ng/ml for 24 hours. While DNA synthesis of cells transfected with antisense p21 was markedly inhibited relative to cells transfected with control oligodeoxynucleotide (as it was previously shown when comparing antisense p21 with sense p21 oligodeoxynucleotide (Example 1, herein), the inhibitory effect of TGF-β at 10 ng/ml was still present under both conditions (FIG. 24).

2. Effect of p21 on Matrix Protein Secretion in VSM Cells

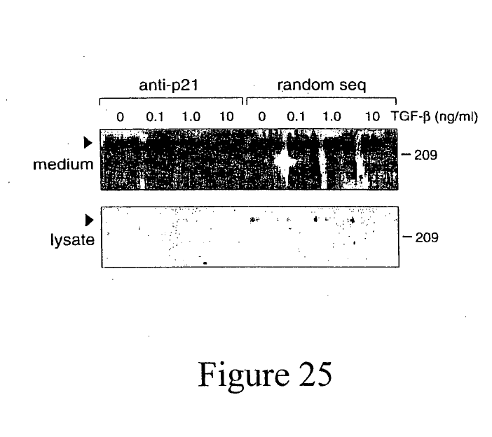

The p21 influences on matrix protein secretion in VSM cells were determined. Levels of the matrix proteins laminin and fibronectin were examined in both TGF-β stimulated lysate and conditioned media of cells in which p21 expression had been attenuated. The cells were transfected with either antisense p21 (SEQ. ID NO.:1) (400 nM), or random sequence control (SEQ. ID NO.:3) (400 nM) oligodeoxynucleotide, and then stimulated with TGF-β at for 0.1 to 10 ng/ml for 4 hours. Conditioned media and cell lysate were collected, and both medium and lysate volumes were normalized for the protein content in total cell lysate to exclude any skewing of the data due to cell proliferation. The proteins were electrophoresed and immunoblotted with either fibronectin (after immunoprecipitation to eliminate extraneous bands which appeared in the absence of this procedure) or laminin antibody. Production and secretion of laminin was markedly reduced after attenuation of p21, yet the effect of TGF-β was to decrease laminin secretion at higher doses in control oligodeoxynucleotide transfected cells (FIG. 25).

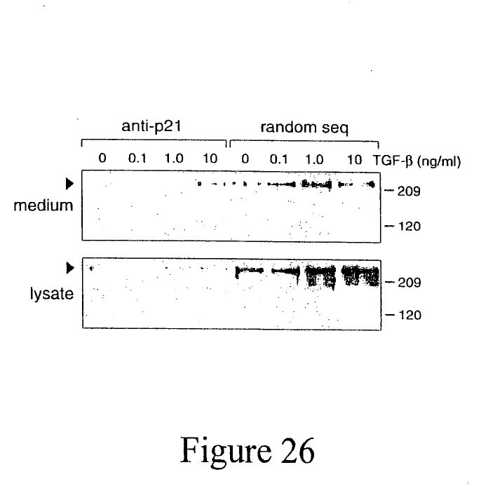

Fibronectin production and secretion into the medium was similarly decreased after p21 attenuation, yet, in this case, TGF-β induced fibronectin production with a maximal level in lysate when fibronectin was administered at higher doses (FIG. 26).

### DISCUSSION

The early lesions of atherosclerosis are associated with migration and proliferation of VSM cells. Once these cells enter the proliferative state, they attain a synthetic phenotype which causes them to secrete matrix proteins (Assoian, R. K.; Marcantonio, E. E. J. Clin. Invest 100: S15-S18; 1997; and Thyberg, J.; et al. Arteriosclerosis 10: 966-990; 1990). Exuberant secretion of these proteins may lead to fibrosis, but the same proteins may also regulate the cell phenotype and cause it to either remain secretory or become proliferative (Thyberg, J et al. J. Histochem. Cytochem. 45: 837-846; 1997).

Matrix proteins are secreted by a variety of cells and are important for structural integrity in the normal environment, yet these same proteins may be detrimental when they occur in abundance in the disease setting (reviewed in (Rizzino, A. Dev. Biol. 130: 411-422; 1988)). Furthermore, matrix proteins have been assigned the role of cell cycle control elements in atherosclerotic disease (Assoian, R. K.; Marcantonio, E. E. J. Clin. Invest 100: S15-S18; 1997). The specific matrix proteins laminin and fibronectin are important in modulating the switch from contractile to synthetic phenotype in VSM cells (reviewed in (Thyberg, J.; et al. Arteriosclerosis 10: 966-990; 1990)).

The extracellular matrix plays a key role in the progression of fibrosis in a variety of disparate diseases in multiple organ systems. In VSM and related glomerular mesangial cells, overexuberant secretion of matrix proteins is likely responsible for progression of atherosclerosis as well as of glomerular disease (reviewed in (Assoian, R. K.; Marcantonio, E. E. J. Clin. Invest 100: S15-S18; 1997; and Border, W. A.; Noble, N. A. N. Engl. J. Med. 331: 1286-1292; 1994)). The growth factor TGF-β, which in VSM cells is generally growth inhibitory (Weiss, R. H.; et al. Kidney Int. 48: 738-744; 1995; and Reddy, K. B.; Howe, P. H. J. Cell. Physiol. 156: 48-55; 1993), causes fibrosis in a variety of tissues and has been linked to an increase in matrix protein production as an etiology for this pathologic process (Border, W. A.; et al. Kidney Int. 37: 689-695; 1990; and Nakamura, T.; et al. Kidney Int. 41: 1213-1221; 1992), such that antibodies to TGF-β suppress arterial intimal hyperplasia and restenosis (Wolf, Y. G.; et al. J. Clin. Invest 93: 1172-1178; 1994) as well as experimental glomerulonephritis (Border, W. A.; et al. Nature 346: 371-374; 1990).

Studies have shown that the CKI p27 may mediate the switch from hyperplasia to the hypertrophic phenotype in VSM cells in response to TGF-β (Gibbons, G. H.; et al. J. Clin. Invest. 90: 456-461; 1992 Braun-Dullaeus, R. C.; et al. J. Clin. Invest. 104: 815-823; 1999)). Further, CKIs are important in regulating cell cycle transit (Sherr, C. J.; Roberts, J. M. Genes and Dev. 13: 1501-1512; 1999). Thus it was decided to examine whether the CKI p21 plays a role in the regulation of TGF-β-mediated matrix protein synthesis and secretion in VSM cells.

TGF-β is a growth factor that has variable influences on VSM and glomerular mesangial cells, the latter of which are modified smooth muscle cells. Depending on the cell type and culture conditions, TGF-β can be either stimulatory or inhibitory towards cell growth (Moses, H. L.; et al. Cell 63: 245-247; 1990). Despite its bimodal effect on cell proliferation, it is clear that TGF-β induces the pathologic appearance of matrix proteins, and thus this growth factor has been implicated as a causative agent in a variety of diseases which are characterized by fibrosis (Border, W. A.; Noble, N. A. N. Engl. J. Med. 331: 1286-1292; 1994; and Border, W. A.; et al. Kidney Int. Suppl 49: S59-S61; 1995). There is even evidence that fibronectin (Madri, J. A.; et al. J. Cell Biol. 106: 1375-1384; 1988) and laminin (Thyberg, J et al. J. Histochem. Cytochem. 45: 837-846; 1997) may actually be mediating the growth inhibitory effects of TGF-β. In any case, an understanding of the mechanism by which vascular cells attain the secretory phenotype, and can therefore be influenced by TGF-β to secrete matrix proteins, is of pivotal importance in the study of fibrotic diseases.

The results described herein show that p21 antisense oligodeoxynucleotides, which specifically inhibit p21 protein levels in these cells, markedly attenuate both synthesis and secretion of the matrix proteins fibronectin and laminin. These results in vascular cells have profound implications for treatment of fibrotic diseases, such as atherosclerosis and restenosis. Furthermore, since glomerular mesangial cells are modified VSM cells, the findings can be extended to treatment of glomerular diseases characterized by fibrotic changes mediated through matrix protein production.

The CKIs p21 and p27 have long been known to be induced by TGF-β (Polyak, K.; et al. Genes Dev. 8: 9-22; 1994; and Grau, A. M.; et al. Cancer Res. 57: 3929-3934; 1997), and it has been assumed that expression of those proteins links this growth factor to cell cycle arrest. It has been shown that in VSM, mesangial, and prostate carcinoma cells, p27 and p21 are induced by the growth inhibitory statins (Weiss, R. H.; Ramirez, A.; Joo, A. J. Am. Soc. Nephrol. 9: 1880-1890; 1999; Lee, S. J.; et al. J. Biol. Chem. 273: 10618-10623; 1998; and Terada, Y.; et al. J. Am. Soc. Nephrol. 9: 2235-2243; 1999). While these proteins likely in some manner cause attenuation of DNA synthesis in that setting, it has also been shown that p21 is required for the full mitogenic effect of PDGF and serum (Weiss, R. H.; et al. J. Biol. Chem. 275: 10285-10290; 2000; Weiss, R. H. and Randour, C. Cellular Signalling, 12:413-418, 2000), a correlation which has recently been confirmed by another group (Wakino, S, et al. J Biol Chem 275(9):22435-22441, 2000). Other investigators have shown the existence of a switch from a contractile to a synthetic phenotype in cells stimulated to proliferate (Thyberg, J.; et al. Arteriosclerosis 10: 966-990; 1990). The data presented in Example IV are consistent with this scenario, since decreased synthesis of matrix protein in cells transfected with antisense p21 was shown, concomitant with attenuation of proliferation in these cells.

Example IV demonstrates that the ability of TGF-β to synthesize and secrete the matrix proteins laminin and fibronectin, but not TGF-β's entire growth inhibitory effect, is mediated through p21. It was previously shown that, in mesangial cells, TGF-β at 10 ng/ml induced secretion of the matrix proteins fibronectin and laminin (Weiss, R. H.; Ramirez, A. Nephrol. Dial. Transplant. 13: 2804-2813; 1998). However, in the VSM cells used in the present experiments, higher concentrations of TGF-β were associated with decreased laminin synthesis and secretion into conditioned media. This may be due to the fact that continued stimulation of VSM cells by TGF-β causes them to remain in a synthetic phenotype associated with fibronectin secretion, whereas cells remain in the contractile phenotype when grown in the presence of the secreted laminin (Thyberg, J et al. J. Histochem. Cytochem. 45: 837-846; 1997).

It is noteworthy that transfection of antisense oligodeoxynucleotides has been shown to be effective in vivo both with and without lipofection reagents, and in some situations antisense oligodeoxynucleotides have even proved effective under conditions as simple as intravenous oligodeoxynucleotide infusions (Monia, B. P.; et al. Nat. Med. 2: 668-675; 1996). Therefore, these compounds may be useful clinically to target molecules important in fibrotic diseases such as, atherosclerosis, restenosis and glomerular disease.

It has been demonstrated that, in the case of mesangial cells, p21 is required for glomerular hypertrophy in experimental diabetic nephropathy (Al Douahji, M.; et al. Kidney Int. 56: 1691-1699; 1999). p21 maybe allowing secretion of matrix proteins in this scenario such that its inhibition would diminish this response. In VSM cells, p27 has been shown to have a similar role in the promotion of hypertrophy (Braun-Dullaeus, R. C.; et al. J. Clin. Invest. 104: 815-823; 1999), yet the role of p21 and matrix protein secretion in this phenomenon in VSM cells is not known. In the setting of angioplasty, VSM cells have been shown to modulate from a contractile to a synthetic phenotype after induction of intimal lesions by balloon catheterization (Grunwald, J.; et al. Exp. Mol. Pathol. 46: 78-88; 1987; and Manderson, J. A.; et al. Arteriosclerosis 9: 289-298; 1989). This may in turn result in excess production of matrix proteins, leading to ultimate restenosis of the vessel. Attenuation of matrix protein production and secretion with antisense p21 transfection into VSM cells may therefore prove to be useful, where such oligodeoxynucleotides could be lipofected into angioplastied blood vessels at the time of balloon catheterization.

A study was performed in cells from knockout mice, where it was shown that lack of a functional p21 gene ameliorated progression to chronic renal failure (Megyesi, J.; et al. Proc. Natl. Acad. Sci. U.S.A. 96: 10830-10835; 1999). In this study, none of the p21(−/−) mice developed glomerulosclerosis or interstitial fibrosis, as opposed to 70% of the glomeruli in p21 (+/+) animals, suggesting that p21 may be responsible for mediating a TGF-β effect on the cells leading to fibrosis by means of matrix protein secretion.

### EXAMPLE V

### In Vivo Studies of the Effect of Antisense Oligonucleotide of p21 on Met-1 Breast Cancer

Angiogenesis is the means by which growing tumors maintain oxygen necessary for their survival by means of their creation of auxiliary blood vessels. This is generally thought to result for the effect of various growth factors (such as VEGF) acting on endothelial cells. The possibility that angiogenesis may also be attenuated by inhibition of VSM cells is a possibility which has not been adequately investigated.

Materials and Methods: NZW mice were obtained and injected subcutaneously 2 days after their arrival with Met-1 breast cancer cells. Each mouse was injected on two sides of the breast area, such that two tumors arose in most animals. The following day, intraperitoneal injections at the indicated concentrations were made on a daily basis. When tumors appeared, they were measured in two dimensions using calipers and area of the tumors was used as rough measure of tumor mass. The tumor areas were averaged for each mouse and then these numbers were subsequently averaged. The general health of the mice was monitored on a daily bases as well.

Antisense Preparation: Phosphorothioate antisense oligodeoxynucleotides were synthesized by Oligonucleotides Etc. (Wilsonville, Oreg.). The p21^{Waf1/Cip1} antisense vector was designed around the start codon.of rat p21^{Waf1/Cip1}, with sequence 5′-GAC ATC ACC AGG ATC GGA CAT-3′ (SEQ. ID NO.:1). The sense p21^{Waf1/Cip1} sequence is 5′-ATG TCC GAT CCT GGT GAT GTC-3′ (SEQ. ID NO.:2). The scrambled random sequence control oligodeoxynucleotide was 5′-TGG ATC CGA CAT GTC AGA-3′ (SEQ. ID NO.:3).

Animal models of potential chemotherapeutics using the p21 antisense oligonucleotides of the invention were examined. Intraperitoneal injection of the oligonucleotides into mice previously injected with cells from a Met-i breast cancer line (Bourguignon, L. Y. et al, J. Cell Phys. 1998, 176, 206-215; Lau, D.H. et al., Cancer Biother. Radiopharm. 1999, 14, 31-6) were studied. The p21 antisense oligonucleotide or random sequence control oligonucleotide at a concentration of 0.6 mg oligonucleotide/kg mouse (Monia, B. P.; et al. , 1996, Nat. Med. 2: 668-675) were injected intraperitoneally daily when tumors first became palpable (day 1). There were 2 tumors per mouse (palpable in the breast area), and the tumors were measured in 2 dimensions by calipers at the times indicated. The areas of the 2 tumors per mouse were averaged and then these numbers were averaged over all 3 mice per data point and presented as the mean+/−s.e.m. Transfection of the antisense p21 oligonucleotides did not affect growth of Met-1 cells in culture, suggesting an anti-angiogenesis effect in vivo.

### EXAMPLE VI

### Use of Antisense Oligodeoxynucleotide to p21^{Waf1/Cip1} Causes Apoptosis in Human Breast Cancer Cells

Materials: Mouse monoclonal anti-recombinant full-length p21^{Waf1/Cip1} antibody and HeLa cell nuclear extract were obtained from Upstate Biotechnology Inc. (Lake Placid, N.Y.). Mouse monoclonal antihuman PTEN and antihuman P13K (P85) antibodies, rabbit polyclonal anti-MAPK (extracellular signal-regulated kinase 1) antibody, and A-431 WCL, Caki-1 WCL, and Jurkat WCL were obtained from Santa Cruz Biotechnology (Santa Cruz, Calif.). Mouse antihuman PARP purified antibody was obtained from BD Biosciences (San Diego, Calif.). For the immunohistochemistry, mouse monoclonal anti full-length p21 antibody was obtained from Calbiochem (San Diego, Calif.). Goat antimouse and goat antirabbit horseradish peroxidase-conjugated IgG were obtained from Bio-Rad (Richmond, Calif.). Lipofectin was obtained from Invitrogen Life Technologies, Inc. (Carlsbad, Calif.). ECL Western Blotting Detection Reagents were obtained from Amersham Biosciences (Buckinghamshire, United Kingdom). CaspACE Assay System was obtained from Promega (Madison, Wis.). The avidin-biotin complex kit and 3,3′-diaminobenzidine tetrahydrochloride were obtained from Vector Laboratories (Burlingame, Calif.). All other reagents, including mouse anti-α-actin monoclonal antibody, were from Sigma (St. Louis, Mo.).

Cell Culture and DNA Synthesis: T47D (ductal carcinoma) and MCF7 (adenocarcinoma) human breast cancer cell lines were obtained from the American Type Culture Collection. All cell lines were maintained according to vendors' recommendations. The cells were growth arrested by placing them in serum-free quiescence media and exposed to 10% serum-containing media as indicated. [³H]Thymidine incorporation was assessed as described previously (Weiss, R. H., and Nuccitelli, R., J. Biol. Chem., 267: 5608-5613, 1992).

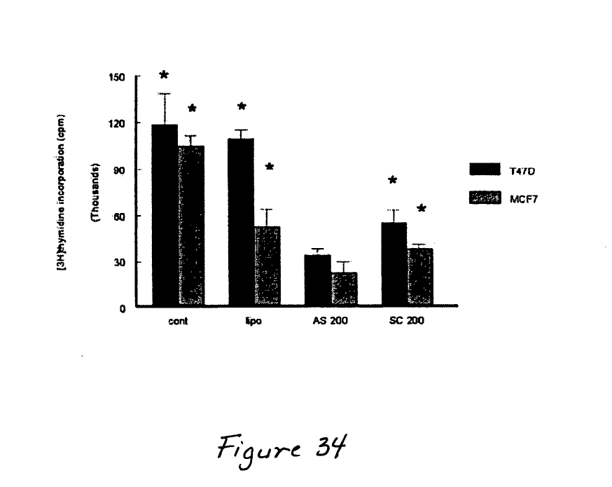

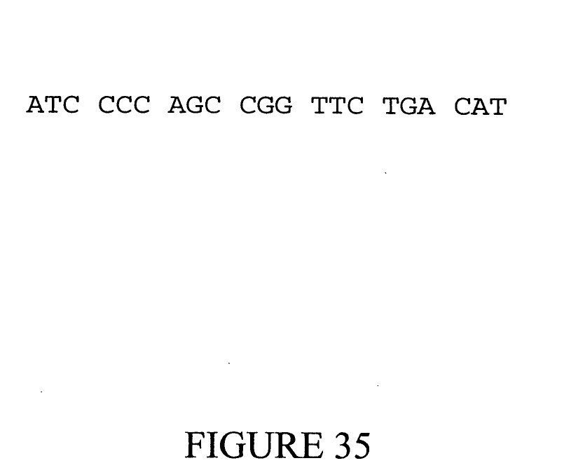

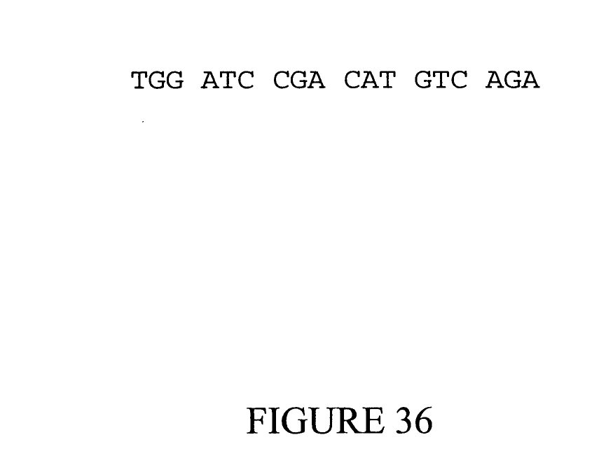

Antisense Transfections: Human p21^{Waf1/Cip1} antisense and control Oligodeoxynucleotides (ODNs) were synthesized by Oligos Etc. (Wilsonville, Oreg.). The human p21 antisense ODN sequence was 5′-ATC-CCC-AGC-CGG-TTC-TGA-CAT-3′ (SEQ ID NO.: 4). The randomly scrambled sequence of control ODN was 5′-TGG-ATC-CGA-CAT-GTC-AGA-3′ (SEQ ID NO.: 3), and the second control ODN (used for the experiment depicted in FIG. 34 was the sense human p21 sequence 5′-TAC-AGT-CTT-GGC-CGA-CCC-CTA-3′ (SEQ ID NO.: 5). For the transfection procedure, cells were grown to 70% confluence and washed with sterile PBS, and the ODNs (200 or 400 nM) were mixed with 6.6 μl Lipofectin/ml Opti-MEM media and added to the cells for 5 hours at 37° C. Serum-free media (without ODNs) were added overnight, the media were changed in the morning, and the cells were stimulated with serum or platelet-derived growth factor-BB as indicated.

RT-PCR: RT-PCR was performed as described previously (Weiss, R. H., and Howard, L. L., Cell Signalling, 13: 727-733, 2001), using primers of sequence 5′-ACC-TCA-CCT-GCT-CTG-CTG-C-3′ (sense) (SEQ ID NO.: 6) and 5′-GAC-TGC-AGG-CTT-CCT-GTG-G-3′ (antisense) (SEQ ID NO.: 7) corresponding to the NLS region of human p21. The antisense primer is the 3′-flanking region of the coding sequence, with a PCR product size of approximately 230 bp because that is a convenient size for separation by agarose gel electrophoresis. Because the NLS amplification product contains the border between the second and third exons, the cDNA was used as a template rather than genomic DNA, as is standard practice (van 't Veer, L. J., Dai, H., van de Vijver, M. J., He, Y. D., Hart, A. A., Mao, M., Peterse, H. L., van der Kooy, K., Marton, M. J., Witteveen, A. T., Schreiber, G. J., Kerkhoven, R. M., Roberts, C., Linsley, P. S., Bernards, R., and Friend, S. H. Nature (Lond.), 415: 530-536, 2002). The PCR products from all tumors and controls were sequenced at the University of California Davis Peptide Structure Laboratory.

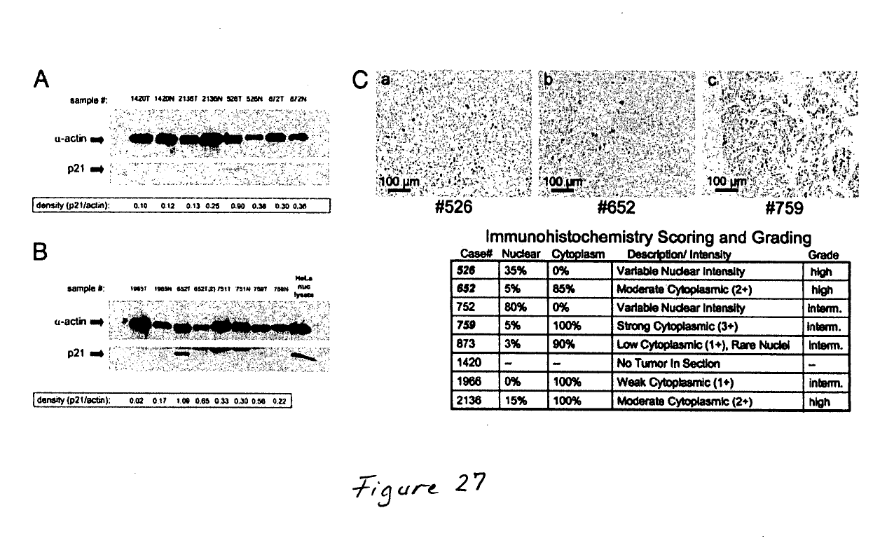

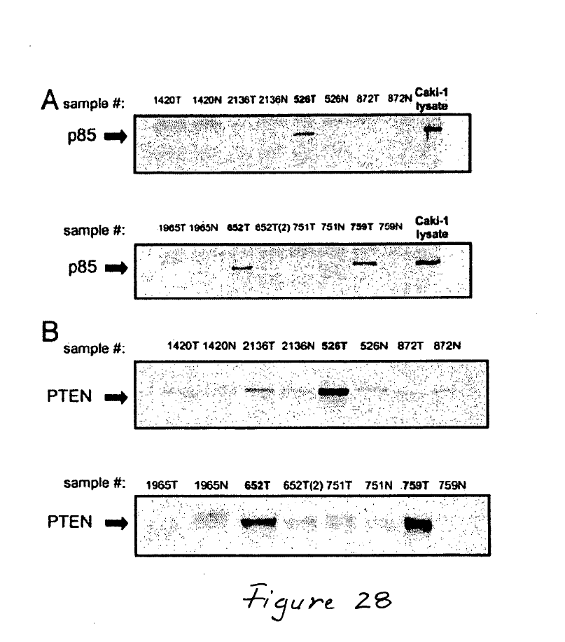

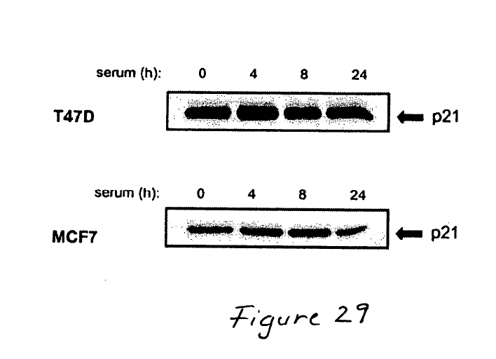

Immunoblotting: For FIGS. 27A and B, aliquots of each breast tumor (7) and corresponding normal (N) tissue were homogenized and subjected to immunoblotting with full-length p21 antibody and α-actin as a loading control. Density of p21 and actin bands was determined and reported as a ratio. HeLa cell nuclear extract was electrophoresed on the same gel as a positive control for p21 mobility. The samples of breast cancer tissues were cut into small pieces and rapidly homogenized in lysis buffer (1 ml/ mg) at 4° C. Cells from cell lines were grown to confluence in 60-mm culture dishes, and after incubation under appropriate conditions, the cells were washed with PBS and lysed in lysis buffer at 4° C. The tissue homogenates or cell lysates were centrifuged (13,000× g, 4° C., 10 min), and the supernatants were Western blotted as described previously (Weiss, R. H., Maga, E. A., and Ramirez, A., Am. J. Physiol., 274: C1521-C1529, 1998). Densitometry was analyzed using NIH Image.

Immunohistochemistry: For FIG. 27C, tissue blocks (where available) corresponding to all eight tumors were subjected to immunohistochemical analysis using full-length p21 antibody. Stained sections of the p21-positive tumors, as identified in FIG. 27A, are shown. Percentage of the tumor cells with cytoplasmic and nuclear p21 staining was estimated for all eight tumors. Percentage of cytoplasmic and nuclear staining, intensity of staining (using a scale of 0 to 3+), and tumor grading using a modified Bloom-Richardson combined histological grade were determined by a breast pathologist. The samples showing high levels of p21 by immunoblotting are pictured and indicated in bold (namely sample numbers: 526, 652 and 759). Formalin-fixed, paraffin-embedded tissue blocks of the human tumor samples were sectioned at 4-5-μm thickness, mounted on charged glass slides, baked for 1 hour at 60° C., deparaffinized, and rehydrated. Also for immunohistochemistry, cultured cells were grown and treated on chambered microscope slides (Bio-Tek Instruments, Winooski, Vt.). At the end point, cells were washed twice with ice-cold PBS and fixed with 4% neutral buffered formalin, dehydrated with 95% ethanol, and air dried at room temperature.

Slides were blocked and microwaved for antigen retrieval in 10 mM citrate (pH 6.0). Slides were incubated in full-length p21 (Calbiochem OP64) primary antibody solution in a humidified chamber overnight at room temperature, followed by incubation with secondary biotinylated antimouse antibody solution for 1 hour. The Vectastain ABC Elite Kit (Vector Laboratories) detection was performed according to the manufacturer's instructions. Slides were counterstained in Mayer's hematoxylin, dehydrated, cleared, and coverslipped. Slides were photographed with a Zeiss Axioskop light microscope and Axiocam digital camera.

Evaluation of Nuclear Morphology: Cells were seeded in 6-well culture dishes and treated with ODNs as described. The cells were then immersed in methanol for at least 10 min. Cells were stained in 1 μg/ml Hoechst 33258 in water with 1% nonfat dry milk. After staining for 8-10 min, the cells were rinsed in water and dried completely. Nuclear morphology was visually evaluated by fluorescence microscopy.

Caspase-3 Activity: The CaspACE Assay System was used to measure the activity of caspase-3. After incubation under appropriate conditions, the cells were lysed in CaspACE lysis buffer, and protein concentration was determined by dye reagent protein assay (Bio-Rad, Hercules, Calif.). Antisense ODN to p21 Causes Apoptosis in Breast Cancer The lysates with equal protein amounts were incubated with DEVD-p-nitroaniline substrate for 4 hours at 37° C. The reaction products were detected at 405 nm using a PowerWave X automated plate reader (Bio-Tek Instruments).

1. p21^{Waf1/Cip1} is Increased in Human Breast Cancers and is Associated with Aggressive Tumor Characteristics.

p21 has been demonstrated to be a negative regulator of p53-dependent and -independent apoptosis and to convey the survival signal of PI3K to downstream signaling pathways (Li, Y., Dowbenko, D., and Lasky, L. A., J. Biol. Chem., 277: 11352-11361, 2002; Zhou, B. P., Liao, Y., Xia, W., Spohn, B., Lee, M. H., and Hung, M. C., Nat. Cell Biol., 3: 245-252, 2001). However, studies on the prognostic role of p21 in a variety of cancers have not shown consistent results (O'Hanlon, D. M., Kiely, M., MacConmara, M., Al Azzawi, R., Connolly, Y., Jeffers, M., and Keane, F. B., Eur. J. Surg. Oncol., 28: 103-107, 2002; Winters, Z. E., Hunt, N. C., Bradbum, M. J., Royds, J. A., Turley, H., Harris, A. L., and Norbury, C. J., Eur. J. Cancer, 37: 2405-2412, 2001; Ceccarelli, C., Santini, D., Chieco, P., Lanciotti, C., Taffurelli, M., Paladini, G., and Marrano, D., Int. J. Cancer, 95: 128-134, 2001). Significantly, it has been shown that an antisense ODN to p21 attenuates growth of tumors in a mouse model of breast cancer (Weiss, R. H., Marshall, D., Howard, L., Corbacho, A. M., Cheung, A. T., and Sawai, E. T., Cancer Lett., 189: 39-48, 2003). The reports of additional investigators have shown involvement of p21 in cancer progression (Fan, S., Chang, J. K., Smith, M. L., Duba, D., Fomace, A. J., Jr., and O'Connor, P. M., Oncogene, 14: 2127-2136, 1997; Wouters, B. G., Giaccia, A. J., Denko, N. C., and Brown, J. M., Cancer Res., 57: 4703-4706, 1997; Barboule, N., Chadebech, P., Baldin, V., Vidal, S., and Valette, A., Oncogene, 15: 2867-2875, 1997) and cell survival (Li, Y., Dowbenko, D., and Lasky, L. A., J. Biol. Chem., 277: 11352-11361, 2002). As such, antisense techniques became a feasible means to study whether p21 is a feasible marker and target for future therapeutic intervention in human breast cancer.

Initial studies were directed at determining whether a subset of human breast cancers could be identified that were associated with activation of an antiapoptotic pathway. It was reasoned that, regardless of whether p21 levels directly correlate with patient outcome and in light of the antiapoptotic effect of intact p21, it is possible that (a) attenuation of p21 (as by antisense p21 ODN therapy) might result in a propensity of p21-attenuated cells to be more sensitive to the killing effects of DNA-damaging chemotherapy agents, and (b) identification of tumors expressing high levels of p21 might serve as a stratifier to identify patients who will respond to antisense p21 ODN treatment. In addition, it is possible that increased levels or cytosolic localization (vide infra) of p21 might be additionally useful in prognostication.

Breast cancers and corresponding control tissues from eight patients were obtained from the University of California Davis Human Biological Specimen Repository. Matched control tissues consisted of samples taken from each cancer biopsy that were chosen grossly and confirmed microscopically to be uninvolved by the tumor. Samples were numbered, and patient identifiers were stripped. Data collected for each sample were limited to the clinical pathological assessments of stage, grade, patient age, and routine marker studies. A portion of the cancer tissue from each biopsy sample as well as normal tissue from the same breast was homogenized, normalized for protein content, and subjected to immunoblotting for p21 using α-actin as a gel loading control. The immunoblot showed several nonspecific protein bands; p21 was identified using a positive control supplied by the antibody vendor (HeLa cell nuclear lysate), and levels of p21 normalized to α-actin were determined by densitometry. Increased p21 levels were seen in three of the tumor tissues (sample 526T, 652T, and 759T) and none of the normal tissues (labeled N, FIG. 27, A and B). Note that 652T did not have corresponding normal tissue; instead, two separate tumor samples were obtained. Pathology reports for tumors 526T and 652T gave a diagnosis of infiltrating ductal carcinoma, with a high combined histological grade of 3 (modified Bloom-Richardson, highest grade of 3). Both of these tumors had poor prognostic factors, including large tumor size, multiple lymph node involvement, and high grade. Tumor 759T, with the weakest p21 staining of the three positives by immunoblot, was also an infiltrating ductal carcinoma but had features of the mucinous carcinoma special type and had an intermediate combined histological grade of 2. No nodes were reported on sample 759. Grades of all of the tumor samples are shown in FIG. 27C.

It has been shown that intracellular localization of p21 plays a role in dictating its effect on cell cycle progression and apoptosis (Winters, Z. E., Hunt, N. C., Bradburn, M. J., Royds, J. A., Turley, H., Harris, A. L., and Norbury, C. J., Eur. J. Cancer, 37: 2405-2412, 2001; Li, Y., Dowbenko, D., and Lasky, L. A., J. Biol. Chem., 277: 11352-11361, 2002; Asada, M., Yamada, T., Ichijo, H., Delia, D., Miyazono, K., Fukumuro, K., and Mizutani, S., EMBO J., 18: 1223-1234, 1999; Zhou, B. P., Liao, Y., Xia, W., Spohn, B., Lee, M. H., and Hung, M. C., Nat. Cell Biol., 3: 245-252, 2001,) [As such it was next asked whether such localization correlated with the findings in the above studied tumors. p21 immunostaining confirmed high levels of p21 in the three p21-positive tumors; however, the localization of p21 in these samples was variably predominantly cytosolic or nuclear (FIG. 27C). Whereas tissue was available for Western blotting on tumor 1420, no tumor appeared on the section analyzed. These results indicate that the use of p21 as a prognostic marker may be feasible.

A possible explanation for the increased aggressiveness of the tumors showing high levels of p21 is mutation of the NLS region encompassing the AKT binding consensus sequence, such that AKT binding does not occur, AKT is constitutively active, or p21 fails to localize in the nucleus, any of which might provide a tumor survival advantage (Zhou, B. P., Liao, Y., Xia, W., Spohn, B., Lee, M. H., and Hung, M. C., Nat. Cell Biol., 3: 245-252, 2001; Rodriguez-Vilarrupla, A., Diaz, C., Canela, N., Rahn, H. P., Bachs, O., and Agell, N., FEBS Lett., 531: 319-323, 2002). There exist several reports of such mutations, one of which was reported in a single breast carcinoma (Balbin, M., Hannon, G. J., Pendas, A. M., Ferrando, A. A., Vizoso, F., Fueyo, A., and Lopez-Otin, C., J. Biol. Chem., 271: 15782-15786, 1996). To examine this possibility in the three tumors identified above as possessing high levels of p21, RT-PCR of the tumor and control tissue of these three samples using primers encompassing the NLS region was performed. In all of the three tumors expressing high levels of p21, there was a precise correlation with the GenBank human p21 sequence, eliminating the possibility that the mechanism of tumor aggressivity in these three patients was due to a mutation in the exon region corresponding to the NLS.

2. P13K-related Signaling Proteins are Increased in High p21^{Waf1/Cip1} Expressing Human Breast Cancers.

There exist several mitogenic signaling pathways whose activation is important to the process of oncogenic transformation. The PI3K pathway is replete with oncogenically important components (Testa, J. R., and Bellacosa, A., Proc. Natl. Acad. Sci. USA, 98: 10983-10985, 2001), being activated by the oncogenes HER2 and epidermal growth factor receptor. P13K thus functions as a “survival” protein, having antiapoptotic properties when activated. In addition, PTEN, which attenuates PI3K activity by dephosphorylation of the lipid second messenger phosphatidylinositol-3,4,5-P₃, a product of PI3K activity, finctions to attenuate the proproliferative effects of PI3K, such that PTEN-inactivating mutations are also oncogenic (Li, J., Yen, C., Liaw, D., Podsypanina, K., Bose, S., Wang, S. I., Puc, J., Miliaresis, C., Rodgers, L., McCombie, R., Bigner, S. H., Giovanella, B. C., Ittmann, M., Tycko, B., Hibshoosh, H., Wigler, M. H., and Parsons, R., Science (Wash. DC), 275: 1943-1947, 1997).

All eight of the breast tumors and corresponding control tissues described above were examined for levels of these signaling proteins by immunoblotting. The same quantity of lysate was loaded as described in FIG. 27A. The same protein quantity (20 μg) of lysates used in FIG. 27 was electrophoresed and immunoblotted with antibodies for (A) the p85 subunit of PI3K, using whole cell lysate of Caki-1 renal adenocarcinoma as positive control, and (B) PTEN. Tumors showing high p21 levels by immunoblotting are indicated in bold (namely tumor numbers: 526T, 652T and 759T). All three of the tumors that overexpressed p21 (but none of the control samples) overexpressed the p85 catalytic subunit of P13K (FIG. 28A). Surprisingly, all three tumors with high p21 expression (but none of the other tumor or control samples) also expressed high levels of PTEN (FIG. 28B); however, the magnitude of the increases in p85 and PTEN did not correlate in all cases with the levels of p21. PTEN induction may be occurring in a counter-regulatory or homeostatic fashion, as an attempt by the cell to subvert the antiapoptotic effect of p21 through PI3K.

3. Antisense Oligonucleotide to p21^{Waf1/Cip1} Attenuates p21^{Waf1/Cip1} Levels and Causes Apoptosis in Two Human Breast Cancer Cell Lines.

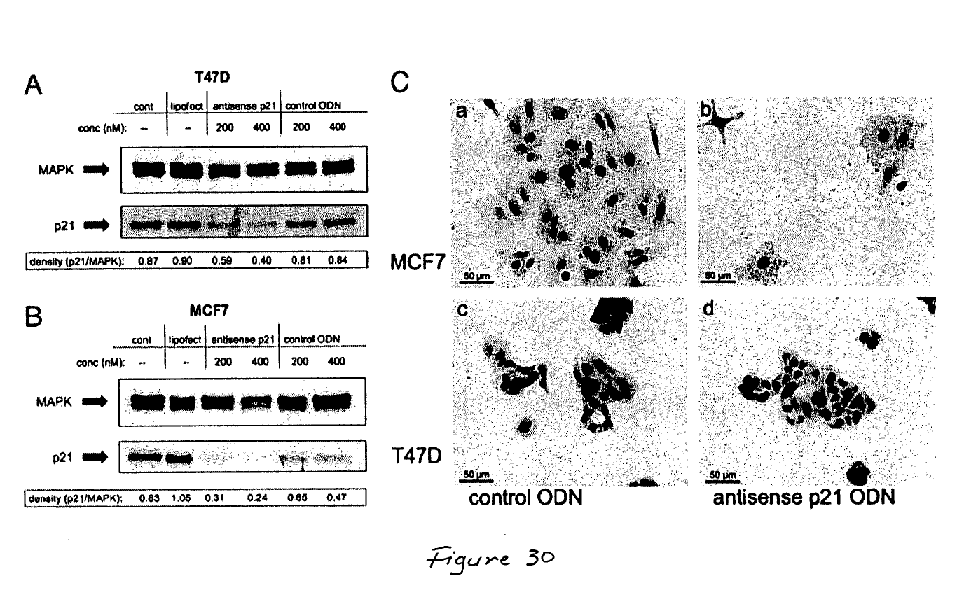

It has been shown previously that attenuation of p21 in a mouse model of breast cancer results in attenuation of growth of implanted Met-1 cells (which are derived from a high metastatic potential tumor in transgenic mice expressing polyomavirus middle T oncogene; (Weiss, R. H., Marshall, D., Howard, L., Corbacho, A. M., Cheung, A. T., and Sawai, E. T., Cancer Lett., 189: 39-48, 2003; Cheung, A. T. W., Young, L. J. T., Chen, P. C. Y., Chao, C. Y., Ndoye, A., and Barry, P. A. M., Int. J. Oncol., 11: 69-77, 1997)), likely due to induction of apoptosis in the tumor or blood vessels supplying it by this ODN. To begin to translate this work to human breast cancer, two human breast cancer cell lines, T47D (ductal carcinoma) and MCF-7 (adenocarcinoma), were studied for basal levels of p21. Surprisingly, both of these cell lines, whether serum starved or grown in serum-containing medium, displayed constitutively elevated levels of p21 (FIG. 29); compare with MAPK levels in control lanes of FIG. 30, in which the same quantity of protein was loaded), a property that may explain their transformed phenotype. For FIG. 29, T47D and MCF7 cells were grown to confluence in serum-containing media, serum starved for 24 hours, and then serum stimulated for the times indicated. The cells were lysed, and equal protein quantities (20 μg) were subjected to immunoblotting with full-length p21 antibody.

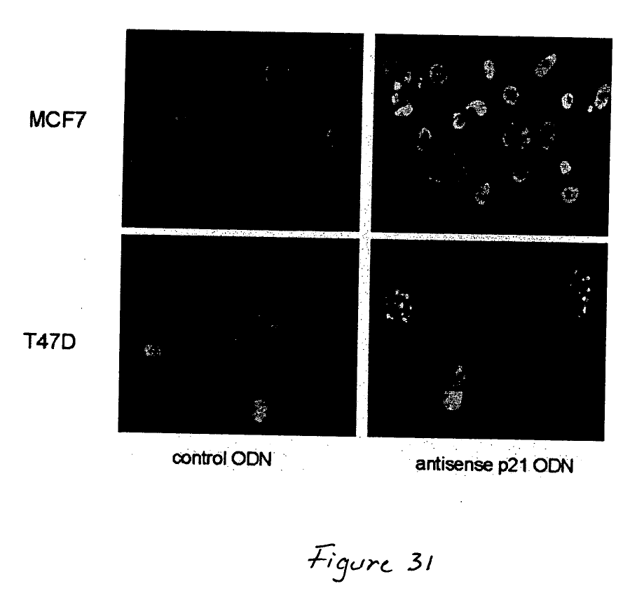

Due to the high levels of p21 in these two human breast cancer cell lines, it was reasoned that, in a manner similar to their effect in VSM cells (Weiss, R. H., Joo, A., and Randour, C., J. Biol. Chem., 275: 10285-10290, 2000), antisense ODNs to p21 may result in increased apoptosis and/or cell cycle arrest. This phenomenon might explain the effects of the antisense p21 ODN that were observed in nude mice and suggest future therapeutic possibilities for the use of this ODN in human disease. T47D and MCF7 cells were lipofected with either antisense p21 ODN (200 or 400 nM), the control ODN at the same concentrations, or lipofectin alone (no DNA). MCF7 and T47D cells were grown to confluence, serum starved for 24 hours, and lipofected with antisense p21 or control ODN or lipofectin only. cont refers to nontransfected cells. The cells were subsequently serum stimulated for another 24 hours. A and B, the cells were lysed, and equal protein quantities (20 μg) were electrophoresed and immunoblotted with p21 antibody. The same lysate was immunoblotted with MAPK antibody as a loading control. Density of p21 and MAPK bands was determined and reported as a ratio. C, the cells were transfected as described in A, fixed, subjected to immunohistochemistry with p21 antibody as described in “Immunohistochemistry” in Example VI, and examined under visible light at X400. Control cells (FIG. 30, cont) were not transfected. When examined 24 hours after transfection, both cell lines showed marked, dose-dependent attenuation of p21 (FIG. 30, A and B). Whereas there was a moderate decrease in p21 in the MCF7 cells after transfection with the control ODN, dose-dependent attenuation of p21 in response to antisense p21 ODN was substantially greater; this effect parallels the apoptotic effect of two control ODNs (see FIGS. 32 and 34) and is likely due to exquisite sensitivity of the MCF7 cells to ODN transfection. There was no effect of lipofectin alone when compared with no transfection (control). Specificity of antisense p21 ODN to p21 has been shown in prior studies (Weiss, R. H., Joo, A., and Randour, C., J. Biol. Chem., 275: 10285-10290, 2000; Hupfeld, C. J., and Weiss, R. H., Am. J. Physiol. Endocrinol. Metab., 281: E207-E216, 2001; Wong, G. A., Tang, V., El Sabeawy, F., and Weiss, R. H., Am. J. Physiol. Endocrinol. Metab., 284: E972-E979, 2003) and is further supported by the lack of modulation of MAPK (or PARP; see FIG. 32) levels in the ODN-transfected cells as compared with controls. However, an inhibitory effect of the control ODN on p21 levels occurred in both cell lines (although it was more pronounced in the MCF-7 cells), likely due to a nonspecific toxic effect of ODN transfections in general in these cells.

To determine whether a decrease in total p21 levels by immunoblotting parallels changes in cellular levels of p21 in breast cancer cells, immunohistochemistry of both T47D and MCF7 cells after transfection with the antisense p21 ODN was performed. The MCF7 cells become sparse after transfection with the antisense p21 ODN (FIGS. 30C, a and b), as expected given their increased propensity to apoptosis. Both the MCF7 and the T47D cells showed decreased p21 staining in the antisense p21 transfected cells as compared with the random sequence ODN-transfected cells (FIG. 30C), consistent with the immunoblots of these cells (FIG. 30, A and B).

Because cancer is characterized by disordered cell cycling, it is reasonable to assume that p21, which carries out the growth-arresting orders of p53 as well as the proproliferative effects of some mitogens, may play a role in the origin or progression of this disease and its response to conventional treatment. Furthermore, if this assumption is correct, it follows logically that manipulation of this protein, as with antisense ODNs, may be of use in cancer therapy. In fact, decreasing p21 has been shown to trigger apoptosis in human cancer cells likely by disallowing faithful repair of damaged DNA (Polyak, K., Waldman, T., He, T. C., Kinzler, K. W., and Vogelstein, B., Genes Dev., 10: 1945-1952, 1996; Gorospe, M., Cirielli, C., Wang, X., Seth, P., Capogrossi, M. C., and Holbrook, N. J., Oncogene, 14: 929-935, 1997), and the use of antisense ODNs against other targets has already shown promise in treatment of breast cancer (Head, J. F., Elliott, R. L., and Yang, D. C., Expert Opin. Ther. Targets, 6: 375-385, 2002). The current dogma of cancer chemotherapy is that these drugs ultimately generate signals that activate or open apoptotic metabolic pathways (Elledge, R. M., and Allred, D. C., Breast Cancer Res. Treat., 52: 79-98, 1998; Lowe, S. W., Bodis, S., McClatchey, A., Remington, L., Ruley, H. E., Fisher, D. E., Housman, D. E., and Jacks, T., Science (Wash. DC), 266: 807-810, 1994); thus, because p21 influences the outcome of the p53 response to cell damage from these proapoptotic agents (cell cycle arrest versus apoptosis; (Seoane, J., Le, H. V., and Massague, J., Nature (Lond.), 419: 729-734, 2002), it was next assessed whether attenuation of p21 results in apoptosis in the two breast cancer cell lines.

Apoptosis is the end result of an elaborate cascade of molecular events that ultimately result in programmed cell death; thus, a variety of tests are generally used to determine whether apoptosis is occurring. The methods to study apoptosis were employed: (a) assessment of nuclear morphology; (b) PARP cleavage; and (c) caspase-3 cleavage. Serum-starved cells were transfected with antisense p21 ODN or the control ODN, fixed, stained with Hoechst 33258, and examined under UV light. MCF7 and T47D cells were grown to confluence, serum starved for 24 hours, and lipofected with antisense p21 or control ODN. The cells were subsequently serum stimulated for another 24 hours, fixed, stained with Hoechst 33258, and examined under UV light at X400. Both MCF7 and T47D cells showed nuclear morphological changes consistent with apoptosis (FIG. 31) in response to antisense p21 ODN.

As a further measure of apoptosis, cleavage of PARP was next examined. This protein is cleaved by caspases and results in appearance of Mᵣ 85,000 and Mᵣ 24,000 fragments, the latter of which binds irreversibly to broken ends of DNA, which assures irreversibility of apoptosis. Both breast cancer cell lines were transfected with antisense p21 and random sequence ODNs and immunoblotted with PARP antibody. There was marked PARP cleavage, as evidenced by appearance of the Mᵣ 85,000 cleavage product, in the cells transfected with antisense p21 ODN (FIG. 32). T47D (A) and MCF7 (B) cells were grown to confluence, serum starved for 24 hours, and lipofected with antisense p21 or control ODN at the concentrations indicated. cont refers to nontransfected cells, and lipofect refers to cells treated with lipofectin but no DNA. The cells were lysed, and equal protein quantities were electrophoresed and immunoblotted with PARP antibody. The degradation product of PARP (Mᵣ 85,000) is indicated by an arrow. MAPK is a loading control; a positive PARP cleavage control is Jurkat whole cell lysate.

Similar to what was seen in FIG. 30, a proapoptotic effect of the control ODN on p21 levels was seen in both cell lines, likely due to a nonspecific toxic effect of ODN transfections in these cells. Indeed, it is possible that the proapoptotic effect of the control ODNs is a consequence of their inhibitory effect on p21 levels (FIG. 30), given what is known about the finction of p21 as a survival protein.

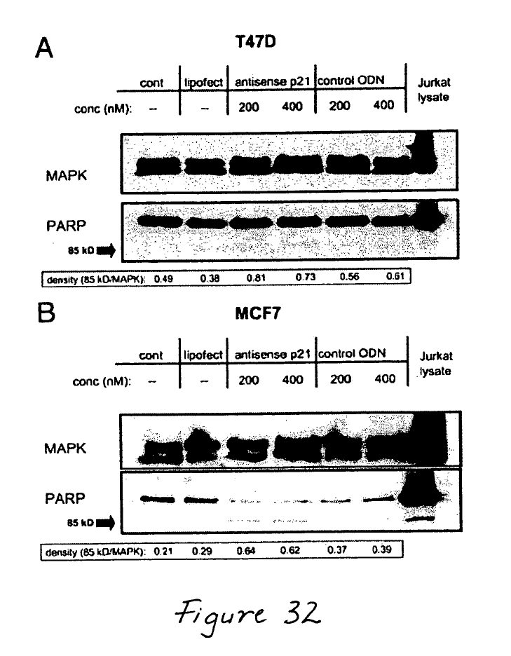

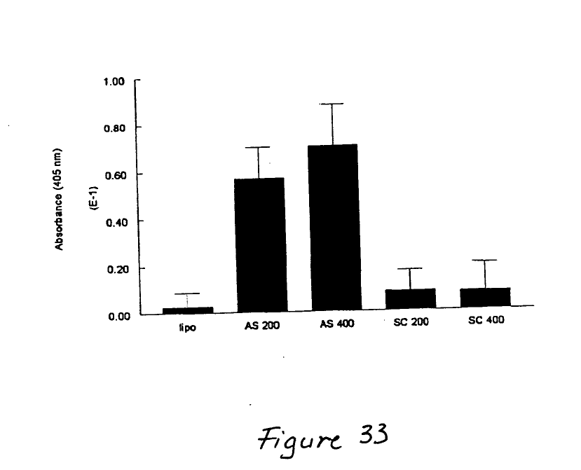

As another confirmatory test for antisense p21 ODN-mediated apoptosis, caspase-3 activation was assessed. Members of the caspase family of proteases are essential components of an evolutionarily conserved cell death pathway in multicellular eukaryotes and play key roles in inflammation and apoptosis in mammalian cells. Caspase-3 has substrate specificity for the amino acid sequence DEVD (Asp-Glu-Val-Asp) and is inhibited by the tetrapeptide inhibitor Ac-DEVD-CHO; its catalytic activity was assessed colorimetrically using the labeled Ac-DEVD-pNA substrate. T47D cells, after transfection with the antisense p-nitroaniline p21 ODN, showed marked dose-dependent caspase-3 cleavage, with no change after control ODN transfection (FIG. 33). The MCF-7 cells lack caspase-3 and thus did not show significant caspase-3 cleavage products. T47D cells were grown to confluence, serum starved for 24 hours, and lipofected with antisense p21 or control ODN at the concentrations indicated. Caspase-3 activity was assessed as described in “Capase-3 Activity” using a colorimetric method. AS 200 represents antisense p21 ODN transfected at 200 nM; SC 400 represents control ODN at 400 nM.

To further demonstrate apoptosis in the breast cancer cells in response to antisense p21 ODN, [³H]thymidine incorporation was next assessed in cells transfected with the ODNs (FIG. 34). Apoptotic cells will not incorporate thymidine into DNA during S phase, but [³H]thymidine incorporation experiments will not distinguish between G₁→S arrest and apoptosis. Serum-starved cells were transfected with 200 nM antisense p21 and control ODN, stimulated with serum-containing media, and examined for their ability to incorporate [³H]thymidine into DNA. Due to the significant attenuation of [³H]thymidine incorporation in control ODN-transfected cells, two different control ODNs were used: (a) the scrambled sequence ODN used in the preceding experiment; and (b) another control ODN that had the sense p21 sequence. Antisense p21 attenuates [³H]thymidine incorporation. MCF7 and T47D cells were grown to confluence, serum starved for 24 hours, and lipofected with antisense p21 (AS) or control (SC) ODN at the concentrations indicated (in nM). Control cells (cont) were not lipofected, and some cells (lipo) did not receive ODN. Serum was added for 24 hours, and the cells were incubated with [³H]thymidine for the last 6 hours. DNA synthesis was assessed by [³H]thymidine incorporation and is expressed as mean +SD of 3 wells/data point. *, P <0.05 compared with antisense p21 ODN transfection. This experiment was repeated with two separate control ODNs; a representative experiment using the sense control is shown.

In both cell lines, antisense p21 ODN transfection resulted in significant attenuation of DNA incorporation as compared with both control ODNs at 200 nM (FIG. 34). Both control ODNs (only one is shown in FIG. 34) showed similar attenuation, suggesting that these cancer cells are extremely sensitive to small ODN transfections or to p21 attenuation (compare with FIGS. 30 and 32). This thymidine data show a marked resemblance to the PARP cleavage data (FIG. 32); whereas a decrease in DNA synthesis can be the result of growth arrest, it is more likely, based on the apoptotic data shown above, that it is due in this case to promotion of apoptosis. Thus, antisense p21 ODN transfection results in apoptotic changes in two human breast cancer cell lines, in the absence of a chemotherapeutic stimulus. These results suggest that this ODN may be of therapeutic benefit in human disease.

### EXAMPLE VII

### Attenuation of p21^{Waf1/Cip1} Causes Apoptosis in Rat Aortic Vascular Cells Materials and Methods

Materials: Human recombinant PDGF-BB, mouse monoclonal full-length p21^{Waf1/Cip1}, and HeLa cell nuclear extract were obtained from Upstate Biotechnology (Lake Placid, N.Y.). Rabbit polyclonal human C-19 p21 antibody was obtained from Santa Cruz Biotechnology (Santa Cruz, Calif.). For the immunohistochemistry, mouse monoclonal full-length p21 was obtained from Calbiochem (San Diego, Calif.). Anti-goat horseradish peroxidase-conjugated IgG was obtained from BioRad (Richmond, Calif.). LipofectinR was obtained from Life Technologies (Rockville, Md.) and Fugene 6 from Roche (Indianapolis, Ind.). Reagents for the enhanced chemiluminescence system were obtained from Amersham (Arlington Heights, Ill.). The avidin-biotin complex (ABC) kit and 3,3-diaminobenzidine (DAB) tetrahydrochloride were obtained from Vector Laboratories (Burlingame, Calif.). All other reagents, including mouse monoclonal α-actin antibody, were from Sigma (St. Louis, Mo.).

Cell Culture, DNA Synthesis and Proliferation Assays: Cultures of A10 rat aortic vascular smooth muscle (VSM) cells were obtained from American Type Culture Collection (Rockville Md.), were maintained as described (Weiss R H, Maga E A, Ramirez A. Am J Physiol 1998;274:C1521- 9), and were used between passages 15 and 25. The cells were growth-arrested by placing them in serum-free quiescence medium and exposed to growth factors as indicated.

Immunoblotting: Cells from cell lines were grown to confluence in 60-mm culture dishes and after incubation under appropriate conditions, the cells were washed with phosphate-buffered saline and lysed in lysis buffer at 4° C. The tissue homogenates or cell lysates were centrifuged (13,000× g, 4° C., 10 min) and the supernatants were Western-blotted as previously described (Weiss R H, Maga E A, Ramirez A. Am J Physiol 1998;274:C1521-9). Densitometry was analyzed using NIH Image.

Plasmid Construction and Transfection: Construction of all plasmids, gifts from Dr. M. Asada, has been described (Asada M, Yamada T, Ichijo H, Delia D, Miyazono K, Fukumuro K, et al. EMBO J 1999;18:1223-34). A10 smooth muscle cells were transfected with pMTCB6+ (control vector), pMTCB6-ΔNLSp21 or pMTCB6-p21-full by Fugene 6. The transfected stable clones were selected with G418 at a concentration of 600 μg/ml. The expression of p21 protein in the transfected clones was confirmed by immunoblot analysis, in the presence of ZnSO₄ for 24 hours at a concentration of 30 to 120 μM.

Immunohistochemistry: For immunohistochemistry, VSM cells were grown and treated on chambered microscope slides (BIO-TEK Instruments, Winooski, Vt.). Stable VSM clones overexpressing pMTCB6+ (empty vector), pMTCB6-ΔNLS-p21 clones (NLS-deficient p21 clones 1, 7 and 9) or pMTCB6-p21-full (full-length p21) were incubated with ZnSO₄. for 24 hours at 0, 60 or 120 μM concentrations. The cells were fixed and stained with full-length p21 antibody (Calbiochem #OP64) and viewed at 400×. p21 is represented by the brown color and the nuclear counterstain is blue. At endpoint, cells were washed twice with ice cold PBS and fixed with 4% neutral buffered formalin, dehydrated with 95% ethanol and air-dried at room temperature. Slides were blocked and microwave treated for antigen retrieval in 10 mM citrate (pH 6.0). Slides were incubated in full-length p21 (Calbiochem #OP64) primary antibody solution in a humidified chamber overnight at room temperature, followed by secondary biotinylated anti-mouse antibody solution for 1 hour. The Vectastain ABC Elite Kit (Vector Laboratories) detection was performed according to manufacturer's instructions. Slides were counterstained in Mayer's hematoxylin, dehydrated, cleared and coverslipped. Slides were photographed with a Zeiss Axioskop light microscope and Axiocam digital camera.

1. Transfection of a Nuclear-Localization Signal Deficient (ANLS) p21 Construct Into Vascular Smooth Muscle Cells Results in Increased Cvtosolic Localization of p21.

Since p21 talks to various cytosolic (e.g. AKT) as well as nuclear (e.g. cdks and cyclins) proteins and, because this protein finctions in a pleiotropic manner depending on cell type and growth conditions, it is likely that the intracellular location of p21 plays a role in the regulation of its activity. p21 is directed to the nucleus by virtue of its possessing a nuclear localization sequence (NLS) at its C-terminus (Rodriguez-Vilarrupla A, Diaz C, Canela N, Rahn H P, Bachs O, Agell N. FEBS Lett 2002;531:319- 23). It has been previously shown that monocytes transfected with a NLS-deleted (ΔNLS-p21) construct resulted in cytosolic localization of this protein, protection from apoptosis and resistance to cell cycle arrest (Asada M, Yamada T, Ichijo H, Delia D, Miyazono K, Fukumuro K, et al. EMBO J 1999; 18:1223-34). The following experiments show that the same can be said where the transfected cell type is rat aortic VSM cells.

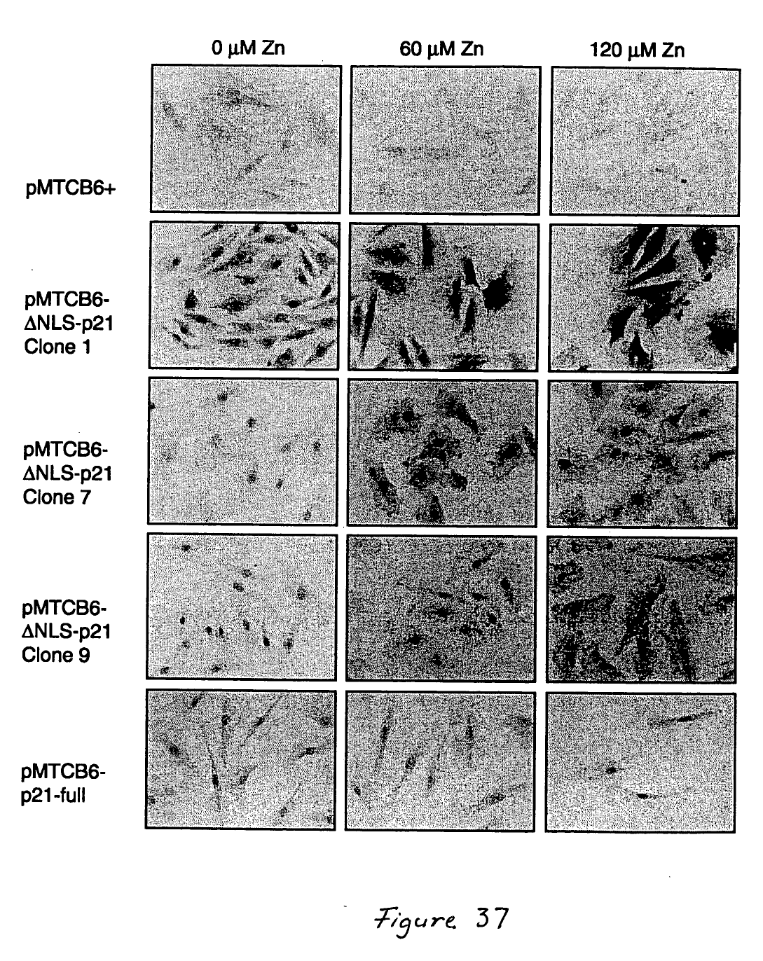

Rat aortic VSM cells were stably transfected with the pMTCB6− ΔNLS-p21 (NLS-deficient p21 with only amino acids 1- 140 under control of a Zn-responsive promoter), pMTCB6- p21-full (p21 with amino acids 1-164), or pMTCB6+ (empty vector) and selected using standard techniques. Cells randomly growing in serum-containing medium were exposed to ZnSO₄ at 60 to 120 μM for 24 hours, and fixed and stained for p21 as described in Materials and Methods above. Nine clones were selected and screened by immunohistochemistry for cytosolic localization of p21. Three of the nine clones transfected with pMTCB6-ΔNLS-p21 (clones 1, 7 and 9) showed markedly increased cytosolic localization of p21, which was increased with Zn addition (FIG. 37). In clone 1, while the addition of Zn increased cytosolic levels of p21, there was an increase in cytosolic staining in these cells compared to the pMTCB6+ (empty vector) transfected cells even in the absence of Zn, suggesting some leakiness of the MT promoter in this clone.

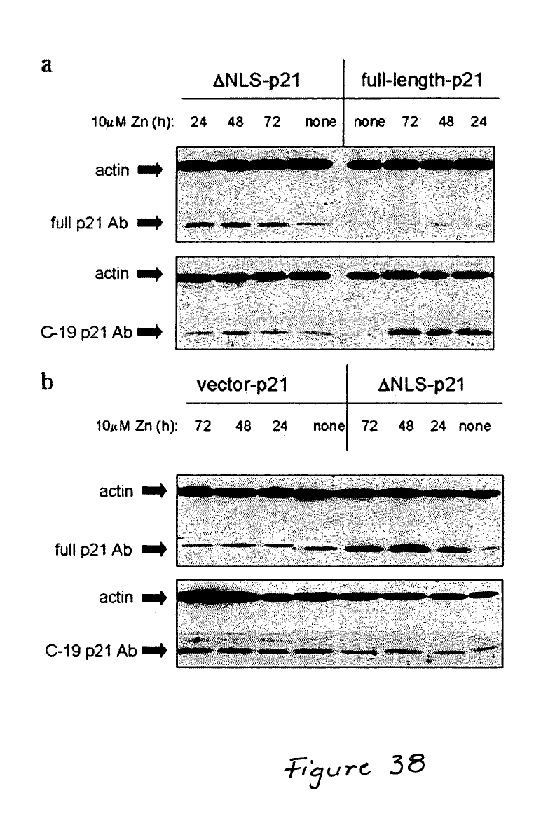

To confirm the presence and Zn responsiveness of the ΔNLS-p21 construct in transfected cells, immunoblotting was performed using both C-terminal (Santa Cruz, C-19) and full-length (Upstate Biotech, #05-345) human p21 antibodies in clone 9 of the pMTCB6-ΔNLS-p21 transfectants as well as pMTCB6+ (empty vector) and pMTCB6-p21-full (full-length p21). The full-length p21 antibody recognized transfected p21 in all of the clones, in addition to endogenous p21, and levels of this protein were induced in the presence of Zn in all except the empty vector-transfected cells, where a stable level of endogenous p21 was seen with both antibodies (FIG. 38). Overexpression of ΔNLS is confirmed using differential p21 antibodies. (a) NLS-deficient p21 transfected cells (pMTCB6-ΔNLSp21; clone 9) and full-length p21 transfected cells (pMTCB6-p21-full) were incubated with 10 μM ZnSO₄ for the times indicated. Cell lysate was normalized for protein and immunoblotted with full length (full p21 Ab) or C-terminal (C-19 p21 Ab) p21 antibodies. α-actin was immunoblotted as a loading control. (b) Empty-vector transfected cells (pMTCB6+) and NLS-deficient p21 transfected cells (pMTCB6-ΔNLSp21; clone 9) were incubated with ZnSO₄ and immunoblotted as described above. These experiments are representative of at least three separate experiments.

The maximal induction time was 48 hours of Zn, although there was little change from 24 to 72 hours of Zn incubation (FIG. 38b). When immunoblotted with the C-terminal p21 antibody, the ΔNLS-p21 transfected cells show staining similar to the vector (i.e. endogenous p21) with no induction by Zn, as expected since the C-terminal amino acids are missing in the ΔNLS-p21 construct (FIG. 38). It was further observed that there was a slightly increased level of p21 as measured by the full-length antibody in the ΔNLS cells as compared to the full-length p21 cells (FIG. 38a), again suggesting a slightly leaky MT promoter and consistent with the findings in the immunohistochemical analysis of all three pMTCB6-ΔNLSp21 clones (FIG. 37). Thus, cells transfected with these different vectors behave as expected in terms of cytosolic localization as well as selective epitope expression.

2. Transfection of a Nuclear-Localization Signal Deficient (ANLS) p21 Construct into Vascular Smooth Muscle Cells Causes Increased Cell Cycle Transit as Measured by [³H]thymidine Incorporation.

It has previously been shown that nuclear localization of p21 is required for growth suppression in certain breast cancer (Zhou BP, Liao Y, Xia W, Spohn B, Lee M H, Hung M C. Nat Cell Biol 2001;3:245 -52) and NIH3T3 (Rodriguez-Vilarrupla A, Diaz C, Canela N, Rahn H P, Bachs O, Agell N. FEBS Lett 2002;531:319- 23) cells, and others have shown that forced cytosolic localization of p21 results in apoptotic resistance and the absence of cell cycle arrest in monocytes (Asada M, Yamada T, Ichijo H, Delia D, Miyazono K, Fukumuro K, et al. EMBO .J 1999;18:1223-34). Since it had also been shown that attenuation of p21 leads to inhibition of cell cycle transit (Weiss R H, Joo A, Randour C. J Biol Chem 2000;275:10285- 90) in VSM cells and apoptosis in human breast cancer cells (Fan Y, Borowsky A D, Weiss R H. Mol Cancer Ther 2003;2:773- 82), experiments were next designed to explore whether a complementary positive effect of cytosolically localized p21 on cell cycle transit could be demonstrated.

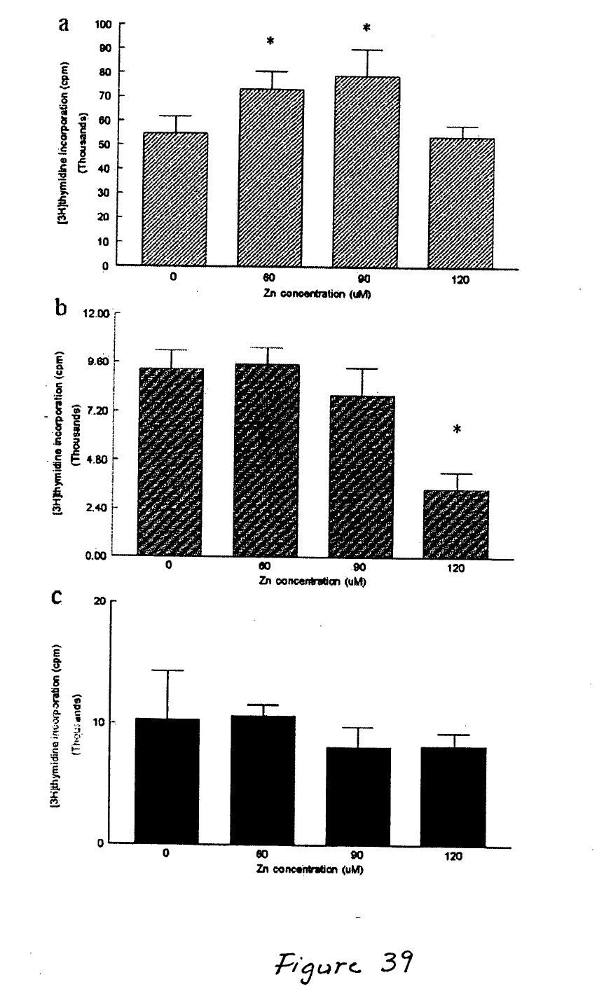

FIG. 39a: NLS-deficient p21 transfected cells (pMTCB6-ΔNLSp21; clone 9) were serum starved and incubated with the indicated concentrations of ZnSO₄ for 2 hours. Ten percent serum-containing medium was added for another 24 hours and [³H]thymidine incorporation was assessed as described in Section 2. FIG. 39b: Full-length p21 transfected cells (pMTCB6-p21 -full) were serum starved and incubated with the indicated concentrations of ZnSO₄ for 2 hours. Ten percent serum-containing medium was added for another 24 hours and [³H]thymidine incorporation was assessed as described. FIG. 39c: Empty-vector transfected cells (pMTCB6+) were serum starved and incubated with the indicated concentrations of ZnSO₄ for 2 hours. Ten percent serum-containing medium was added to all cells for another 24 hours and [³H]thymidine incorporation was assessed as described in Section 2. Each data point represents triplicate wells; these experiments are representative of at least three separate experiments. *p<0.05 compared to 0 Zn.

Serum starved cells from clone 9 of the pMTCB6-ΔNLSp21 transfectants as well as pMTCB6+ (empty vector) and pMTCB6-p21-full (full-length p21) clones were stimulated with 10% serum 2 hours after the addition of ZnSO₄ at several concentrations. Cell cycle transit was assessed by [³H]thymidine incorporation. pMTCB6-ΔNLSp21-transfected cells demonstrated a significant increase in cell cycle transit, with a dose-dependent augmentation of [³H]thymidine incorporation, when incubated with concentrations of ZnSO₄ from 0 to 90 μM (FIG. 39a). Both pMTCB6-p21-full (full-length p21; FIG. 39b) and pMTCB6+ (empty vector; FIG. 39c) clones did not show an increase in [³H]thymidine incorporation with increasing ZnSO₄ concentrations. In comport with the finding of full-length p21 overexpression being located in the nucleus (see FIG. 37) and in light of other reports of nuclear p21 being growth inhibitory (Zhou B P, Liao Y, Xia W, Spohn B, Lee M H, Hung M C. Nat Cell Biol 2001;3:245-52), there was marked cell cycle inhibition in the full-length p21 transfected cells at higher Zn concentrations (compare 120 μM to 60 and 90 μM Zn; FIG. 39b). At a ZnSO₄ concentration of 120 μM, there was no significant Zn toxicity as evidenced by no effect on [³H]thymidine incorporation in the empty vector cells (FIG. 39c).

In order to determine whether this effect was a general phenomenon of ΔNLS-p21 transfected cells and not due to selection of a particular transformed clone, experiments were next conducted to study the effect of serum addition on DNA synthesis in all three ΔNLS-p21 clones which showed greater cytosolic localization of p21 in response to Zn. All three ΔNLSp21 clones show similar effects on proliferation. All three NLS-deficient p21 transfected cells (pMTCB6-ΔNLSp21; clones 1, 7 and 9) were serum starved and incubated with the indicated concentrations of ZnSO₄ for 2 hours. Ten percent serum-containing medium was added for another 24 hours and [³H]thymidine incorporation was assessed as described. Each data point represents triplicate wells; these experiments are representative of at least three separate experiments. *p<0.05 compared to 0 Zn for each clone.

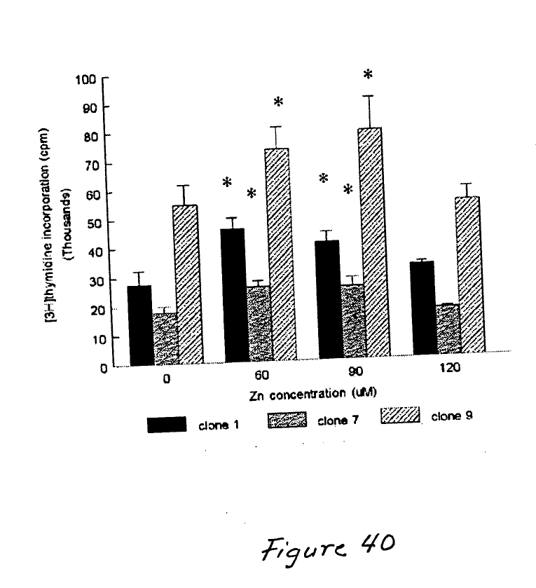

All of these clones. (clones 1, 7 and 9) demonstrated similar qualitative Zn responsiveness up to 90 μM Zn with a relative decrease in [³H]thymidine incorporation at maximal stimulation of 120 μM Zn (FIG. 40). While overexpression of full-length p21 at 120 μM Zn behaved as expected (see FIG. 39b), marked overexpression of ΔNLS-p21 also resulted in a relative growth inhibitory effect in all ΔNLS-p21 clones (FIG. 40; 120 μM as compared to 60 and 90 μM of Zn) possibly due to its entry into the nucleus when present in abundance.

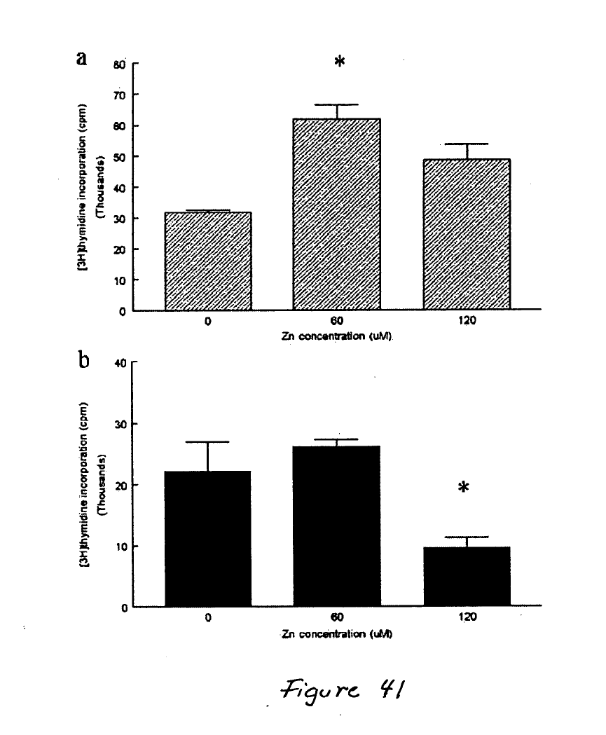

Because the VSM cells studied were exquisitely sensitive to the mitogenic effect of PDGF-BB, the effect of a stronger, combined mitogenic stimulus on the ΔNLS and full-length p21 transfected cells was examined next. Cells were serum-starved, incubated with Zn for 2 hours, and then stimulated with both 30 ng/ml PDGF and 10% serum simultaneously. ΔNLSp21 clones show increased growth with a stronger mitogenic stimulus. FIG. 41a: NLS-deficient p21 transfected cells (pMTCB6-ΔNLSp21; clone 9) were serum starved and incubated with the indicated concentration of ZnSO₄ for 2 hours. Subsequently, PDGF-BB (30 ng/ml) and 10% serum-containing medium were added to all cells for another 24 hours and [³H]thymidine incorporation was assessed. FIG. 41b: Full-length p21 transfected cells (pMTCB6-p21-full) were serum starved and incubated with the indicated concentration of ZnSO₄ for 2 hours. Subsequently, PDGF-BB (30ng/ml) and 10% serum-containing medium were added to all cells for another 24 hours and [³H]thymidine incorporation was assessed. Each data point represents triplicate wells; these experiments are representative of at least three separate experiments. *p<0.05 compared to 0 Zn.

As in the serum alone stimulated cells, there was marked augmentation of proliferation in pMTCB6-ΔNLS-p21 cells incubated with 60 μM Zn, with attenuation of maximal growth in cells incubated with 120 μM Zn (FIG. 41) as before (FIG. 39).

In summary, the above Example VII demonstrates that increased p21 levels may be utilized to promote cell cycle transit. Using a mutant construct of p21, which lacked the nuclear-localization signal (NLS), thereby increasing cytoplasmic levels of p21 in tranfected VSM cells, it was determined that cytosolic localization of p21 conferred a proliferative signal in VSM cells. As such regulation of p21 levels in VSM cells can be used to control increases in cell cycle transit.

1. A method for inhibiting cyclin-dependent kinase-mediated cell growth and proliferation, comprising inhibiting the activity and/or production of a p21^{Waf1/Cip1} protein with a p21^{Waf1/Cip1} inhibitory agent in an amount effective to inhibit cell growth and proliferation.

2. The method of claim 1, wherein said p21^{Waf1/Cip1} inhibitory agent is an antisense oligonucleotide molecule directed to the nucleotide sequence of p21^{Waf1/Cip1}.

3. The method of claim 1, wherein said p21^{Waf1/Cip1} inhibitory agent is an antibody directed against the p21^{Waf1/Cip1} protein.

4. The method of claim 3, wherein the antibody is a monoclonal antibody.

5. The method of claim 3, wherein the monoclonal antibody comprises murine antigen binding region residues and human antibody residues.

6. The method of claim 3, wherein the monoclonal antibody is a humanized antibody.

7. The method of claim 3, wherein the monoclonal antibody is a human antibody.

8. A method of inhibiting a disease associated with abnormal cell growth and proliferation, comprising inhibiting p21^{Waf1/Cip1} protein by administering a p21^{Waf1/Cip1} inhibitory agent in an amount effective to inhibit cell growth and proliferation.

9. The method of claim 8, wherein said p21^{Waf1/Cip1} inhibitory agent is an antisense oligonucleotide molecule directed to the nucleotide sequence of p21^{Waf1/Cip1}.

10. The method of claim 8, wherein said p21^{Waf1/Cip1} inhibitory agent is an antibody directed against the p21^{Waf1/Cip1} protein.

11. The method of claim 10, wherein the antibody is a monoclonal antibody.

12. The method of claim 10, wherein the monoclonal antibody comprises murine antigen binding region residues and human antibody residues.

13. The method of claim 10, wherein the monoclonal antibody is a humanized antibody.

14. The method of claim 10, wherein the monoclonal antibody is a human antibody.

15. The method of claim 8, wherein the disease is a fibrotic disease.

16. The method of claim 15, wherein the disease is selected from the group comprising of atherosclerosis, angioplasty restenosis, and renal mesangial cell proliferation.

17. The method of claim 8, wherein the disease is cancer.

18. The method of claim 8, further comprising exposing the cells to radiation.

19. The method of claim 8, further comprising administering a chemotherapeutic drug.

20. A method of inhibiting angiogenesis, comprising inhibiting p21^{Waf1/Cip1} protein by administering a p21^{Waf1/Cip1} inhibitory agent in an amount effective to inhibit angiogensis in tumors.

21. The method of claim 20, wherein said p21^{Waf1/Cip1} inhibitory agent is an antisense oligonucleotide molecule directed to the nucleotide sequence of p21^{Waf1/Cip1}.

22. The method of claim 20, wherein said p21^{Waf1/Cip1} inhibitory agent is an antibody directed against the p21^{Waf1/Cip1} protein.

23. The method of claim 22, wherein the antibody is a monoclonal antibody.

24. The method of claim 22, wherein the monoclonal antibody comprises murine antigen binding region residues and human antibody residues.

25. The method of claim 22, wherein the monoclonal antibody is a humanized antibody.

26. The method of claim 22, wherein the monoclonal antibody is a human antibody.

27. A method of inhibiting the growth of tumor cells, comprising administering to a patient an p21^{Waf1/Cip1} inhibitory agent in an amount effective to inhibit growth of the tumor cells.

28. The method of claim 27, wherein said p21^{Waf1/Cip1} inhibitory agent is an antisense oligonucleotide molecule directed to the nucleotide sequence of p21^{Waf1/Cip1}.

29. The method of claim 27, wherein said p21^{Waf1/Cip1} inhibitory agent is an antibody directed against the p21^{Waf1/Cip1} protein.

30. The method of claim 29, wherein the antibody is a monoclonal antibody.

31. The method of claim 29, wherein the monoclonal antibody comprises murine antigen binding region residues and human antibody residues.

32. The method of claim 29, wherein the monoclonal antibody is a humanized antibody.

33. The method of claim 29, wherein the monoclonal antibody is a human antibody.

34. The method of claim 27, further comprising administering a chemotherapeutic drug.

35. The method of claim 27, further comprising administering radiation therapy.

36. A pharmaceutical composition comprising a p21^{Waf1/Cip1} inhibitory agent.

37. The composition of claim 36, wherein said inhibitory agent is an antisense oligonucleotide molecule directed to the nucleotide sequence of p21^{Waf1/Cip1}.

38. The composition of claim 36, wherein said inhibitory agent is an antibody directed against the p21^{Waf1/Cip1} protein.

39. The composition of claim 38, wherein said antibody is a monoclonal antibody.

40. A method for inducing apoptosis of a cell by inhibiting cyclin-dependent kinase-mediated cell growth and proliferation by the method of claim 1.

41. A method for inhibiting production of a matrix protein by a cell by inhibiting cyclin-dependent kinase-mediated cell growth and proliferation by the method of claim 1.


SEQUENCE LISTING (8 sequences)

SEQ ID NO: 1 - DNA, 21 bp, Artificial
  antisense p21Waf1/Cip1 oligodeoxynucleotide
gacatcaccaggatcggacat

SEQ ID NO: 2 - DNA, 21 bp, Artificial
  sense p21Waf1/Cip1 oligodeoxynucleotide
atgtccgatcctggtgatgtc

SEQ ID NO: 3 - DNA, 18 bp, Artificial
  random sequence oligodeoxynucleotide
tggatccgacatgtcaga

SEQ ID NO: 4 - DNA, 21 bp, Artificial
  Human antisense p21
atccccagccggttctgacat

SEQ ID NO: 5 - DNA, 21 bp, Artificial
  Human p21 sense
tacagtcttggccgaccccta

SEQ ID NO: 6 - DNA, 19 bp, Artificial
  Human NLS p21 sense
acctcacctgctctgctgc

SEQ ID NO: 7 - DNA, 19 bp, Artificial
  Human NLS p21 antisense
gactgcaggcttcctgtgg

SEQ ID NO: 8 - protein, 4 aa, Artificial
  Caspase-3 substrate sequence
DEVD